(19) United States
(12) Patent Application Publication
(10) Pub. No.: US 2025/0058033 A1
(43) Pub. Date: Feb. 20, 2025

(54) SYSTEMS AND METHODS FOR PERITONEAL DIALYSIS WITH CONTINUOUS FLOW

(71) Applicant: Lukasz R. Kiljanek, Chesapeake Beach, MD (US)

(72) Inventor: Lukasz R. Kiljanek, Chesapeake Beach, MD (US)

(21) Appl. No.: 18/807,890

(22) Filed: Aug. 16, 2024

Related U.S. Application Data
(60) Provisional application No. 63/532,906, filed on Aug. 16, 2023.

(51) Int. Cl. A61M 1/28; A61M 1/16; A61M 1/26; A61M 1/34; G06N 20/00

(52) U.S. Cl. CPC A61M 1/284; A61M 1/1607; A61M 1/1656; A61M 1/267; A61M 1/285; A61M 1/3437; G06N 20/00; A61M 2205/18

(57) ABSTRACT

Systems and methods are disclosed for peritoneal dialysis. In an illustrative example, a peritoneal dialysis system can include an input lumen that receives a fluid from a first portion of a peritoneal cavity of a patient. The peritoneal dialysis system includes a filter that filters the fluid to produce a filtered fluid and a waste product. The peritoneal dialysis system includes a mixer that adds a predetermined amount of at least one electrolyte to the filtered fluid to produce a dialysate. The peritoneal dialysis system includes a sensor that measures one or more characteristics of the dialysate, and a controller that compares the one or more characteristics to one or more dialysate characteristic thresholds to verify that the dialysate satisfies one or more rules. The peritoneal dialysis system includes an output lumen that provides the dialysate to a second portion of the peritoneal cavity of the patient.

### CROSS-REFERENCE TO RELATED APPLICATIONS

The present invention claims the priority benefit of U.S. provisional patent application No. 63/532,906 filed Aug. 16, 2023 and titled “Peritoneal dialysis with continuous flow and other improvements,” the disclosure of which is incorporated by reference herein.

### FIELD

This disclosure is related to systems and methods for administering a particular treatment and/or prophylaxis (i.e., peritoneal dialysis) to a specific patient population (e.g., patients with certain types of renal disease(s) and/or dysfunction(s)). More specifically, this disclosure relates to systems and methods for peritoneal dialysis with continuous flow and dialysate generation using filter(s) (e.g., reverse osmosis filter(s)), mixer(s) (e.g., to add electrolyte(s)), and/or sensor(s) (e.g., to verify condition(s) of the dialysate and/or to determine relative position(s) of lumen(s)).

### BACKGROUND

In the human body, a primary role of the kidneys is to clean unwanted substances, such as toxic solutes (e.g., substances soluble in body fluids), from the body. In kidney diseases, a patient's kidney(s) can fail to perform this role. Dialysis is a type of treatment that helps a patient's body remove extra fluid and/or waste products from your blood, for instance when the patient's kidney(s) are not able to do so naturally with sufficient efficacy.

In patients with certain conditions, such as certain types of renal disease(s) and/or dysfunction(s) (e.g., end stage kidney disease (ESKD), also known as end stage renal disease (ESRD)) that require the patient to receive dialysis to prevent serious health risk, the patient's kidney(s) can reach a level of dysfunction in which the amount of waste products and/or toxins that accumulate in the patient's body (e.g., in the patient's blood) reaches the level incompatible with life and/or incompatible with reasonable health status. Such patients can require renal replacement therapy (RRT) to stay alive and remain at least relatively healthy. RRT can include dialysis and/or kidney transplantation.

Two types of dialysis include hemodialysis (HD) and peritoneal dialysis (PD). HD generally involves hooking a dialysis machine up to an artery and/or vein of the patient, for instance in the patient's arm. PD generally involves hooking a dialysis machine up to the peritoneal cavity of the patient.

As of 2020, nearly 908,000 people in the United States are living with ESKD (also known as ESRD), with 69% on dialysis and 31% receiving a kidney transplant. As of 2023, peritoneal dialysis (PD) is used by more than 50 percent of dialysis patients in Canada, but is used by less than 7 percent of dialysis patients in the United States.

### SUMMARY

The detailed description is set forth with reference to the accompanying figures. In the figures, the left-most digit(s) of a reference number identifies the figure in which the reference number first appears. The use of the same reference numbers in different figures indicates similar or identical items or features. Moreover, multiple instances of the same part are designated by a common prefix, in some cases separated from the instance number by a dash and/or parentheses. The drawings are not to scale.

Systems and techniques are described for peritoneal dialysis. In some examples, a peritoneal dialysis system includes an input lumen that is configured to, and can, receive a fluid from a first portion of a peritoneal cavity of a patient. The peritoneal dialysis system includes a filter that is configured to, and can, filter the fluid to produce a filtered fluid and a waste product. The peritoneal dialysis system includes a mixer that is configured to, and can, add a predetermined (or dynamic) amount of at least one electrolyte to the filtered fluid to produce a dialysate. The peritoneal dialysis system includes a sensor that is configured to, and can, measure one or more characteristics of the dialysate. The peritoneal dialysis system includes a controller (e.g., a processor that executes instructions stored in a memory) that is configured to, and can, compare the one or more characteristics to one or more dialysate characteristic thresholds to verify that the dialysate satisfies one or more rules. The peritoneal dialysis system includes an output lumen that is configured to, and can, provide the dialysate to a second portion of the peritoneal cavity of the patient.

According to at least one example, an apparatus is provided. The apparatus includes a first lumen that receives a fluid from a first portion of a peritoneal cavity of a patient; a filter that filters the fluid to divide the fluid into a filtered fluid and a waste product; a mixer that adds a predetermined amount of at least one electrolyte to the filtered fluid to produce a dialysate; a sensor that measures one or more characteristics of the dialysate; a processor that executes instructions stored in a memory, wherein execution of the instructions by the processor causes the processor to compare the one or more characteristics to one or more dialysate characteristic thresholds to verify that the dialysate satisfies one or more rules; and a second lumen that provides the dialysate to a second portion of the peritoneal cavity of the patient.

In another example, a method is provided. The method includes: receiving a fluid from a first portion of a peritoneal cavity of a patient through a first lumen; filtering the fluid using a filter to divide the fluid into a filtered fluid and a waste product; adding a predetermined (or dynamic) amount of at least one electrolyte to the filtered fluid to produce a dialysate; measuring one or more characteristics of the dialysate using a sensor; comparing the one or more characteristics to one or more dialysate characteristic thresholds to verify that the dialysate satisfies one or more rules; and providing the dialysate to a second portion of the peritoneal cavity of the patient through a second lumen.

In another example, a non-transitory computer-readable medium is provided that has stored thereon instructions that, when executed by one or more processors, cause the one or more processors to: receive a fluid from a first portion of a peritoneal cavity of a patient through a first lumen; filter the fluid using a filter to divide the fluid into a filtered fluid and a waste product; add a predetermined (or dynamic) amount of at least one electrolyte to the filtered fluid to produce a dialysate; measure one or more characteristics of the dialysate using a sensor; compare the one or more characteristics to one or more dialysate characteristic thresholds to verify that the dialysate satisfies one or more rules; and provide the dialysate to a second portion of the peritoneal cavity of the patient through a second lumen.

In another example, an apparatus is provided. The apparatus includes: means for receiving a fluid from a first portion of a peritoneal cavity of a patient; means for filtering the fluid to divide the fluid into a filtered fluid and a waste product; means for adding a predetermined (or dynamic) amount of at least one electrolyte to the filtered fluid to produce a dialysate; means for measuring one or more characteristics of the dialysate; means for comparing the one or more characteristics to one or more dialysate characteristic thresholds to verify that the dialysate satisfies one or more rules; and means for providing the dialysate to a second portion of the peritoneal cavity of the patient.

This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

The foregoing, together with other features and aspects, will become more apparent upon referring to the following specification, claims, and accompanying drawings.

### BRIEF DESCRIPTION OF THE DRAWINGS

The detailed description is set forth with reference to the accompanying figures. In the figures, the left-most digit(s) of a reference number identifies the figure in which the reference number first appears. The use of the same reference numbers in different figures indicates similar or identical items or features. Moreover, multiple instances of the same part are designated by a common prefix, in some cases separated from the instance number by a dash and/or parentheses. The drawings are not to scale.

The accompanying drawings are presented to aid in the description of various aspects of the disclosure and are provided solely for illustration of the aspects and not limitation thereof. So that the above-recited features of the present disclosure can be understood in detail, a more particular description, briefly summarized above, may be had by reference to aspects, some of which are illustrated in the appended drawings. It is to be noted, however, that the appended drawings illustrate only certain typical aspects of this disclosure and are therefore not to be considered limiting of its scope, for the description may admit to other equally effective aspects. The same reference numbers in different drawings may identify the same or similar elements.

FIG. 1 is a block diagram illustrating an example of a use case of a peritoneal dialysis system that includes an inflow lumen, an outflow lumen, a recycler, and a sorter, in accordance with some examples;

FIG. 2A is a block diagram illustrating an example of a use case of a peritoneal dialysis system that includes first lumen sensor(s), second lumen sensor(s), outflow pump(s) Pₒ, inflow pump(s) Pᵢ, and a sorter that includes sensor(s) S_{A}, sensor(s) S_{B}, and valves (e.g., valve V_{A}, valve V_{B}, valve V_{C}, and valve V_{D}), in accordance with some examples;

FIG. 2B is a block diagram illustrating an example of a use case of the peritoneal dialysis system of FIG. 2A in which the two lumens of the peritoneal dialysis system are arranged differently within the peritoneal cavity compared to FIG. 2A, and therefore the peritoneal dialysis system designates different lumens (compared to FIG. 2A) to be the outflow lumen and the inflow lumen, respectively, in accordance with some examples;

FIG. 3 is a block diagram illustrating an example of a use case of a peritoneal dialysis system that includes a lumen that can be used for inflow and outflow, in accordance with some examples;

FIG. 4 is a block diagram illustrating an example of a use case of a peritoneal dialysis system that includes two inflow lumens and an outflow lumen, each with corresponding lumen sensors and corresponding sensors in a sorter, in accordance with some examples;

FIG. 5 is a block diagram illustrating an example of a use case of a peritoneal dialysis system that includes two inflow lumens and two outflow lumens, in accordance with some examples;

FIG. 6 is a block diagram illustrating an example of a use case of a filter subsystem (with a recirculation loop) that can be included in a peritoneal dialysis system, in accordance with some examples;

FIG. 7 is a block diagram illustrating an example of a use case of a mixer subsystem that can be included in a peritoneal dialysis system, in accordance with some examples;

FIG. 8 is a block diagram illustrating an example of a use case of a waste evaporator subsystem that can be included in a peritoneal dialysis system, in accordance with some examples;

FIG. 9 is a table illustrating changes to sodium level before and after use of a dialysate that is sodium-based in a patient who has a high initial level of sodium (170) and with a filter that has a low rejection rate of sodium (0.90), in accordance with some examples;

FIG. 10 is a table illustrating changes to sodium level before and after use of a dialysate that is sodium-based in a patient who has a low initial level of sodium (127) and with a filter that has a low rejection rate of sodium (0.90), in accordance with some examples;

FIG. 11 is a table illustrating changes to sodium level before and after use of a dialysate that is sodium-based in a patient who has a low initial level of sodium (140) and with a filter that has a high rejection rate of sodium (0.95), in accordance with some examples;

FIG. 12 is a graph diagram illustrating changes to volume and flow over time based on use of a dialysis system, in accordance with some examples;

FIG. 13 is a conceptual diagram illustrating a scene with a patient wearing a backpack with a portable dialysis system and a patient wearing a purse with a portable dialysis system, in accordance with some examples;

FIG. 14 is a block diagram illustrating an example of a machine learning system for training and use of one or more machine learning model(s) in a peritoneal dialysis system, in accordance with some examples;

FIG. 15 is a flow diagram illustrating an example of a process for peritoneal dialysis, in accordance with some examples; and

FIG. 16 is a block diagram illustrating an example of a computing system that can implement the various techniques described herein, in accordance with some examples.

### DETAILED DESCRIPTION

The detailed description is set forth with reference to the accompanying figures. In the figures, the left-most digit(s) of a reference number identifies the figure in which the reference number first appears. The use of the same reference numbers in different figures indicates similar or identical items or features. Moreover, multiple instances of the same part are designated by a common prefix, in some cases separated from the instance number by a dash and/or parentheses. The drawings are not to scale.

As noted above, peritoneal dialysis (PD) is one of the modalities of renal replacement therapies (RRTs). PD involves hooking a dialysis machine up to the peritoneal cavity (PC) of the patient. The PC is a cavity in a patient's abdomen that includes a number of organs, including the patient's small intestines, large intestines, stomach, liver, parts of the duodenum, mesentery, omentum, gallbladder, spleen, and certain ligaments. In women, the peritoneal cavity includes certain reproductive organs as well, such as the uterus, ovaries, and fallopian tubes. In healthy patients who are not undergoing RRT, the PC generally includes a small amount of a lubricating peritoneal fluid (PF), which reduces friction between the patient's organs to prevent them from damaging each other when the organs rub against one another.

A peritoneal membrane (PM), also known as the peritoneum, is a smooth, transparent, and semi-permeable membrane that lines both the PC and the abdominal organs within the PC. The PM pads and insulates organs, helps hold organs in place, and secretes the lubricating peritoneal fluid (PF) that reduces friction between organs. PD involves using the semi-permeability of the PM to remove solutes and waste solutes (waste products) from the body. For instance, PD can involve infusing the PC with a dialysate (e.g., a clean fluid) and allowing the dialysate to stay in PC for a specified amount of time (e.g., one or more minutes, one or more hours). The dialysate can also be referred to as a dialysis fluid or as a PD fluid. As the dialysate dwells in the PC, the semipermeable characteristics of PM allows substances (e.g., the solutes, waste products, and/or excessive unneeded water) to transfer (e.g., via osmosis) from the patient (e.g., from the patient's organs) into the fluid (dialysate). Dialysate that is saturated with solutes and/or waste products from the patient's body, and/or excessive water (“ultra-filtrate”) from the patient's body, is referred to as spent dialysate (SD).

PD involves draining this spent dialysate from the patient's PC (e.g., hours after the dialysate was first introduced into the patient's PC) and once again introducing dialysate (D) that is clean and fresh into the patient's PC to replace the SD. To remove excessive fluid from the patient, also referred to as performing ultra-filtration (UF), traditional dialysates include a very high concentration of glucose, for instance with a 10× to 60× higher glucose concentration than in a healthy patient's blood. This high gradient of glucose between the dialysate and the patient's body generates an osmotic gradient, allowing for unneeded water (and/or solutes and/or waste products) to transfer from the patient's body into the dialysate in the PC as part of UF and dialysis more generally.

PD provides a number of benefits over HD. For instance, because PD does not require access to the patient's blood, risks associated with technique failure and/or infection are generally lower than with HD. Indeed, studies show that PD provides better probabilities of early survival compared to hemodialysis (HD), for instance with lower patient mortality due to bacteremia and/or sepsis. Patients can perform PD at home without needing sophisticated equipment, and can even perform PD without electricity. PD is generally performed using a PD catheter. Placement of a PD catheter is generally a simpler and safer procedure than placement of arteriovenous (AV) fistulas or AV grafts, which are generally considered necessary for chronic hemodialysis. For instance, placement of a PD catheter has a higher success rate than placement of AV fistulas and/or AV grafts, which commonly require additional procedures (e.g., surgeries) to be ready for use in HD. Once AV fistulas and/or AV grafts are placed in a patient, they generally require periodic monitoring (e.g., every 3-6 months) with invasive imaging studies (e.g., angiographies that involve contrast administration) to maintain functionality and safety. Such monitoring is not needed for a PD catheter, and PD does not require AV fistulas or AV grafts. Some studies also show that PD better preserves patient's remaining kidney function compared to HD.

The Centers for Medicare and Medicaid Services (CMS), and numerous governments around the world, support expanding PD, and are investing in developing PD further based on the benefits of PD compared to HD. In addition to the medical benefits listed above, PD is generally less expensive and less labor intensive than HD. For instance, PD does not require attendance of dialysis technician or nurse to be performed. In contrast, in-center HD requires in-center dialysis staff and resources. in 2011, the average hemodialysis cost per patient per year was 87,1045 dollars, whereas the average peritoneal dialysis cost per patient per year was 71,630 dollars.

However, traditional PD has certain drawbacks as well. For instance, traditional PD can generally only be performed on a patient for 3-5 years, after which the patient's PM becomes impermeable to solutes. This decrease in permeability of the patient's PM is believed to be caused by repeated trauma to the PM from the high concentration of glucose and glucose degradation products in the dialysate, and by peritoneal infections that occur from time to time as a result of PD. Repeated dialysate exchanges (e.g., replacing spent dialysis with fresh dialysate), and repeated connections to and disconnections from the patient's peritoneal catheter, can increase the risk of infection. For instance, with each exchange, the closes circuit and sterility of PC-to-PD-equipment loop is broken, which in turn can decrease the total duration patients can stay on PD. PD Patients also use a large volume of dialysate, for instance with patients generally using 10-15 liters (L) of dialysate per day to be able to provide adequate clearance of solutes and toxins. Dialysate is usually sold in 2-5 L bags, which can be too heavy for patients to lift, especially patients who are elderly, injured, and/or frail. A 3 L bag of dialysate can often cost $50-$100, depending on the type of dialysate and/or the supplier. Because the dialysate is heavy and is a liquid, shipping costs are generally high as well.

Systems and techniques are described for administering a particular treatment (i.e., peritoneal dialysis) to a specific patient population (e.g., patients with certain types of renal disease(s) and/or dysfunction(s)). More specifically, this disclosure relates to systems and methods for peritoneal dialysis with continuous flow and dialysate generation using filter(s) (e.g., reverse osmosis filter(s)), mixer(s) (e.g., to add electrolyte(s)), and/or sensor(s) (e.g., to verify condition(s) of the dialysate and/or to determine relative position(s) of lumen(s)). The systems and methods for peritoneal dialysis described herein can use multiple lumens, can include systems and methods to detect early signs of infection, can include systems and methods to prevent infection and trauma to peritoneal membrane, can include systems and method to regenerate spent dialysate, can include systems and methods to make new dialysate in sterile way, can include systems and methods to provide electric charge to a dialysis device without risk of infection, or a combination thereof.

For instance, in some examples, a dialysis system includes an input lumen that is configured to, and can, receive a fluid from a first portion of a peritoneal cavity of a patient. The dialysis system includes a filter that is configured to, and can, filter the fluid to produce a filtered fluid and a waste product. The dialysis system includes a mixer that is configured to, and can, add a predetermined (or dynamic) amount of at least one electrolyte to the filtered fluid to produce a dialysate. The dialysis system includes a sensor that is configured to, and can, measure one or more characteristics of the dialysate. The dialysis system includes a controller (e.g., a processor that executes instructions stored in a memory) that is configured to, and can, compare the one or more characteristics to one or more dialysate characteristic thresholds to verify that the dialysate satisfies one or more rules. The dialysis system includes an output lumen that is configured to, and can, provide the dialysate to a second portion of the peritoneal cavity of the patient.

The systems and methods disclosed herein relates to medical devices and their functions, allowing for more effective, convenient, safer and less expensive peritoneal dialysis compared to traditional peritoneal dialysis technologies. The systems and methods disclosed herein improve over traditional peritoneal dialysis technologies by introducing continuous flow dialysis with a multi-lumen catheter, systems and methods to detect early signs of infection, systems and methods to prevent infection and/or trauma to peritoneal membrane, systems and methods regenerate spent dialysate, systems and methods to make new dialysate in a sterile way, systems and methods to provide electric charge to a dialysis device without risk of infection, or a combination thereof.

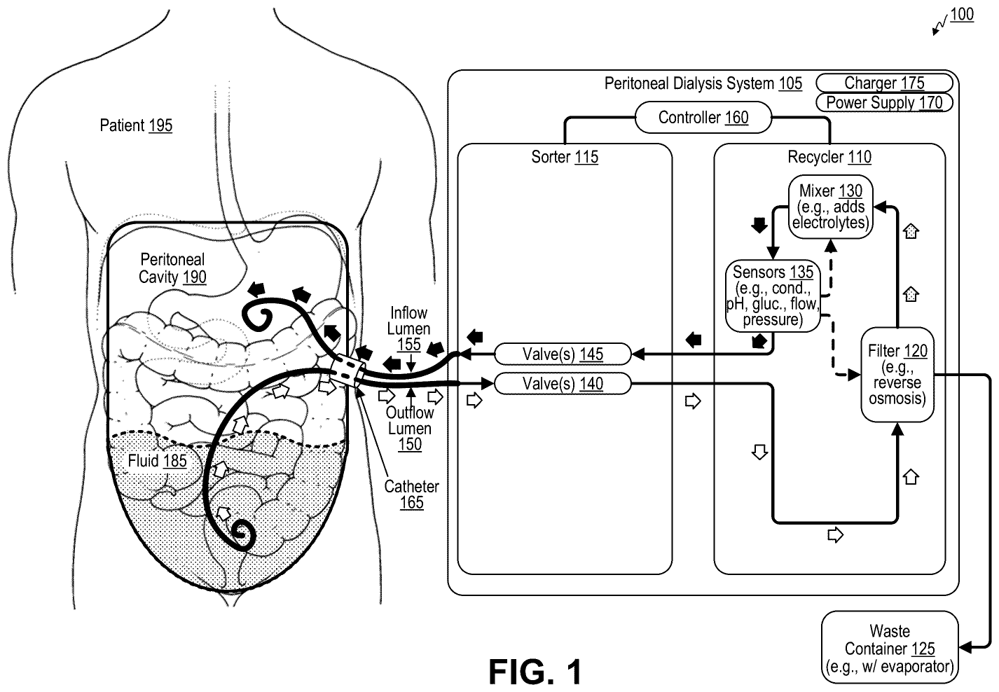

FIG. 1 is a block diagram illustrating an example of use case 100 of a peritoneal dialysis system 105 that includes an outflow lumen 150, an inflow lumen 155, a recycler 110, and a sorter 115. The peritoneal dialysis system 105 receives a fluid 185 from a peritoneal cavity 190 of a patient 195 through the outflow lumen 150. The fluid 185 can be peritoneal fluid, dialysate (e.g., spent dialysate and/or fresh dialysate), or a combination thereof. The sorter 115 includes valve(s) 140 that route the fluid 185 received through the outflow lumen 150 to an input connector of the recycler 110. The recycler 110 recycles the fluid 185 using a recycler 110 to generate dialysate (e.g., recycles spent dialysate to generate fresh, clean dialysate). The sorter 115 includes valve(s) 145 that route the dialysate generated by recycler 110 (e.g., received by the sorter 115 from an output connector of the recycler 110) to the inflow lumen 155. In some examples, the valve(s) 140 and the valve(s) 145 include at least some of the same valve(s). In some examples, the valve(s) 140 and the valve(s) 145 refer to different valves. The dialysate is pushed through the inflow lumen 155 to the peritoneal cavity 190 of the patient 195. In some examples, the peritoneal dialysis system 105 is portable, for instance fitting into a backpack, purse, or other housing that is wearable by the patient 195 (e.g., on the back of the patient 195, on an arm of the patient 195, on a leg of the patient 195, on the torso of the patient 195, on the abdomen of the patient 195, around the neck of the patient 195, or a combination thereof).

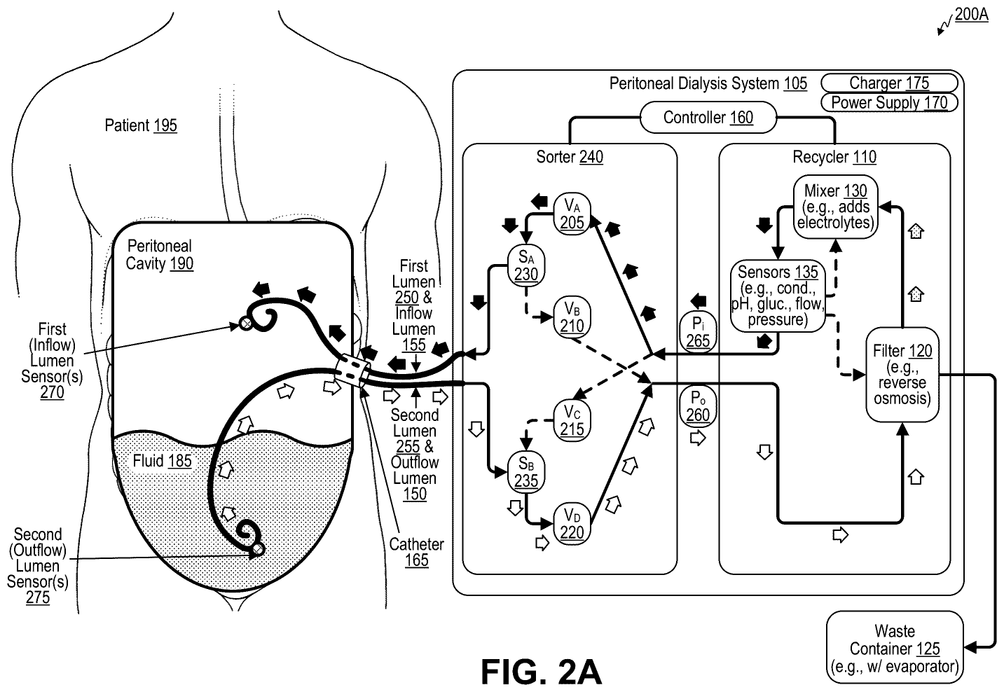

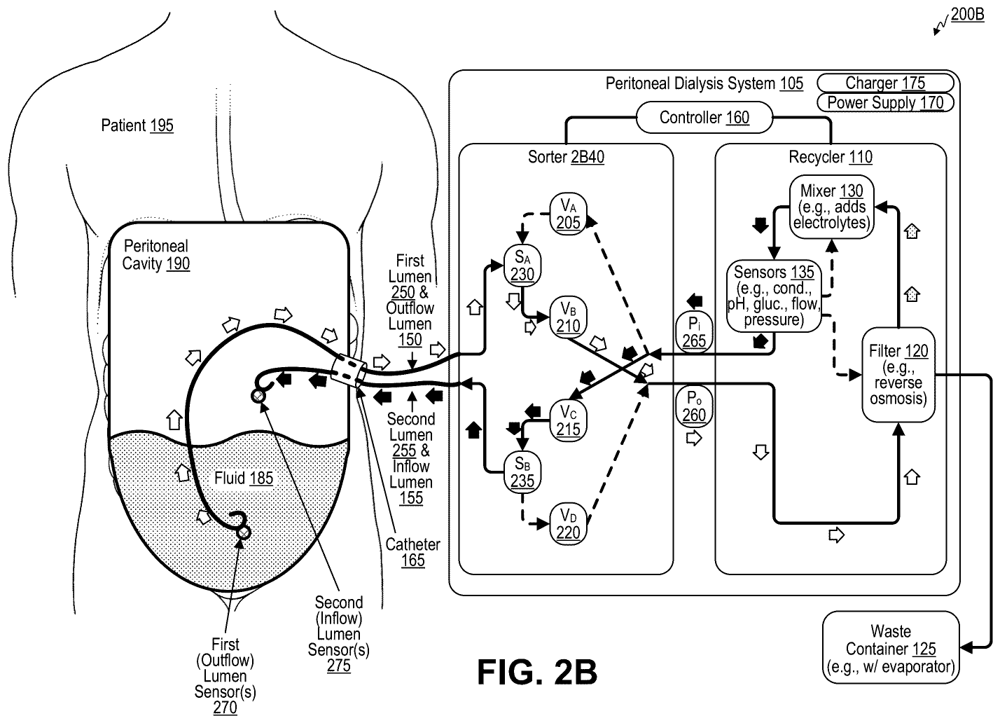

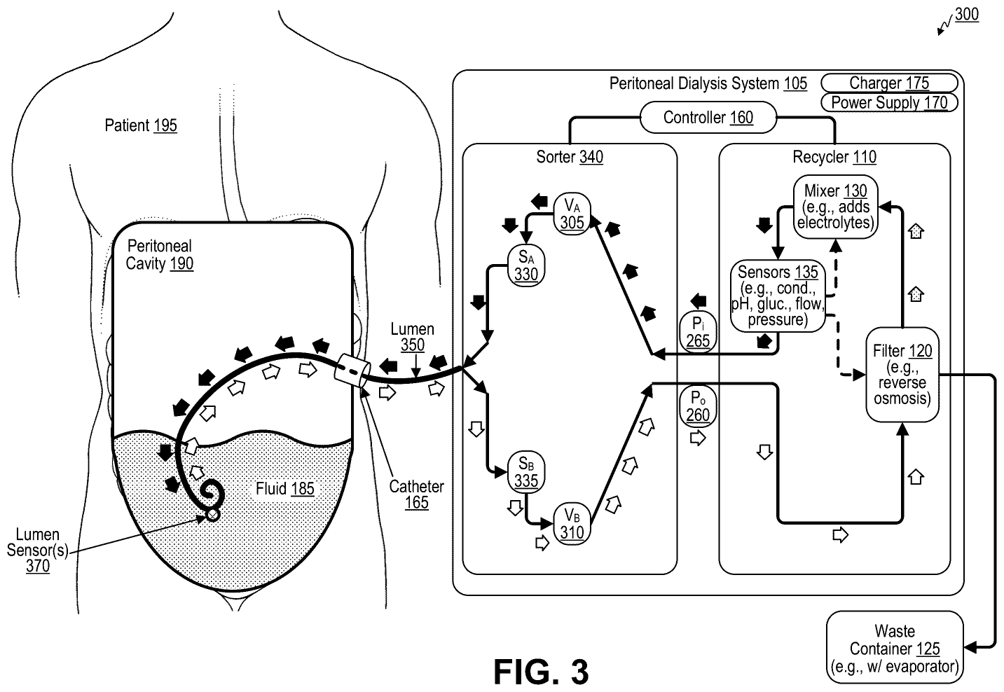

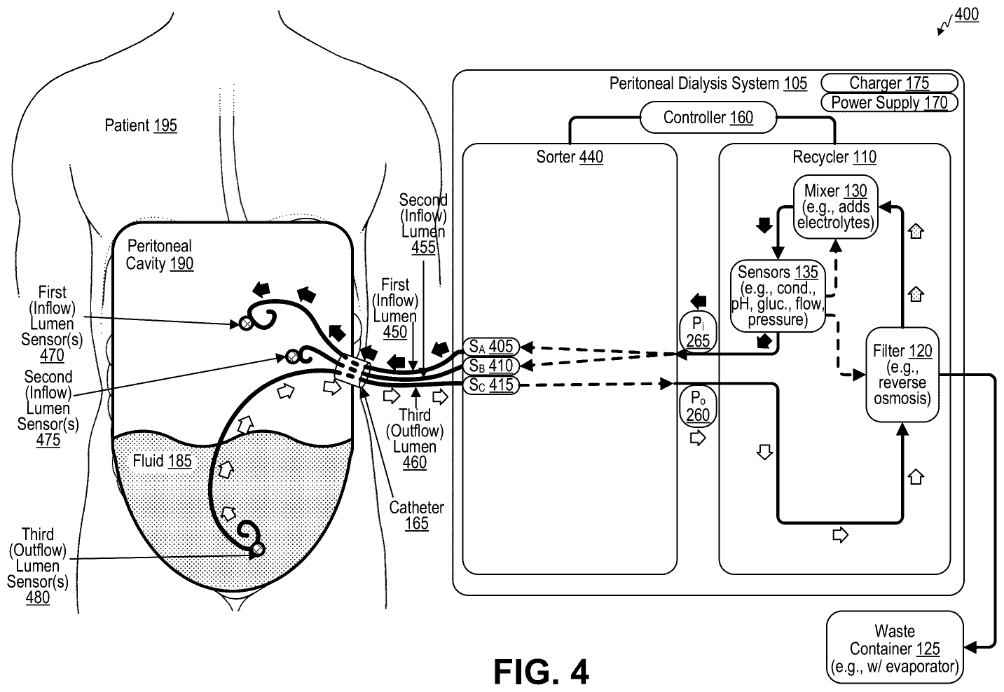

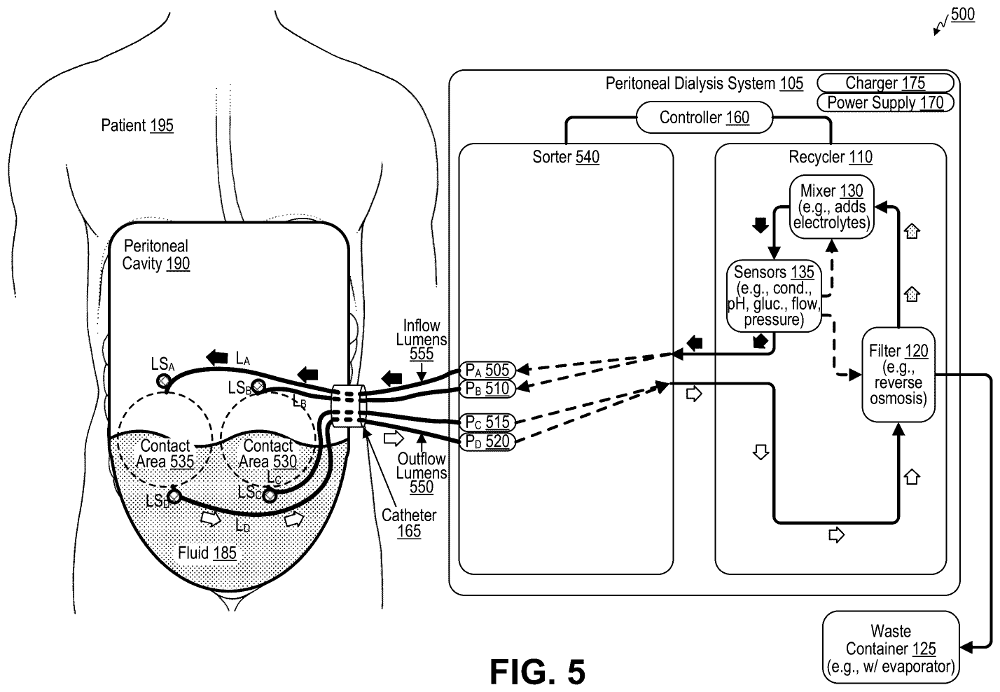

The outflow lumen 150 and the inflow lumen 155 can pass into the peritoneal cavity 190 of the patient 195 as a part of a catheter 165. The catheter 165 is shown short to save space on figure for other important elements, but usually, it is longer than illustrated and extends at least from sensors or sorter all the way to the abdominal wall of the patient. In some examples, the catheter 165 can encase, encircle, and/or house the lumens so that only a single hole (which may referred to as an exit site) need be made in the body of the patient 195 for the catheter to enter into (rather than one hole per lumen). The catheter 165 can be referred to as a dual-lumen catheter, a multi-lumen catheter, a multiple-lumen catheter, a dual-lumen peritoneal catheter, a multi-lumen peritoneal catheter, a multiple-lumen peritoneal catheter, a dual-lumen peritoneal dialysis catheter, a multi-lumen peritoneal dialysis catheter, a multiple-lumen peritoneal dialysis catheter, a dual-lumen dialysis catheter, a multi-lumen dialysis catheter, a multiple-lumen dialysis catheter, or a combination thereof. In some examples, the catheter 165 may include more than the two lumens illustrated in FIG. 1 and FIGS. 2A-2B (e.g., three lumens as in FIG. 4, four lumens as in FIG. 5, or more than four lumens) or just a single lumen (e.g., as in FIG. 3). In some examples, each lumen can function as its own independent catheter, for instance with each lumen entering the body of the patient 195 through a separate hole (or exit site).

The recycler 110 of FIG. 1 includes a filter 120 that filters out waste products (e.g., solutes and/or excess fluids) from the fluid to generate a filtered fluid. In some examples, the recycler 110 (e.g., the filter 120) routes the waste products in a waste container 125 that stores the waste products. The recycler 110 of FIG. 1 includes a mixer 130 that adds substances such as electrolytes (or in some examples, pure water) to the filtered fluid to generate the dialysate. In some examples, the recycler 110 includes sensor(s) 135 that to verify condition(s) of the dialysate as being appropriate to be input into the peritoneal cavity 190 of the patient 195. In some examples, the peritoneal dialysis system 105 includes a controller 160 that can control elements of the recycler 110 and/or of the sorter 115. For instance, the controller 160 can control operation(s) of the filter(s) 120, the mixer 130, the sensor(s) 135, the valve(s) 140, and/or the valve(s) 145. The controller 160 can be, or can include, a computing system 1600 (or one or more components thereof), or vice versa.

For instance, in some examples, the controller 160 can use sensor data from the sensor(s) 135 to verify that waste products (e.g., urea) have been sufficiently filtered out from the dialysate (e.g., by the filter(s) 120), and that a concentration of the waste products in the dialysate is less than a maximum threshold concentration. In some examples, if the controller 160 determines (based on the sensor data from the sensor(s) 135) that the concentration of the waste products in the dialysate is greater than the maximum threshold concentration, the controller 160 can instruct the recycler 110 to redirect the dialysate back to the filter(s) 120 for another round of filtering, and/or back to the mixer 130 (e.g., to add more water and/or electrolytes to water down the dialysate to reduce the concentration of the waste products). In some examples, the controller 160 can verify (based on the sensor data from the sensor(s) 135) that electrolytes (e.g., sodium, glucose) have been sufficiently added into the dialysate (e.g., by the mixer 130), and that a concentration of the electrolytes in the dialysate is greater than a minimum threshold concentration. In some examples, if the controller 160 determines (based on the sensor data from the sensor(s) 135) that the concentration of the electrolytes in the dialysate is less than the minimum threshold concentration, the controller 160 can instruct the recycler 110 to redirect the dialysate back to the mixer 130 to add more of the electrolytes to the dialysate. In this way, the recycler 110 can improve the quality (e.g., cleanliness and/or electrolyte concentration) of the dialysate until the dialysate is ready (e.g., has a sufficiently low level of waste products and/or a sufficiently high level of electrolytes) to be provided to the peritoneal cavity 190 of the patient 195.

In some examples, the sensor(s) 135 can include one or more conductivity sensors that measure conductivity of the dialysate, potential of hydrogen (pH) sensors that measure the pH of the dialysate, chemical concentration sensors and/or particulate sensors that measure concentration of certain chemicals and/or particulates in the dialysate, flow sensors that measure a flow of the dialysate, pressure sensors (e.g., hydrostatic pressure sensors) that measure a pressure (e.g., hydrostatic pressure) in or applied to the dialysate, temperature sensors (e.g., thermometers, thermistors, temperature transducers) that measure a temperature of the dialysate, or a combination thereof. In some examples, the chemical concentration sensors and/or particulate sensors can measure concentration, in the dialysate, of sodium (Na⁺), potassium (K⁺), chloride (Cl⁻), bicarbonate, calcium (Ca²⁺), magnesium (Mg²⁺), phosphate, other electrolytes, glucose, dextrose, lactate, acetate, other non-electrolytes, salts (e.g., NaCl, KCl), other solutes mentioned herein, other substances mentioned herein, or a combination thereof.

The peritoneal dialysis system 105 can also include a power supply 170 and/or a charger 175. In some examples, the power supply 170 includes one or more batteries, capacitors, fuel cells, solar panels, fuel containers, generators, or a combination thereof. In some examples, the power supply 170 supplies power to the recycler 110, the sorter 115, the filter(s) 120, the pump(s) and/or sensor(s) and/or evaporator(s) associated with the waste container 125, the mixer 130, the sensor(s) 135, the valve(s) 140, the valve(s) 145, pump(s) and/or sensor(s) associated with the outflow lumen 150, pump(s) and/or sensor(s) associated with the inflow lumen 155, the controller 160, other elements of the peritoneal dialysis system 105, or a combination thereof. In some examples, the peritoneal dialysis system 105 may include a charger 175 that may charge the power supply 170. In some examples, the charger 175 may be an inductive charger.

The term outflow lumen 150 as used herein refers to a lumen used to extract fluid (e.g., fluid 185) from the peritoneal cavity 190 of the patient 195. In some examples, the outflow lumen 150 may be referred to as the output lumen or the efflux lumen. The term inflow lumen 155 as used herein generally refers to elements that provide fluid (e.g., dialysate) into the peritoneal cavity 190 of the patient 195. In some examples, the inflow lumen 155 may be referred to as the input lumen or the influx lumen. In some examples, the aforementioned usage of the terms outflow, output, and efflux, and the terms inflow, input, or influx, can be reversed—for instance to refer to inputs to the peritoneal dialysis system 105 and outputs from the peritoneal dialysis system 105 instead of inputs to the peritoneal cavity 190 and outputs from the peritoneal cavity 190.

Several illustrative examples are provided below to indicate how the peritoneal dialysis system 105 (and/or variants thereof discussed herein) can be used. These illustrative examples can be combined with one another.

In a first illustrative example, the peritoneal dialysis system 105 is configured to, and can, provide continuous or semi-continuous peritoneal dialysis via a multiple-lumen peritoneal dialysis catheter (e.g., the catheter 165 with the outflow lumen 150 and the inflow lumen 155).

In a second illustrative example, the peritoneal dialysis system 105 is configured to, and can, operate as a portable dialysate maker by using the recycler 110 to make (e.g., create, generate, produce) dialysate. The recycler 110 can use filtration of the fluid 185 to extract clean water (or water that has less than a threshold amount of contaminants, such as waste products and/or solutes) from the fluid 185. The peritoneal dialysis system 105, working as a portable dialysate maker, can also use the mixer 130 to add solutes that are desirable to the filtered fluid to generate the dialysate. The solutes that the mixer 130 can add can include sodium (Na⁺), potassium (K⁺), chloride (Cl⁻), bicarbonate, calcium (Ca²⁺), magnesium (Mg²⁺), phosphate, other electrolytes, glucose, dextrose, lactate, acetate, other non-electrolytes, salts (e.g., NaCl, KCl), other solutes mentioned herein, other substances mentioned herein, or a combination thereof. In some examples, these solutes may be, or may include, ions and/or anions, such as sodium ions (Na⁺), chloride anions (Cl⁻), calcium ions (Ca²⁺), magnesium ions (Mg²⁺), other ions, other anions, or a combination thereof. In some examples, these solutes may be, or may include, molecules, such as bicarbonate, phosphate, glucose, dextrose, lactate, acetate, salts, water, other molecules, or a combination thereof. In some examples, the mixer 130 can also add fresh water from a fresh water tank or water bottle, for instance to help water down the filtered fluid further before adding the solutes (e.g., if the filtered fluid still retains more than a maximum threshold amount of an undesirable solute such as urea or another waste product). In some examples, the filter(s) 120 provide a fairly reliable rejection rate for certain solutes, and the mixer 130 can add a predetermined amount of a given substance (e.g., solute, water) to the filtered fluid without having to first measure the level(s) of the solute in the filtered fluid.

In some examples, the controller 160 can dynamically determine, based on sensor data from the sensor(s) 135 and/or other sensor(s) of the peritoneal dialysis system 105 (e.g., and/or other context data 1410) an amount of the given substance (e.g., solute, water) for the mixer 130 to add to the filtered fluid to generate the dialysate. For instance, in some examples, the controller 160 (e.g., and/or the machine learning system 1400) can periodically (e.g., a specific number of times per day, per week, per month, per year, or the like) adjust (e.g., based on sensor data and/or other context data 1410) the amount(s) of the various substance(s) that the mixer 130 adds to the filtered fluid to generate the dialysate. For instance, over time, the filter(s) 120 can become less effective, in which case the controller 160 can gradually change the amount(s) and/or composition(s) of the substance(s) that the mixer 130 adds to the filtered fluid to generate the dialysate. Similarly, the controller 160 can gradually change the amount(s) and/or composition(s) of the substance(s) that the mixer 130 adds to the filtered fluid to generate the dialysate based on changes to the patient's weight, changes to the patient's fitness activity, whether the patient is healthy or sick (e.g., experiencing swelling or other symptoms), and/or changes to any of the context data 1410 discussed herein. In some examples, the controller 160 can gradually change the amount(s) and/or composition(s) of the substance(s) that the mixer 130 adds to the filtered fluid to generate the dialysate to increase ultrafiltration, for instance by adding sodium and/or glucose, and/or by adding water to water down substance(s) that aren't getting filtered out effectively (e.g., to reduce the concentration of those substances in the dialysate).

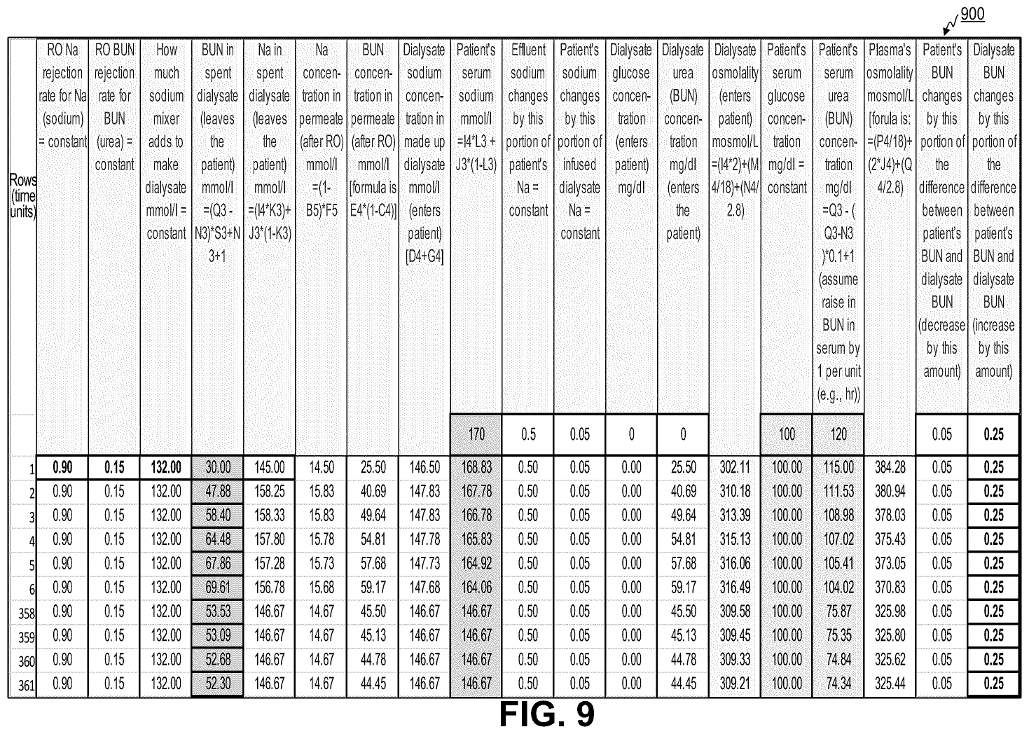

For instance, in some examples, a reverse osmosis filter (of the filter(s) 120) can reliably filter out sodium at a rejection rate of 90% to 95%. As a result, the mixer 130 can add a predetermined amount of sodium (e.g., at a concentration of 132 millimoles per liter (mmol/L)), and the resulting dialysate can reliably end up with a sodium concentration of around 147 to 149 mmol/L. This range of sodium concentration works well for patients with high, low, or normal initial sodium levels, as shown in the tables of FIGS. 9-11.

In the second illustrative example, the peritoneal dialysis system 105 is configured to, and can, operate in a closed circuit, continually recycling spent dialysate retrieved from the peritoneal cavity 190 of the patient 195 to generate fresh, clean dialysate to provide back into the peritoneal cavity 190 of the patient 195. In some examples, the peritoneal dialysis system 105 does not add glucose using the mixer 130, instead adding sodium (and/or other solutes such as those listed above) and relying on the osmotic gradient formed by the concentration of sodium (and/or the other solutes) in the dialysate vs. in the body (e.g., organs) of the patient 195. Use of sodium instead of glucose can improve outcomes for the patient 195, and/or can reduce side effects. For instance, dialysates that rely on high concentrations of glucose to form the osmotic gradient necessarily include a far higher concentration of glucose than would normally be found in the body, or in any food or beverage. Through the osmosis process that extracts waste products from the body of the patient 195 during dialysis, some of this glucose can also find its way into the body of the patient 195 through the peritoneal membrane (PM). This influx of glucose into the body of the patient 195 can cause, or contribute to, obesity, diabetes, metabolic diseases and/or dysfunctions, endocrine diseases and/or dysfunctions, gastrointestinal diseases and/or dysfunctions, or a combination thereof. Use of sodium in place of glucose in dialysate can reduce the causation or contribution to such diseases and/or dysfunctions, compared to use of glucose in dialysate.

An influx of glucose through the PM can also cause repeated trauma to the PM, reducing the permeability of the PM over time, and eventually (e.g., after 3-5 years) reducing the effectiveness of peritoneal dialysis as a result of the reduced permeability of the PM. Because sodium ions are smaller in size than glucose molecules, influx of sodium ions through the PM may cause less trauma to the PM than influx glucose through the PM, allowing the PM to preserve permeability for longer and extending (improving) the amount of time that peritoneal dialysis is effective for the patient 195. Operation in closed circuit and lack of glucose usage can also decrease the probability of infection to the patient, meaning that the peritoneal dialysis system 105 provides improved safety to the patient 195. Use of sodium rather than glucose to form the osmotic gradient for the dialysis also means that the filter(s) 120 of the peritoneal dialysis system 105, in some examples, do not need to include a desalinization system, which can be extremely heavy, large, and power-hungry. Use of sodium rather than glucose to form the osmotic gradient for the dialysis also means that the filter(s) 120 of the peritoneal dialysis system 105, in some examples, do not need to include carbon filters. Thus, use of sodium rather than glucose to form the osmotic gradient for the dialysis helps improve the peritoneal dialysis system 105 by allowing the peritoneal dialysis system 105 to be more portable, and by minimizing the amount of components (e.g. filters) that need to be replaced over time. In some examples, for instance, the peritoneal dialysis system 105 can operate continuously for one or more months, without needing anything to be replaced (other than charging of disposal of waste from the waste container 125). This continuous operation in a closed circuit can reduce the risk of infection, as risk of infection is highest when new elements are introduced to the body, thus improving safety of the peritoneal dialysis system 105 compared to traditional dialysis systems.

FIG. 2A is a block diagram illustrating an example of a use case 200A of a peritoneal dialysis system 105 that includes first lumen sensor(s) 270, second lumen sensor(s) 275, outflow pump(s) Pₒ 260, inflow pump(s) Pᵢ 265, and a sorter 240 that includes sensor(s) S_{A} 230, sensor(s) S_{B} 235, and valves (e.g., valve V_{A} 205, valve V_{B} 210, valve V_{C} 215, and valve V_{D} 220). The sorter 240 can be an example of the sorter 115, or vice versa. In some examples, the valves may be directional valves. Within FIG. 2A, the second lumen sensor(s) 275 can be referred to as outflow lumen sensor(s), referring to sensor(s) coupled to the outflow lumen 150, for instance at or near a tip of the outflow lumen 150 that is within the peritoneal cavity 190. Within FIG. 2A, first lumen sensor(s) 270 can be referred to as inflow lumen sensor(s), referring to sensor(s) coupled to the inflow lumen 155, for instance at or near a tip of the inflow lumen 155 that is within the peritoneal cavity 190.

In some examples, the catheter 165 of FIGS. 2A-2B can include two lumens, and the peritoneal dialysis system 105 (e.g., the controller 160) can dynamically determine which of the two lumens to designate to be the outflow lumen 150 and which of the two lumens to designate to be the inflow lumen 155 based on sensor data captured by the lumen sensors (e.g., the sensor(s) S_{A} 230, the sensor(s) S_{B} 235, the first lumen sensor(s) 270, and/or the second lumen sensor(s) 275). In some examples, sensor data captured by the lumen sensors can be used by the controller 160 to determine the relative positions of the two lumens of the peritoneal dialysis system 105 within the peritoneal cavity 190 of the patient 195. The controller 160 can detect, using variety of methods, including using hydrostatic tip pressure sensors and can use the relative positions of the two lumens within the peritoneal cavity 190 to designate the second lumen 255 (of the two lumens) to be the outflow lumen 150 and the first lumen 250 (of the two lumens) to the inflow lumen 155. For instance, if the controller 160 identifies, based on the sensor data from the lumen sensors, that the tip of the second lumen 255 is lower down in the peritoneal cavity 190 (following the direction of gravity) than the tip of the first lumen 250, then the controller 160 can designate the second lumen 255 (whose tip is lower down) to be the outflow lumen 150 and that the first lumen 250 should be designated as the inflow lumen 155. The lumen with the tip that is lower down in the peritoneal cavity 190 (e.g., the second lumen 255 in FIG. 2A, the first lumen 250 in FIG. 2A) can be referred to as the dependent lumen. The lumen that is dependent, or lower down, can be referred to as being positioned further within the peritoneal cavity 190 along the direction of gravity compared to the other lumen. The ability to determine which lumen is the most dependent allows dynamic adjustment of the system to patient's changing position (e.g., standing up, laying down to sleep, on left side, right side, back or abdomen). The peritoneal dialysis system 105 maintain the lumens whose tips are lowest down to be outflow lumen or lumens, and lumen or lumens with tips higher than the lowest down tip would or could remain inflow lumens.

For instance, in some examples, the lumen sensors include pressure sensor(s). If the tip of the second lumen 255 is in the fluid 185 and the tip of the first lumen 250 is out of the fluid 185 (as illustrated in FIGS. 2A-2B), the lumen sensor at the tip of the second lumen 255 will measure a higher hydrostatic pressure (caused by the lumen sensor at the tip of the second lumen 255 being immersed in the fluid 185) than the lumen sensor at the tip of the first lumen 250. This higher pressure at the tip of the second lumen 255 indicates to the controller 160 that the second lumen 255 should be designated as the outflow lumen 150 and that the first lumen 250 should be designated as the inflow lumen 155.

If the tips of both lumens are in the fluid 185, but the tip of the second lumen 255 is deeper in the fluid 185 (e.g., further into the fluid 185 in the direction of gravity) than the tip of the first lumen 250, then the lumen sensor(s) of the second lumen 255 (e.g., sensor(s) S_{B} 235 and/or the second lumen sensor(s) 275) will measure a higher hydrostatic pressure (e.g., caused by the tip of the second lumen 255, and/or the second lumen sensor 275, being immersed deeper in the fluid 185) than the first lumen sensor 270 at the tip of the first lumen 250, based also on the lumen sensor(s) of the first lumen 250 (e.g., sensor(s) S_{A} 230 and/or the first lumen sensor(s) 270). This higher hydrostatic pressure at the tip of the second lumen 255 indicates to the controller 160 that the second lumen 255 should be designated as the outflow lumen 150 (and the second lumen sensor(s) 275 at or near the tip of the second lumen 255 should be designated as the outflow lumen sensor(s)) and that the first lumen 250 should be designated as the inflow lumen 155.

In some examples, the first lumen sensor(s) 270, the second lumen sensor(s) 275, the sensor(s) S_{A} 230, and/or the sensor(s) S_{B} 235 can each include one or more conductivity sensors that measure conductivity of the fluid 185, pH sensors that measure the pH of the fluid 185, chemical concentration sensors and/or particulate sensors that measure concentration of certain chemicals and/or particulates in the fluid 185, flow sensors that measure a flow of the fluid 185, pressure sensors (e.g., hydrostatic pressure sensors) that measure a pressure (e.g., hydrostatic pressure) in or applied to the fluid 185, temperature sensors (e.g., thermometers, thermistors, temperature transducers) that measure a temperature of the fluid 185, positioning receivers such as global navigation satellite system (GNSS) receivers (e.g., global positioning system (GPS) receivers), solenoid induction triangulation sensors, accelerometers, gyrometers, gyroscopes, inertial measurement units (IMUs), or a combination thereof. The latter mentioned positioning receivers can be used in determining which lumen tip is or tips are the most dependent and which lumen shall be used for inflow and which for outflow. In some examples, the chemical concentration sensors and/or particulate sensors can measure concentration, in the dialysate, of sodium (Na⁺), potassium (K⁺), chloride (Cl⁻), bicarbonate, calcium (Ca²⁺), magnesium (Mg²⁺), phosphate, other electrolytes, glucose, dextrose, lactate, acetate, other non-electrolytes, salts (e.g., NaCl, KCl), other solutes mentioned herein, other substances mentioned herein, or a combination thereof.

In some examples, which of the two lumens of the peritoneal dialysis system 105 is designated (e.g., by the controller 160) as the outflow lumen 150 and which of the two lumens of the peritoneal dialysis system 105 is designated (e.g., by the controller 160) as the inflow lumen 155 can be different than illustrated in FIG. 2A, for instance as illustrated in FIG. 2B. In some examples, the designations of the lumens can change over time based on the lumens changing relative positions within the peritoneal cavity 190. For instance, in some examples, the lumens are at least partially flexible, and the tips of the lumens can move within the peritoneal cavity 190, so which lumen is dependent (lower down or further into the peritoneal cavity 190 in relation to the direction of gravity) can change over time even if the patient 195 does not change position or orientation. In some examples, the lumens can be in somewhat fixed positions relative to the peritoneal cavity 190, for instance being stuck to certain positions or areas (e.g., organ(s) and/or portions of the PM) within or at a periphery of the peritoneal cavity 190, for instance with an adhesive and/or fastener. Even in examples where the tips of the lumens have positions that are fixed relative to the peritoneal cavity 190, however, which lumen is dependent (lower down or further into the peritoneal cavity 190 in relation to the direction of gravity) can still change over time, for instance if the orientation of the patient 195 changes in relation to the direction of gravity. For instance, if the peritoneal dialysis system 105 has a first lumen and a second lumen, the first lumen may be dependent (compared to the second lumen) while the patient 195 is standing up or sitting down, but the second lumen may be dependent (compared to the first lumen) while the patient 195 is lying down on their side.

FIG. 2B is a block diagram illustrating an example of a use case 200B of the peritoneal dialysis system of FIG. 2A in which the two lumens of the peritoneal dialysis system 105 are arranged differently within the peritoneal cavity 190 compared to FIG. 2A, and therefore the peritoneal dialysis system 105 can automatically designate different lumens (compared to FIG. 2A) to be the outflow lumen 150 and the inflow lumen 155, respectively.

For instance, the peritoneal dialysis system 105 of FIGS. 2A-2B includes a first lumen 250 and a second lumen 255. The first lumen 250 is coupled (within the sorter 240) to the sensor(s) S_{A} 230, the valve V_{A} 205, and the valve V_{B} 210, while the second lumen 255 is coupled (within the sorter 240) to the sensor(s) S_{B} 235, the valve V_{C} 215, and the valve V_{D} 220. In FIG. 2A, the second lumen 255 is the dependent lumen because the tip of the second lumen 255 is lower in the peritoneal cavity 190 than the tip of the first lumen 250. Thus, in FIG. 2A, the second lumen 255 is designated (e.g., by the controller 160) as the outflow lumen 150, while the first lumen 250 is designated (e.g., by the controller 160) as the inflow lumen 155. In contrast, in FIG. 2B, the first lumen 250 is the dependent lumen because the tip of the first lumen 250 is lower in the peritoneal cavity 190 than the tip of the second lumen 255. Thus, in FIG. 2B, the first lumen 250 is designated (e.g., by the controller 160) as the outflow lumen 150, while the second lumen 255 is designated (e.g., by the controller 160) as the inflow lumen 155.

In situations where which lumen (between the first lumen 250 and the second lumen 255) is dependent (lower down or further into the peritoneal cavity 190 in relation to the direction of gravity) changes over time, the controller, automatically, or triggered by the input of the operator, 160 can change which lumen (between the first lumen 250 and the second lumen 255) is designated as the outflow lumen 150 and the inflow lumen 155, respectively, and can change the routing of the fluid 185 and/or the dialysate over time by modifying which of the valves (e.g., valve V_{A} 205, valve V_{B} 210, valve V_{C} 215, and valve V_{D} 220) is open or closed. For instance, in FIG. 2A, the second lumen 255 is dependent (e.g., as the controller 160 determines based on a comparison between first sensor data from the first lumen sensor(s) 270 and/or sensor(s) S_{A} 230 coupled to the first lumen 250 and second sensor data from the second lumen sensor(s) 275 and/or the sensor(s) S_{B} 235 coupled to the second lumen 255) and thus, the controller 160 designates the first lumen 250 to be the inflow lumen 155 and the second lumen 255 to be the outflow lumen 150. In FIG. 2A, the controller 160 opens valve V_{A} 205 and valve V_{D} 220, but closes valve V_{B} 210 and valve V_{C} 215. Because the valve V_{D} 220 is open in FIG. 2A, the fluid 185 retrieved from the peritoneal cavity 190 (through the second lumen 255 that is designated as the outflow lumen 150) passes through the valve V_{D} 220 toward the recycler 110. Because the valve V_{A} 205 is open in FIG. 2A, the dialysate generated by the recycler 110 passes through the valve V_{A} 205 on its way to being provided to the peritoneal cavity 190 (through the first lumen 250 that is designated as the inflow lumen 155). Note that, in the sorter 240 as illustrated in FIG. 2A, fluid channels that actively transfer fluid (e.g., fluid 185 and/or dialysate) are illustrated with solid lines (e.g., the channels around and including valve V_{A} 205 and valve V_{D} 220), while fluid channels that do not actively transfer fluid (e.g., fluid 185 and/or dialysate) are illustrated with dashed lines (e.g., the channels around and including valve V_{B} 210 and valve V_{C} 215).

On the other hand, in FIG. 2B, the first lumen 250 is dependent (e.g., as the controller 160 determines based on a comparison between first sensor data from the first lumen sensor(s) 270 and/or sensor(s) S_{A} 230 coupled to the first lumen 250 and second sensor data from the second lumen sensor(s) 275 and/or the sensor(s) S_{B} 235 coupled to the second lumen 255) and thus, the controller 160 designates the first lumen 250 to be the outflow lumen 150 and the second lumen 255 to be the inflow lumen 155. In FIG. 2B, the controller 160 opens valve V_{B} 210 and valve V_{C} 215, but closes valve V_{A} 205 and valve V_{D} 220. Because the valve V_{B} 210 is open in FIG. 2B, the fluid 185 retrieved from the peritoneal cavity 190 (through the first lumen 250 that is designated as the outflow lumen 150) passes through the valve V_{B} 210 toward the recycler 110. Because the valve V_{C} 215 is open in FIG. 2B, the dialysate generated by the recycler 110 passes through the valve V_{C} 215 on its way to being provided to the peritoneal cavity 190 (through the second lumen 255 that is designated as the inflow lumen 155). Note that, in the sorter 240 as illustrated in FIG. 2B, fluid channels that actively transfer fluid (e.g., fluid 185 and/or dialysate) are illustrated with solid lines (e.g., the channels around and including valve V_{B} 210 and valve V_{C} 215), while fluid channels that do not actively transfer fluid (e.g., fluid 185 and/or dialysate) are illustrated with dashed lines (e.g., the channels around and including valve V_{A} 205 and valve V_{D}) 220).

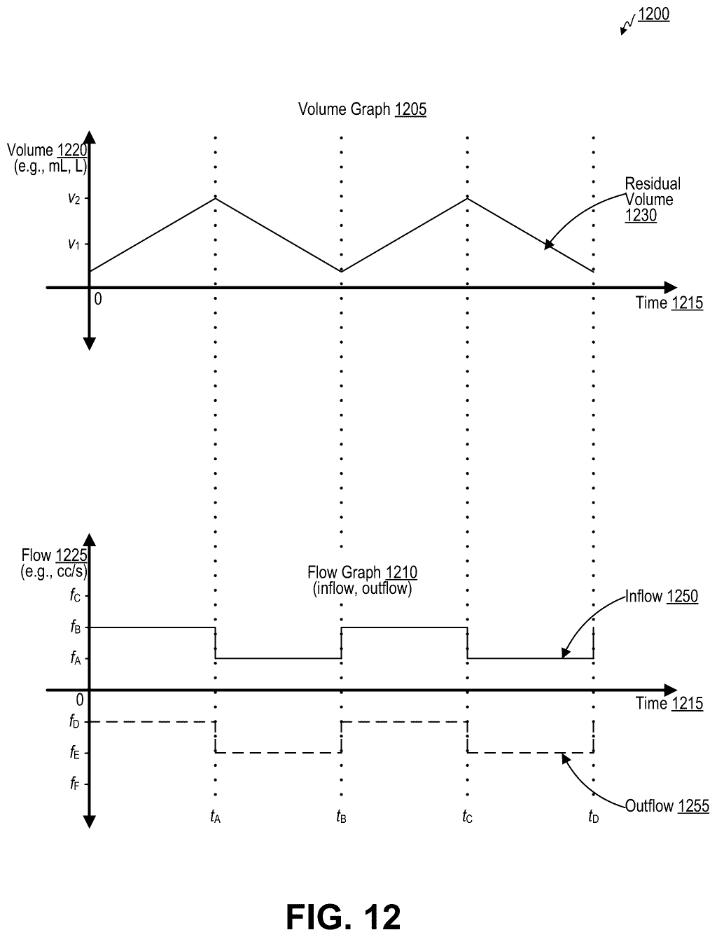

In some examples, if the controller 160 cannot determine which of the two lumens of the peritoneal dialysis system 105 (between the first lumen 250 and the second lumen 255) is dependent (and therefore which lumen to designate as the outflow lumen 150 and which lumen to designate as the inflow lumen 155), the controller 160 can switch modes of the peritoneal dialysis system 105 from a divided inflow/outflow mode to a multifunctional lumen mode. In the divided inflow/outflow mode, one lumen is designated to be an outflow lumen 150 (e.g., the second lumen 255 in FIG. 2A, the first lumen 250 in FIG. 2B) through which the peritoneal dialysis system 105 retrieves the fluid 185 from the peritoneal cavity 190, and another lumen is designated to be an inflow lumen 155 (e.g., the first lumen 250 in FIG. 2A, the second lumen 255 in FIG. 2B) through which the peritoneal dialysis system 105 provides dialysate to the peritoneal cavity 190. In the multifunctional lumen mode, one or both lumens can be used for both outflow (retrieval of the fluid 185 from the peritoneal cavity 190 to the peritoneal dialysis system 105) and inflow (provision of the dialysate from the peritoneal dialysis system 105 to the peritoneal cavity 190) at different times. For instance, in some cases, the two lumens can arrange themselves to be at similar heights relative to gravity (e.g., within a threshold height of one another), so the controller 160 cannot determine (e.g., with confidence above a confidence threshold) that one of the lumens is dependent (lower down in the direction of gravity) with respect to the other lumen. In the multifunctional lumen mode, the controller 160 can activate the outflow pump Pₒ 260 to retrieve the fluid 185 from the peritoneal cavity 190 (through one or both of the first lumen 250 and/or the second lumen 255) during a first time period, and can activate the inflow pump Pᵢ 265 to provide the dialysate from the recycler 110 to the peritoneal cavity 190 (through one or both of the first lumen 250 and/or the second lumen 255) during a second time period. In some examples, where a peritoneal dialysis system 105 has only one lumen (e.g., as in FIG. 3), the peritoneal dialysis system 105 can permanently operate in multifunctional lumen mode. In some examples, when one or more lumens clog, or become unusable for another reason, controller can provide dialysis using one lumen of the catheter only, repeating sequence of operations including: infusing dialysate set volume bolus during set time, dwelling dialysate bolus in PC for set time, and draining it whole or predetermined part of it during set time. To detect these circumstances, within its program, controller can compare sensor pressure, to pass threshold of high and very higher pressures, with normal pump operation. One lumen operation can also be triggered by the operator, or by the patient on demand. Examples of pressure and flow controls are illustrated in FIG. 12, for example.

In some examples, if the controller 160 cannot determine which of the two lumens of the peritoneal dialysis system 105 (between the first lumen 250 and the second lumen 255) is dependent (and therefore which lumen to designate as the outflow lumen 150 and which lumen to designate as the inflow lumen 155), the controller 160 can maintain the peritoneal dialysis system 105 in the divided inflow/outflow mode, and can designate the lumen that appears to be dependent based on the sensor data (e.g., even if the controller 160 has low confidence in its determination that this lumen is dependent) to be the outflow lumen 150, and the other lumen to be the inflow lumen 155. In some examples, if the controller 160 cannot determine which of the two lumens of the peritoneal dialysis system 105 (between the first lumen 250 and the second lumen 255) is dependent (and therefore which lumen to designate as the outflow lumen 150 and which lumen to designate as the inflow lumen 155), the controller 160 can maintain the peritoneal dialysis system 105 in the divided inflow/outflow mode, and can keep whichever lumen was previously designated as the outflow lumen 150 to remain as the outflow lumen 150, and can keep whichever lumen was previously designated as the inflow lumen 155 to remain as the inflow lumen 155. These designations can keep the peritoneal dialysis system 105 working even if the sensor(s) erroneously measure data that appears to be an outlier, for instance. In some examples, if the controller 160 cannot determine which of the two lumens of the peritoneal dialysis system 105 (between the first lumen 250 and the second lumen 255) is dependent (and therefore which lumen to designate as the outflow lumen 150 and which lumen to designate as the inflow lumen 155), the controller 160 can maintain the peritoneal dialysis system 105 in the divided inflow/outflow mode, and can randomly (e.g., using a random number generator) designate one lumen to be the outflow lumen 150, and the other lumen to be the inflow lumen 155.

The controller 160 can operate the outflow pump(s) Pₒ 260 to pull and/or push the fluid 185 from the peritoneal cavity 190, through the outflow lumen 150, through the sorter 240, and into the recycler 110 to be recycled (e.g., processed using the filter(s) 120, the mixer 130, and/or the sensor(s) 135) to generate dialysate. The controller 160 can operate the inflow pump(s) Pᵢ 265 to push and/or pull the dialysate from the recycler 110, through the sorter 240, through the inflow lumen 155, and into the peritoneal cavity 190. While the outflow pump(s) Pₒ 260 and the inflow pump(s) Pᵢ 265 are illustrated between the recycler 110 and the sorter 240, it should be understood that the position(s) of the outflow pump(s) Pₒ 260 and/or the inflow pump(s) Pᵢ 265 in the peritoneal dialysis system 105 can differ. For instance, in some examples, the outflow pump(s) Pₒ 260 can be co-located with the outflow sensor(s) S_{B} 235. In some examples, the inflow pump(s) Pᵢ 265 can be co-located with the inflow sensor(s) S_{A} 230. In some examples, the outflow pump(s) Pₒ 260 and/or the inflow pump(s) Pᵢ 265 can be part of the recycler 110, the sorter 240, or both, or neither. In some examples, the peritoneal dialysis system 105 can have multiple outflow pumps Pₒ 260 and/or inflow pumps Pᵢ 265. In some examples, the peritoneal dialysis system 105 can have a single pump that functions as both the outflow pumps Pₒ 260 and the inflow pumps Pᵢ 265. For instance, because the flow of fluid (e.g., the fluid 185, then the filtered fluid, then the dialysate) within the peritoneal dialysis system 105 is generally counter-clockwise (in the orientation illustrated in FIGS. 1-5), a single pump can both pull fluid (e.g., fluid 185) from the peritoneal cavity 190 through the outflow lumen 150 and push fluid (e.g., dialysate) from the peritoneal dialysis system 105 through the inflow lumen 155 into the peritoneal cavity 190. In some examples, the peritoneal dialysis system 105 may have one pump per lumen to provide more nuanced control over each lumen, as illustrated in FIGS. 2A, 2B, and 5.

In a third illustrative example, the first illustrative example and second illustrative example discussed above can be combined in a closed circuit in the peritoneal dialysis system 105. The closed circuit decreases chances of infection. The peritoneal dialysis system 105 can include the sorter 240 with the valves (e.g., valve V_{A} 205, valve V_{B} 210, valve V_{C} 215, and valve V_{D}) 220) discussed further below. While the peritoneal dialysis system 105 is in the divided inflow/outflow mode, the sorter 240 (e.g., controlled by the controller 160) can use the pumps (e.g., the outflow pumps Pₒ 260 and/or the inflow pumps Pᵢ 265) and/or the valves to direct the fluid 185 to be received (through the outflow lumen 150) from the peritoneal cavity 190 to the recycler 110 to be processed (e.g., recycled) to generate dialysate. While the peritoneal dialysis system 105 is in the divided inflow/outflow mode, the sorter 240 (e.g., controlled by the controller 160) can use the pumps (e.g., the outflow pumps Pₒ 260 and/or the inflow pumps Pᵢ 265) and/or the valves to direct the dialysate that is generated by the recycler 110 to be provided through the inflow lumen 155 to the peritoneal cavity 190. While the peritoneal dialysis system 105 is in the multifunctional lumen mode, the sorter 240 (e.g., controlled by the controller 160) can use the pumps (e.g., the outflow pumps Pₒ 260 and/or the inflow pumps Pᵢ 265) and/or the valves to direct the fluid 185 to be received through either or both lumens from the peritoneal cavity 190 to the recycler 110 to be processed (e.g., recycled) to generate dialysate. While the peritoneal dialysis system 105 is in the multifunctional lumen mode, the sorter 240 (e.g., controlled by the controller 160) can use the pumps (e.g., the outflow pumps Pₒ 260 and/or the inflow pumps Pᵢ 265) and/or the valves to direct the dialysate that is generated by the recycler 110 to be provided through either or both lumens to the peritoneal cavity 190.

In a fourth illustrative example, the first illustrative example, the second illustrative example, and the third illustrative example discussed above can be combined. As discussed above, the peritoneal dialysis system 105 may include the controller 160, which can be, or can include, a computing system 1600 (or one or more components thereof), a processor 1610, or vice versa. In some examples, operating software logic can run on the controller 160. In some examples, the controller 160 can receive sensor data from various sensors, such as the sensor(s) 135, the first lumen sensor(s) 270, the second lumen sensor(s) 275, sensor(s) S_{A} 230 of the sorter 240, sensor(s) S_{B} 235 of the sorter 240, or a combination thereof. In some examples, the controller 160 can control the pumps (e.g., the outflow pumps Pₒ 260 and/or the inflow pumps Pᵢ 265) and/or the valves (e.g., valve V_{A} 205, valve V_{B} 210, valve V_{C} 215, and valve V_{D} 220) to route the fluid 185 (received from the peritoneal cavity 190) from the outflow lumen 150 to the recycler 110, and/or to route dialysate made by the recycler 110 to the inflow lumen 155 (to the peritoneal cavity 190).

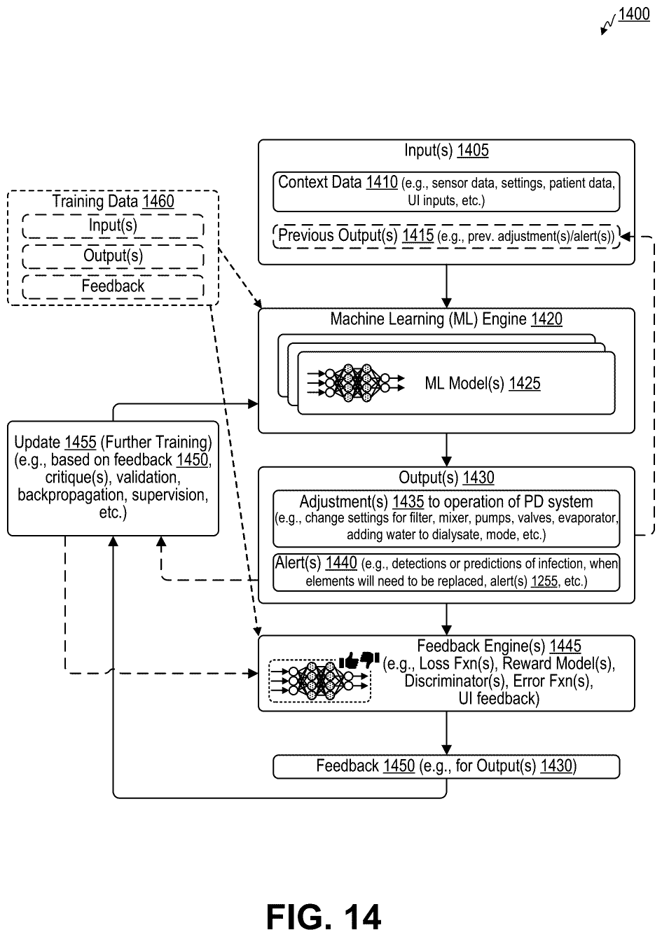

In some examples, the controller 160 can process contextual data (e.g., contextual data 1410) and/or previous adjustment(s) (e.g., previous output(s) 1415) to the peritoneal dialysis system 105 to identify adjustment(s) (e.g., adjustment(s) 1435) to make to the peritoneal dialysis system 105 (e.g., to improve performance of the peritoneal dialysis system 105 for the patient 195). The contextual data can include, for instance, the sensor data from these sensors (and/or other sensors of the peritoneal cavity 190), settings of the peritoneal dialysis system 105, patient data about the patient 195 (e.g., patient demographic data, patient medical records, patient laboratory data, clinical notes, clinical findings, imaging reports, physician examination data (e.g., temperature, weight, diagnoses, notes), patient history, patient symptoms, diagnoses, patient biopsy results, impedance or a combination thereof), user interface input(s) (e.g., input by the patient 195, a physician, a doctor, a registered nurse (RN), a nurse practitioner (NP), a family member of the patient 195, a technician, a maintenance worker, an operator of the peritoneal dialysis system 105, or another user), or a combination thereof. For instance, in some examples, the controller 160 can include, and/or use, one or more artificial intelligence (AI) algorithm(s), such as trained machine learning (ML) model(s) (e.g., ML model(s) 1425), to process the contextual data and/or the previous adjustments to identify the adjustment(s) to make to the peritoneal dialysis system 105. Examples of the controller 160 using ML model(s) (e.g., ML model(s) 1425) to process the contextual data and/or the previous adjustments to identify the adjustment(s) to make to the peritoneal dialysis system 105 are further described in FIG. 14. Data from a patient undergoing treatment, as data from other patients undergoing treatments, can be used to tune, train and adjust AI algorithm(s), for instance as in the context data 1410 and/or the training data 1460 with respect to the machine learning system 1400. The peritoneal dialysis system 105 is capable of communication through internet, Bluetooth, infrared or otherwise with other systems treating other patients, directly, or indirectly via servers, clouds and clients, running appropriate software. AI algorithms or models (e.g., ML model(s) 1425) may be trained and run on the units carried by patients, or servers, clouds, and/or clients.

The controller 160 can use logic, physics, math, and/or data mining results in addition to, and/or with, the AI algorithm(s) and/or trained ML model(s) to identify the adjustment(s). In some examples, the controller 160 can use AI algorithm(s), trained ML model(s), logic, physics, math, and/or data mining results that are trained and/or derived from historical data determined through operation of the peritoneal dialysis system 105 and/or other peritoneal dialysis systems, such as any of the types of contextual data discussed above, along with results of the peritoneal dialysis, any adjustments made to the peritoneal dialysis systems, or a combination thereof. In some examples, this can be entered manually, or downloaded, uploaded, pulled from a source, like short term, long term memory, computer network, both wireless and not.

In some examples, the controller 160 can train the AI algorithm(s) and/or trained ML model(s) to identify adjustment(s) that optimize patients' clinical outcomes, for instance to decrease chances of peritoneal infection, to optimize (e.g., minimize and/or decrease) use of energy, to optimize the longevity of filtration (e.g., maximize and/or improve how long the filter lasts to minimize and/or reduce frequency of replacing the filter), to optimize (e.g., decrease or increase depending on predicted or determined needs) the volume of rejected fluid or amount of rejected substances by reverse osmosis systems, to optimize (e.g., minimize or maximize depending on predicted or determined needs) the amount of ultra-filtration (removed fluid) during peritoneal dialysis, to optimize (e.g., maximize) the amount of clearance of waste products by the recycler 110. These adjustments and optimizations, for instance to the control that the controller 160 has over the recycler 110 (e.g., filter(s) 120, mixer 130, and/or sensor(s) 135), the sorter 240 (e.g., sensor(s) and/or valve(s) and/or pump(s)), and/or valve(s) (not shown) that control exhaust from the filter(s) 120 to the waste container 125, can optimize operation of the peritoneal dialysis system 105. The components of the peritoneal dialysis system 105 can be powered by electrical power from the power supply 170, which itself can be charged using the charger 175. In some examples, the charger 175 charges the power supply 170 using inductive charging.

In some examples, the peritoneal dialysis system 105 can operate without the recycler 110, for instance using pre-made dialysate, and can still provide improvements over traditional peritoneal dialysis systems based on improved clearance and shorter time needed for the patient 195 to be on dialysis. However, use of the recycler 110 with the peritoneal dialysis system 105 provides these benefits and more, such as continuous dialysis over long periods of time (e.g., one or more months), closed circuit operation, reduced risk of infection and therefore improved safety, and more.

As discussed above, the peritoneal dialysis system 105 includes a catheter 165 with multiple lumens, with at least one lumen being designated (by the controller 160) as an outflow lumen 150 that withdraws fluid 185 from the peritoneal cavity 190, and at least one lumen being designated (by the controller 160) as an inflow lumen 155 that provides dialysate to the peritoneal cavity 190. The lumens may be referred to as catheters themselves, in some cases. In some examples, the lumens can be at least partially flexible, at least partially rigid, at least partially straight, at least partially curved, or a combination thereof. In some examples, the position(s) of the lumens can be adjusted manually so that the tips of the lumens move within the peritoneal cavity 190. In some examples, the position(s) of the lumens can be adjusted automatically (e.g. via the controller 160 activating motor(s) or other actuator(s)) to cause the tips of the lumens move within the peritoneal cavity 190.

As noted above, the lumens include lumen sensors (e.g., first lumen sensor(s) 270, second lumen sensor(s) 275) coupled to or near the tips of the lumens. In some cases, the lumens include wires that convey power to the lumen sensors from the power supply 170, and/or that convey sensor data from the lumen sensors to the controller 160. In some examples, the sensors at the tip of the lumens (e.g., first lumen sensor(s) 270, second lumen sensor(s) 275) can communicate wirelessly with the controller 160, and/or can be powered wirelessly using induction power delivery (e.g., from the power supply 170 and/or the charger 175), for instance being charged by induction (e.g., via the charger 175). This can reduce risk of infection. The hydrostatic pressure sensors used to determine vertical relative position of tips of the lumens and their relation to ground and/or the direction of gravity, in some examples, can be located outside of the patient, but hydrostatic pressure around the lumens' tips (e.g., measured using the lumen sensors), can still be compared and used to determine the most lumen with most dependent (lowest in relation to earth) tip. The controller 160 receives sensor data from the lumen sensors. The controller 160 analyzes the sensor data from the lumen sensors (and/or from the sensor(s) of the sorter 240) to estimate the relative position(s) of the lumens' openings (or tips), for instance to estimate the lumens' respective heights relative to the ground, relative to the floor, relative to sea level, relative to the direction of gravity, and/or relative to another position or plane in three-dimensional space, or along with other data recorded by other sensors gauge the necessary flow rate of the fluid within the one or more lumens of the catheter.

In some examples, at least one of the lumen sensors (e.g., first lumen sensor(s) 270, second lumen sensor(s) 275) can include a pH sensor, a temperature sensor (e.g., thermometer, thermistor, temperature transducer), a conductivity sensor, a particulate sensor, any of the other sensor types discussed herein, or a combination thereof.

The pH measurements, temperature measurements, conductivity measurements, and/or particulate measurements of the inside of the peritoneal cavity 190 by the pH sensor can be analyzed by the controller 160 (along with other pH, temperature, conductivity, and/or particulate measurements and/or other sensor data from the sensor(s) 135, the sensor(s) S_{A} 230, the sensor(s) S_{B} 235, the first lumen sensor(s) 270, and/or the second lumen sensor(s) 275) to detect or predict signs of an infection before the infection spreads or becomes serious. For instance, in some examples, pH, temperature, conductivity, and/or concentration of certain substances (e.g., particulates, solutes, chemicals) can rise before clinical signs of infection appear. Thus, detection of an increase in pH, temperature, conductivity, and/or concentration of certain substances (e.g., particulates, solutes, chemicals) within the peritoneal cavity 190 by at least a threshold amount can predict or suggest an infection. In some examples, the controller 160 can output an alert to the patient 195 to notify or alert the patient 195 about the signs infection, for instance by sending the alert (e.g., alert 1355) to a user device (e.g., user device 1350) of a patient 195 (e.g., patient 1325) via a communication interface (e.g., communications interface 1640) of the peritoneal dialysis system 105 and/or by outputting the alert through an output device (e.g., output device 1635) of the peritoneal dialysis system 105 (e.g., displaying the alert through a display of the peritoneal dialysis system 105, playing audio corresponding to the alert through a speaker of the peritoneal dialysis system 105, actuating a haptic actuator to vibrate at least one component of the peritoneal dialysis system 105). In some examples, the peritoneal dialysis system 105 can automatically detect and/or treat (e.g., medicate, close up) an infection, for instance using by adding medication(s) (e.g., antibiotics) to the dialysate using the mixer 130 as determined using the controller 160 and/or the machine learning system 1400.

In some examples, the controller 160 can analyze the measurements of pH, temperature, conductivity, and/or concentration of certain substances (e.g., particulates, solutes, chemicals), and/or other sensor data (and/or other context data 1410) to estimate the amount of clearance provided by the catheter 165, to evaluate the quality of recycled dialysate generated by the recycler 110, to detect or predict catheter malfunction, to gauge whether the flow rate of the fluid within the one or more lumens of the catheter meets or exceeds predetermined minimum flow threshold(s) (e.g., to provide desired ultra-filtration and/or clearance), to detect whether the patient 195 is drinking at least a threshold amount of water (e.g., enough water to remain healthy and keep the peritoneal dialysis system 105 operating optimally), or a combination thereof. In some examples, the controller 160 can analyze the measurements of pH, temperature, conductivity, and/or concentration of certain substances (e.g., particulates, solutes, chemicals), and/or other sensor data (and/or other context data 1410) using one or more AI models and/or trained ML models (e.g., ML model(s) 1425) to detect and/or predict signs of infection, to estimate the amount of clearance provided by the catheter 165, to evaluate the quality of recycled dialysate generated by the recycler 110, to detect or predict catheter malfunction, to gauge whether the flow rate of the fluid within the one or more lumens of the catheter meets or exceeds predetermined minimum flow threshold(s) (e.g., to provide desired ultra-filtration and/or clearance), to detect whether the patient 195 is drinking at least a threshold amount of water (e.g., enough water to remain healthy and keep the peritoneal dialysis system 105 operating optimally), or a combination thereof (e.g., as the alert(s) 1440 and/or as adjustment(s) 1435 to correct some of these issues). In some examples, the controller 160 can output an alert to the patient 195 to notify or alert the patient 195 about any of these conditions, for instance by sending the alert (e.g., alert 1355) to a user device (e.g., user device 1350) of a patient 195 (e.g., patient 1325) via a communication interface (e.g., communications interface 1640) of the peritoneal dialysis system 105 and/or by outputting the alert through an output device (e.g., output device 1635) of the peritoneal dialysis system 105 (e.g., displaying the alert through a display of the peritoneal dialysis system 105, playing audio corresponding to the alert through a speaker of the peritoneal dialysis system 105, actuating a haptic actuator to vibrate at least one component of the peritoneal dialysis system 105).

In some examples, sensor data from chemical concentration sensors and/or particulate sensors in the sensors of the peritoneal dialysis system 105 (e.g., the sensor(s) 135, the sensor(s) S_{A} 230, the sensor(s) S_{B} 235, the first lumen sensor(s) 270, the second lumen sensor(s) 275, sensor(s) of the filter(s) 120, and/or other sensors discussed herein) can by analyzed by the controller 160 (e.g., by the ML model(s) 1425) to gauge whether an appropriate dose (e.g., above a minimum threshold and/or below a maximum threshold) of a medication, such as an anti-fibrotic substance (e.g., heparin), has been given to the patient, for instance intra-peritoneally (e.g., into the peritoneal cavity 190), into the peritoneal dialysis system 105 via the mixer 130, directly into the tubing of the peritoneal dialysis system 105 (e.g., into the lumens and/or catheter 165 and/or internal fluid channels), or a combination thereof. In some examples, sensor data from chemical concentration sensors and/or particulate sensors in the sensors of the peritoneal dialysis system 105 (e.g., the sensor(s) 135, the sensor(s) S_{A} 230, the sensor(s) S_{B} 235, the first lumen sensor(s) 270, the second lumen sensor(s) 275, sensor(s) of the filter(s) 120, and/or other sensors discussed herein) can by analyzed by the controller 160 (e.g., by the ML model(s) 1425) to identify a syndrome introduced flow rate of the fluid within the one or more lumens of the catheter, to detect need to replace filters within the filter(s) 120, to detect a need to replace substances (e.g., electrolytes or other substances) that the mixer 130 adds into the dialysate, or a combination thereof.

In some examples, the peritoneal dialysis system 105 is configured to support continuous or semi-continuous flow peritoneal dialysis via a multiple-lumen peritoneal catheter. In some examples, in doing so, the peritoneal dialysis system 105 can provide improved clearance, reduced amount of delivered glucose (less chances of worsening diabetes, or insulin resistance, less chances of infection and less trauma to PM caused by high concentration of glucose in dialysate), or a combination thereof.

In traditional peritoneal dialysis catheter tips are generally not attached to the peritoneum, and are free to travel within the peritoneal cavity 190. In some examples, the tips of the lumens (e.g., the first lumen 250 and/or the second lumen 255) of the peritoneal dialysis system 105 can similarly not be attached to the peritoneum, and can be and are free to travel within the peritoneal cavity 190. In some examples, the tips of the lumens (e.g., the first lumen 250 and/or the second lumen 255) of the peritoneal dialysis system 105 can be coupled (e.g., attached, affixed) to at least a portion of the peritoneum (e.g., at an organ and/or at a wall of the peritoneal cavity 190) (e.g., via an adhesive and/or a fastener), and can therefore be fixed relative to at least that portion of the peritoneal cavity 190.

In some examples, PD catheters and/or lumens may be directed in a specific way during insertion (e.g., to keep the lumen tip in the lower part of pelvis), and sometimes we are able to keep lumens' tips (usually only one tip actually, the one in the lowest part of the pelvis). After insertion, the tips (especially if they are anywhere other than the lower part of the pelvis) will can move within the peritoneal cavity 190, for instance depending on weight gain, weight loss, food intake, and/or the pose of the patient 195. Pose can refer to position (e.g., latitude, longitude, altitude or elevation) and/or orientation (e.g., pitch, yaw, and/or roll).

Fluid in the peritoneal cavity 190 (e.g., fluid 185, dialysate, SD, and/or PF) runs downward (e.g., in the direction of gravity) due to gravity. The peritoneal cavity 190 includes bowel loops that divide the peritoneal cavity 190 into compartments. The flow between compartments happens with the gravitation, and fluid (e.g., fluid 185, dialysate, SD, and/or PF) seeks the local minimum height to be in based on gravity and the compartments.

FIG. 3 is a block diagram illustrating an example of a use case 300 of a peritoneal dialysis system 105 that includes a lumen 350 that can be used for inflow and outflow. The peritoneal dialysis system 105 of FIG. 3 can operate in the multifunctional lumen mode discussed herein, in that the peritoneal dialysis system 105 both receives the fluid 185 from the peritoneal cavity 190 through the lumen 350 and provides the dialysate (that was generated by the recycler 110 by recycling the fluid 185) to the peritoneal cavity 190 through the same lumen 350. In some examples, the peritoneal dialysis system 105 of FIG. 3 includes the lumen 350 as the sole lumen used for both inflow and outflow.

The peritoneal dialysis system 105 of FIG. 3 may include a sorter 340 with a valve V_{B} 310 that controls outflow of fluid 185 (e.g., PF, spent dialysate) from the peritoneal cavity 190 to the recycler 110. The peritoneal dialysis system 105 of FIG. 3 may include a sorter 340 with a valve V_{A} 305 that controls inflow of dialysate (e.g., dialysate 790) from the recycler 110 to the peritoneal cavity 190. The peritoneal dialysis system 105 of FIG. 3 may include a sensor(s) S_{A} 330 and/or a sensor(s) S_{B} 335, which may include similar types of sensors as discussed with respect to the sensor(s) S_{A} 230 and/or the sensor(s) S_{B} 235, and may measure similar types of sensor data as discussed with respect to the sensor(s) S_{A} 230 and/or the sensor(s) S_{B} 235.

In some examples, in a peritoneal dialysis system 105 with a single-lumen catheter (e.g., as in the peritoneal dialysis system 105 of FIG. 3), a predetermined volume of dialysate is provided as inflow to the peritoneal cavity 190 through the lumen (e.g., lumen 350) and remains in the peritoneal cavity 190 for a predetermined amount of time (e.g., one or more hours, such as 2-4 hours). For instance, the predetermined volume of dialysate can be 2000-3000 cc.

The spent dialysate can then be drained (e.g., as the fluid 185) as outflow via the same lumen 350. So, if the fluid 185 was filling the peritoneal pocket it was in, there is a high likelihood to retrieve at least a large part of that fluid 185 the predetermined amount of time later with the draining. Whatever fluid 185 is not retrieved as outflow by the lumen 350 can, in some cases, go into other peritoneal pockets, fill them, and stay there, until the patient 195 changes position. In the meantime, the lumen 350 can drain fluid 185 from other pockets fully or almost fully. In some examples, all the pockets the fluid 185 could have gone to are already full of dialysate from prior fills, therefore a new fill of fluid 185 stays within the drainage area of the lumen 350.

In some examples, the lumen 350 of FIG. 3 can include lumen sensor(s) 370 at the tip of the lumen 350. The lumen sensor(s) 370 can be used (e.g., by the controller 160) to determine whether the tip of the lumen 350 is in the fluid 185 or not, for instance to know whether or not to stop providing negative pressure (suction) for outflow of fluid 185 through the lumen 350. In some examples, the lumen sensor(s) 370 can be similar to the first lumen sensor(s) 270, the second lumen sensor(s) 275, and/or other lumen sensor(s) discussed herein.

In order to use a catheter 165 with at least two lumens (as in the peritoneal dialysis system 105 of FIG. 1, FIGS. 2A-2B, and/or FIGS. 4-5) for continuous or semi continuous peritoneal dialysis, at least one lumen serves as an inflow catheter or lumen, and at least one lumen serves as an outflow catheter or lumen. In some examples, only the lumen(s) with the highest pressure and/or that are the most dependent (e.g., lumen whose tip has the lowest height above sea level in the peritoneal cavity 190) is designated (e.g., by the controller 160) to be an outflow lumen, to ensure that as much of the fluid that is infused into the peritoneal cavity 190 via the inflow lumen(s) as possible is later retrieved by the outflow lumen(s). On the other hand, only the lumen(s) with the lowest pressure and/or that are the least dependent (e.g., lumen whose tip has the highest height above sea level in the peritoneal cavity 190) is designated (e.g., by the controller 160) be an inflow lumen, to ensure that as much of the fluid that is infused into the peritoneal cavity 190 via the inflow lumen(s) as possible is later retrieved by the outflow lumen(s).

In some examples, the recycler 110 of the peritoneal dialysis system 105 of FIG. 3 can include a buffer tank for fluid that allows the recycler 110 to still recycle fluid to generate dialysate. The buffer tank can temporarily store fluid 185 (e.g., spent dialysate) received from the peritoneal cavity 190 through the 350, can temporarily store filtered fluid filtered by the filter(s) 120, can temporarily store dialysate generated by the mixer 130 and/or verified by the sensor(s) 135, or a combination thereof. For instance, in some examples, in the multifunctional lumen mode, the dialysate provided into the peritoneal cavity 190 through the lumen 350 can remain in the peritoneal cavity 190 for a period of time (e.g., several hours) before the lumen 350 retrieves the spent dialysate (e.g., as fluid 185), for instance to ensure that the dialysate absorbs waste from the peritoneal cavity 190. Similarly, in some examples, the peritoneal dialysis system 105 can wait before providing the dialysate, for instance storing the dialysate in the buffer tank until the patient fluid 185 is ready to receive the dialysate into the peritoneal cavity 190.

FIG. 4 is a block diagram illustrating an example of a use case 400 of a peritoneal dialysis system 105 that includes two inflow lumens (e.g., a first lumen 450 and a second lumen 455) and an outflow lumen (e.g., a third lumen 460), each with corresponding lumen sensors (e.g., sensor(s) S_{A} 405 at the base of the first lumen 450, first lumen sensor(s) 470 at the tip of the first lumen 450, sensor(s) S_{B} 410 at the base of the second lumen 455, second lumen sensor(s) 475 at the tip of the second lumen 455, sensor(s) S_{C} 415 at the base of the third lumen 460, and third lumen sensor(s) 480 at the tip of the third lumen 460) and corresponding sensors in a sorter 440. The sensors in the sorter 440 include sensor(s) S_{A} 405 that measure characteristics of the dialysate flowing to the peritoneal cavity 190 through the first lumen 450, sensor(s) S_{B} 410 that measure characteristics of the dialysate flowing to the peritoneal cavity 190 through the second lumen 455, and sensor(s) S_{C} 415 that measure characteristics of the fluid 185 retrieved from the peritoneal cavity 190 through the third lumen 460. In some examples, the lumens can have different lengths compared to one another. For instance, the second lumen 455 is illustrated as being the shortest lumen in FIG. 4, with the first lumen 450 being longer than the second lumen 455, and the third lumen 460 being longer than the first lumen 450. In some examples, the different lengths of the lumens can help to ensure that the tips of the different lumens remain at different positions (relative to one another) within the peritoneal cavity 190. In some examples, two or more of the lumens of a peritoneal dialysis system 105 can have the same lengths.

In some examples, hydrostatic pressure sensors can be positioned outside of patient 195 (e.g., the sensor(s) S_{A} 405, sensor(s) S_{B} 410, and/or sensor(s) S_{C} 415). In some examples, placing pressure sensors within the abdomen (e.g., as in the lumen tip sensors) can be risky, as bowels can push on them as they move. Thus, checking pressures via sensors that are outside of the patients can be beneficial. In some examples, the controller 160 can calculate a moving average, minimum, or maximum for a given pressure sensor, and use this calculation as the pressure value captured by that sensor in the determination of which lumen is dependent.

While the second lumen 455 is designated as an inflow lumen in FIG. 4, in some examples, the second lumen 455 can be designated as a second outflow lumen instead. In some examples, having more than one outflow lumen (as in a modified variant of FIG. 4 and/or as in FIG. 5) can improve dialysate saturation in the peritoneal cavity 190 and/or clearance of the fluid 185 from the peritoneal cavity 190. In some examples, having more than one outflow lumen can help extract fluid 185 from different pockets or chambers of the peritoneal cavity 190, for instance to help retrieve fluid 185 from between various organs in the peritoneal cavity 190 that a single outflow lumen might not reach. In a peritoneal dialysis system 105 that has more than one outflow lumen, the lumens with the most dependent tips are designated as outflow lumens. Thus, having two or more lumens allows for continuous peritoneal dialysis.

Similarly, the lumen(s) with the highest elevation (from the level of the sea or ground) is designated (e.g., by the controller 160) to be inflow lumen(s). Having more than one inflow lumen in the peritoneal dialysis system 105, as in FIGS. 4-5, can improve continuous peritoneal dialysis. For instance, at entry points (e.g., at the tips of the inflow lumen(s)), dialysate is totally unsaturated with waste. The more entry points of dialysate into the peritoneal cavity 190, the more variety of paths, and the greater distance (and thus longer duration of time) the dialysate travels from inflow entry points (e.g., the tips of the inflow lumen(s)) to exit points (e.g., the tips of the outflow lumen(s)). Increasing the variety of paths traveled by the dialysate, and/or the distance and/or time that the dialysate travels from entry points to exit points, helps extract more waste (e.g., solutes) from different parts of the peritoneal cavity 190. Since the peritoneal dialysis system 105 optimally uses every drop of dialysate flowing through the peritoneal cavity 190 and make the dialysate as saturated with waste solutes as possible (especially when the dialysate is spent, but also when it is recycled), the peritoneal dialysis system 105 can adjust inflow rate to cause the dialysate to get saturated before the dialysate reaches the proximity of outflow lumen tip. Thus, there is extra benefit from making the dialysate travel for few more centimeters from second most dependent lumen tip to most dependent lumen tip). Thus, having more than inflow lumen and/or more than inflow lumen can improve extraction of waste from the peritoneal cavity 190 using the dialysate.

The outflow lumens 550 and inflow lumens 555 of FIG. 5 also include lumen sensors. The lumen sensors of the outflow lumens 550 and inflow lumens 555 can be similar to the first lumen sensor(s) 270, the second lumen sensor(s) 275, the lumen sensor(s) 370, the first lumen sensor(s) 470, the second lumen sensor(s) 475, the third lumen sensor(s) 480, or a combination thereof. The lumen sensors of FIG. 5 are labeled LS_{A} (for the lumen sensor(s) of lumen L_{A}), LS_{B} (for the lumen sensor(s) of lumen L_{B}), LS_{C} (for the lumen sensor(s) of lumen L_{C}), and LS_{D} (for the lumen sensor(s) of lumen L_{D}).

While the internal valves of the sorter 440 (FIG. 4.) are not shown, it should be understood that sorter 440 can include internal valves in a similar arrangement and/or configuration to the arrangement and/or configuration of valves illustrated in FIGS. 2A-2B, but allowing any of the three lumens to change between behaving as an inflow lumen and behaving as an outflow lumen at any time as needed, for instance if the patient 195 changes position (e.g., lies down, stands up, etc.).

Attempting to use a lumen whose lumen sensor detects a high pressure (e.g., the most dependent lumen(s)) as an inflow lumen would necessitate pumping up of dialysate fluid all the way to the level (e.g., against hydrostatic pressure) to be high enough until it reaches the tip(s) of other lumen(s)—the outflow lumen(s). This would reduce flexibility to adjust the amount of residual peritoneal dialysate (RPD) in peritoneum. In a scenario like this (where the dependent lumen is used for inflow), infusing dialysate via a dependent lumen until the dialysate level reaches the tip(s) of outflow lumen(s) peritoneal cavity 190 can cause problems. For instance, if a patient is unlucky, and the tip(s) of the outflow lumen(s) end up at a highest point in the peritoneal cavity 190 (e.g., near the patient's diaphragm), the outflow lumen(s) would need to fill the entire peritoneal cavity 190 with dialysate (e.g., to around 4000 cc of RPD) for any dialysate to be evacuated from the peritoneal cavity 190 via the outflow lumen(s). This could cause complications, like constipation, high urinary frequency, high stool frequency, stomach pain, and more.

Similarly, using lumens with low pressure (e.g., the lumens whose tips are least dependent and thus highest above the ground) as outflow lumens through which fluid 185 is retrieved from the peritoneal cavity 190 can cause problems. Without any negative pressure (e.g., suction) applied, such non-dependent outflow lumens can fail to receive any outflow of fluid 185. With minimal negative pressure (e.g., suction) applied, such non-dependent outflow lumens can receive minimal outflow-insufficient for maintaining continuous outflow. With strong negative pressure (e.g., suction) applied, such non-dependent outflow lumens can risk sucking in some bowel loops into the lumen's tips, clogging the outflow lumens' tips with the loops and potentially damaging the bowel loops. With strong negative pressure (e.g., suction) applied, such non-dependent outflow lumens can risk life-threatening complications (e.g. bowel perforations, peritoneal bleeding).

On the other hand, use of dependent lumens as outflow lumens, and non-dependent lumens as inflow lumens, overcomes these issues, providing improved peritoneal dialysis that can be run continuously. For instance, because gravity helps guide the dialysate through the peritoneal cavity 190 from the entry point(s) (e.g., the tip(s) of the inflow lumen(s)) to the exit point(s) (e.g., the tip(s) of the outflow lumen(s)), gravity can in some cases also help guide the spent dialysate (e.g., the fluid 185) at least partially through the outflow lumen(s) and to the rest of the peritoneal dialysis system 105, minimizing how much negative pressure the pump(s) of the peritoneal dialysis system 105 need apply (if any) to the outflow lumen(s). This can reduce power consumption of the peritoneal dialysis system 105 in addition to enabling continuous peritoneal dialysis, improving clearance of waste from the peritoneal cavity 190, and reducing risk of injury. Further, the inflow lumens can provide less dialysate (e.g., around 500 cc of RPD) to clear waste from the peritoneal cavity 190.

In some examples, pressure registered by lumens (e.g., hydrostatic pressure) with the vertical relation between lumen tips can change depending on the patient's pose (e.g., whether patient lies down on left side, back, belly, on the right side, sits, or stands up), movement(s) of the lumen tip(s) and/or catheter (e.g., when patient is moving, or when patient gains or loses weight, or when patient has a bowel movement, or when the patient cats or drinks). Additionally, availability for the fluid to be drained by the outflow lumen for a given pocket of peritoneal fluid (e.g., whether the lumen should be designated as an outflow lumen) can depend on how high the pressure is at that lumen pressure sensor. Generally, the higher the pressure at the tip of the lumen (as measured by a lumen sensor), the deeper (more dependent) the tip of the lumen is, indicating that the lumen should be designated as an outflow lumen. In contrast, if there is lower pressure at the lumen sensor of a specific lumen (than at another lumen sensor of another lumen), the peritoneal dialysis system 105 can designate the specific lumen to be an inflow lumen, to be use for inflow (e.g., infusion, influx) of dialysate.

Thus, to determine which lumen(s) are to be designated to be outflow lumens (e.g., highest pressure(s)) and which lumen(s) are to be designated to be inflow lumens (e.g., at the lowest pressure(s)), data from lumen sensors is used (e.g., pressure or another indication of height of the lumens' tips). The height can be height above sea level and/or ground, for instance along an axis parallel to the direction of gravity. This can allow for the peritoneal dialysis system 105 to provide continuous or semi-continuous peritoneal dialysis, ultimately providing increased clearance, lower RPD with at least two lumen catheters. This can also allow the peritoneal dialysis system 105 to be portable, allowing the patient fluid 185 to live a higher quality of life.

To determine the pressure (e.g., hydrostatic pressure) (e.g., mostly related to vertical relation (height from the ground)) of the lumens in real-time, a few different types of sensors can be used, individually or in combination. Several illustrative examples are discussed below. These illustrative examples can be combined.

In a first illustrative example, the peritoneal dialysis system 105 can use pressure interception to determine which lumens are dependent (lower in the direction of gravity) and which are non-dependent (higher, opposite the direction of gravity) relative to one another. In pressure interception, the peritoneal dialysis system 105 samples, in real-time or near real-time (e.g., every 20 seconds, 40 seconds, 1, 3, 5, 10, 60, 120 minutes or more time) pressures transduced by sensors coupled to the peritoneal dialysis system 105 (e.g., located outside of patient 195 and/or inside of the patient 195), and samples pressures transduced by lumen sensors at the lumens (e.g., at the tips of the lumens). In some examples, the peritoneal dialysis system 105 can evaluate these pressures after the fluid flows are stopped or close to stopped, and/or are stable for at least a predetermined amount of time. The peritoneal dialysis system 105 can average the pressures over one or more recent timing periods (e.g., 1, 2, 3, 4, or 5 or more last timing periods). The lumen with highest average pressure P (e.g., higher than minimal, maximal, and/or average of pressures measured in other lumens) by the margin of P*M (e.g., where M is found empirically, and in some cases can be between 0.0 to 0.2) can be designated as the outflow lumen (e.g., with the highest pressure indicating that this is the most dependent lumen). The timing and periods can vary for different activities of the patient, such as walking, sleeping, sitting, exercising, and the like, to improve responsiveness without unnecessary sampling (e.g., which can stop the flow in some embodiments and can thus impede dialysis). In some examples, the pressure can be checked by the transducers at the tips of the lumens and/or at sensors outside of the patient 195 (e.g., at the base of the lumen(s), in the sorter, or the like) . . . . In some embodiments (e.g., with lumen sensors), stoppage of the flow is not required, and continuous flow (e.g., of dialysate and fluid 185) can be maintained even while the sensors continue to periodically check which lumen(s) are dependent and which lumen(s) are non-dependent (e.g., in case that changes due to movement of the lumen(s) and/or change(s) to the pose of the patient 195).

In some examples, in a stable condition, when (a) there is no inflow and no outflow from peritoneal cavity 190, or sometimes (b) when inflow and outflow are at constant rate and in other conditions, the hydrostatic pressure measured by hydrostatic pressure sensors, can be the same as pressure recorded by the pressure sensors. For instance, in such conditions, in of FIG. 2A, the first lumen sensor(s) 270 can register similar pressure(s) as the sensor(s) S_{A} 230, and/or the second lumen sensor(s) 275 can register similar pressure(s) as the sensor(s) S_{B} 235.

In a second illustrative example, the peritoneal dialysis system 105 can use radio-magnetic coils, in the perpendicular plains, in the lumen sensors at the tip(s) of each of the lumens. The radio-magnetic coils can be activated by induction, and emitting their respective signal upon activation, with or without a battery, with the signal being triangulated by an outside receiver and/or analyzer (e.g., in the peritoneal dialysis system 105, for instance using the sensor(s) S_{A} 230 and/or the sensor(s) S_{B} 235 and/or the controller 160). In some examples, the outside receiver and/or analyzer can be laying on the floor, coupled to the patient 195's shoe, coupled to the patient 195's neck, coupled to the patient 195's backpack or purse, or otherwise localized in known vertical relation of lumens tips to the peritoneal cavity 190 and/or abdominal cavity.

In a third illustrative example, the patient 195 and/or another user can identify, via a user interface of the peritoneal dialysis system 105, whether the patient 195, he is sitting, standing, laying (e.g., on left side, right side, back, or belly). With some assumptions, in the case of catheters with two lumens as in FIG. 1 and FIGS. 2A-2B, the peritoneal dialysis system 105 can determine which lumen(s) are dependent and which are non-dependent based on this information from the UI. For instance, if the peritoneal dialysis system 105 knows the approximate positions of certain lumens (e.g., one lumen that has tip and/or sensor in the lower pelvis, and another lumen having tip and/or sensor just under the diaphragm, at the entry point, to the left and up or to the right and up to the umbilicus).

In a fourth example, the peritoneal dialysis system 105 can use a combination of the third example with the first and/or second example. For instance, in the fourth example, the peritoneal dialysis system 105 can perform sampling of sensor data from the sensor(s) (e.g., pressure sensor(s) and/or radio-magnetic coils) periodically, each time the patient 195 identifies via the UI that the patient's position or pose has changed in a way that may change which lumen(s) are dependent and which lumen(s) are non-dependent, that may change the direction(s) of flows (e.g., inflow vs outflow), and so forth. The sensor(s) can be resampled, and in some cases, the direction of flows (e.g., inflow vs outflow) within can be changed (e.g., certain lumens can change from being inflow lumens to being outflow lumens, and/or certain lumens can change from being outflow lumens to being inflow lumens).

In some examples, once the peritoneal dialysis system 105 detects the pressure (e.g., temporary or averaged for the time) in the lumens or at the proximity of lumens tip via transducers or other sensors (e.g., using small lumens to transduce the pressures to dialyzer) that indicate vertical position (e.g., height from the ground) of the tips of the lumens, the peritoneal dialysis system 105 can, in real-time, direct inflow of dialysate-time into the non-dependent lumen(s) (e.g., the inflow lumen(s)), and allow for outflow (e.g., with a desired pressure valve) from the dependent lumen(s) (e.g., the outflow lumen(s)).

Going back to FIG. 2A, in some examples, the second lumen sensor(s) 275 can include a temperature transducer and/or other sensor located at a tip of the second lumen 255, which has the most dependent tip of all of the lumens (between the first lumen 250 and the 255) in FIG. 2A. Thus, the second lumen 255 is the most dependent lumen (MDL) for this patient position and lumens' tips intra-peritoneal configuration, also known as the lowest-laying-tip lumen.

In this patient's position, the first lumen sensor(s) 270 can include a temperature transducer and/or other sensor located at a tip of the first lumen 250 which has the least dependent tip of all of the lumens (between the first lumen 250 and the 255) in FIG. 2A. Thus, the first lumen 250 is not the most dependent lumen (NMDL) for this patient position and lumens' tips vertical configuration, also known as not-lowest-laying-tip lumen.

Analysis of the temperature can optimize clearance. If inflow is allowed to cool (e.g., 5 C degrees cooler) dialysate, and slowly decrease flow rate and outflow rate, from max to min, the outflow dialysate temperature slowly rises as a result. At some point, the temperature stops rising once it reaches body temperature. Thus, slowing down flow rate and outflow rate below that rather would not improve clearance. This can provide real-time clearance optimization.

In some examples, the filter(s) 120, the mixer 130 and the 135 are serially connected within the recycler 110. In some examples, the filter(s) 120 include at least one reverse osmosis (RO) filter. In some examples, the filter(s) 120 include a plurality of RO filter connected serially or in parallel. The RO filter(s) of the filter(s) 120 can ensure nearly 99% rejection of electrolytes. In some examples, the 120 can include activated replaceable or permanent carbon filter(s), resin filter(s), multimedia filter(s), desalination filter(s), online centrifuge filter(s), electrolyzer(s), hot filtration filter(s), cold filtration filter(s), centrifugal filtration filter(s), gravity filtration filter(s), vacuum filtration filter(s), cross-flow filtration filter(s), nano-filtration filter(s), ultra-filtration filter(s), micro-filtration filter(s), bio-filtration filter(s), microbial adhesion filtration filter(s), column adhesion filtration filter(s), immune-adhesion filtration filter(s), sterilization filter(s), sieving filter(s), column adhesion filter(s), micro-porous adhesion filtration filter(s), adhesion filtration filter(s), other filtration methods, or a combination thereof.

The mixer 130 is a dialysate mixer that adds electrolytes to the filtered fluid (filtered by the filter(s) 120) to generate dialysate. In some examples, the mixer 130 adds glucose to the filtered fluid (filtered by the filter(s) 120) to generate dialysate. The filtered fluid may be close enough to pure water (e.g., lower than a threshold amount of solutes and/or other wastes), to make a new, fresh, and/or or regenerated dialysate (or “recycled dialysate”) at the mixer 130. The mixer 130 adds electrolytes, glucose, and/or sometimes other substances, including water, are added to water, to regenerate the dialysate. These substances are in removable cartridges and can be removed when empty. The cartridge 710, the cartridge 720, and the cartridge 730 can be examples of these cartridges. Even though whole circuit of the recycler 110 operates within closed circuit, these cartridges can be removed and replaced, if they are empty. In some examples, the mixer 130 (and/or another part of the peritoneal dialysis system 105) includes is UV light that is directed to shine at, and that shines at, the connectors (e.g., at the injector 740, the injector 745, and/or the injector 750) to prevent infection where cartridges are connecting to circuit of the embodiment.

The sensor(s) 135 can include one or more conductivity sensors, pH sensors, glucose concentration measurement sensors, flow sensors, (hydrostatic) pressure sensors, other sensors, or a combination thereof. The controller 160 can analyze the sensor data from the sensor(s) 135 to assure quality of recycled dialysate generated by the mixer 130 and to control the mixer 130, the flow rate of dialysate through lumens, the pressure used for reverse osmosis, the rejected amount from the filter(s) 120, an emergency stoppage of dialysis in case of detection of dialysate with inadequate quality (e.g., less than a threshold), alerts of low power in the power supply 170 (e.g., low battery), or a combination thereof. In some examples, the controller 160 can use the data from the sensor(s) 135 (and/or other sensors) as context data 1410 to input into AI or ML models (e.g., ML model(s) 1425) to generate adjustment(s) 1435 to optimize the flows and pressures, predict the need for change(s) to flow(s) depending on prediction(s) of the patient's pose(s) and/or pose change(s) and/or activity. The controller 160 can use this to control the rest of the peritoneal dialysis system 105.

The sorter (e.g., sorter 115, sorter 240, sorter 340, sorter 440, sorter 540) samples hydrostatic pressures from all lumens data from (hydrostatic) pressure sensors of the sorter(s) (e.g., sensor(s) S_{A} 230, the sensor(s) S_{B} 235), sensor data (e.g., pressure, temperature) from lumen sensors (e.g., at the bases and/or tips of the lumens) to find the most dependent lumen (MDL) and optimize dialysis (use of dialysate) and to deliver maximal clearance and UF with minimal dialysate regeneration. In some examples, the controller 160 can use AI and/or ML models (e.g., ML model(s) 1425), mathematics, physics, and/or logic to control the sorter, adjust settings for different valves and pumps according, and thus route the fluid 185 and/or the dialysate.

The controller 160 runs operating system logic that can control all controllable components of the peritoneal dialysis system 105, such as the valves of the sorter, the flow pumps, heaters, coolers, pressurizers, pumps, reservoirs, valves, lumens, exhausts, drains, and sensors. The controller 160 can analyze data and/or perform some functions autonomously, using artificial intelligence (AI) and machine learning (e.g., machine learning system 1400), math and physics, and pre-installed logical gates and conditions, and input from operator(s) (e.g., patient, nurse, physician). The controller 160 can communicate via wireless interface(s) (e.g., including, but not limited to via WIFI, Bluetooth, infrared, GPS, 3G, 4G, 5G, 6G) with a user device (e.g., user device 1350) of an operator and/or patient, and/or other peritoneal dialysis systems associated with other patients via their respective controllers, databases with data recorded from the peritoneal dialysis system 105 and/or from peritoneal dialysis systems of other patients, sensors, or a combination thereof.

Turning back to FIG. 4, the sorter 440 and the controller 160 sample hydrostatic pressures from all lumens, with data from hydrostatic pressure sensor of first lumen 450 (sensor(s) S_{A} 405) and the second lumen 455 (sensor(s) S_{B} 410), the hydrostatic pressure sensor of the third lumen 460 (the sensor(s) S_{C} 415), and other data from sensors, like temperature and/or pressure sensors of the lumen sensors (e.g., first lumen sensor(s) 470, second lumen sensor(s) 475, third lumen sensor(s) 480), to find the MDL and with systems software logic optimize dialysis (use of dialysate) and to deliver maximal clearance and UF with minimal needed dialysate regeneration.

In FIG. 4, after the dialysate is infused, there is indwelling, residual peritoneal dialysate (RPD) (e.g., the fluid 185). Although the tri-lumen peritoneal dialysis system 105 in FIG. 4 is more complicated than the dual-lumen peritoneal dialysis system 105 of FIG. 1 and FIGS. 2A-2B, the peritoneal dialysis system 105 of FIG. 4 provides additional benefits.

For instance, the sorter 440 and the controller 160 can optimize infusion of dialysate via first lumen 450 and second lumen 455 in such a way, to optimize (maximize) contact time (e.g., contact time-surface or contact surface) between dialysate and PM, hence, to maximize the transfer of solutes and UF between the patient and the dialysate. For that purpose, the controller 160 can analyzing signals from all temperature sensors and/or pressure sensors to determine the time the dialysate has to take to flow from first lumen 450 to third lumen 460 and from second lumen 455 to the third lumen 460. This process can be referred to herein as Flow Optimization per Inflow Lumen or (FOpIL).

In the case of first lumen 450, second lumen 455 and third lumen 460 being in vertical relation and with patient positioned as shown on FIG. 4, FOpIL includes several operations. To find the time needed for dialysate to flow from first lumen 450 to third lumen 460, the sorter and/or the controller 160 register temperatures at all temperature sampling points (third lumen sensor(s) 480, second lumen sensor(s) 475, first lumen sensor(s) 470) and/or stop any flow via second lumen 455. The sorter and/or the controller 160 infuse dialysate at the temperature: first lumen sensor(s) 470+/−delta Celsius (e.g., delta being in the range of +/−0.5-1.5 Celsius degrees) at a rate of F (e.g., F being approximately 10-40 cc/second) via the first lumen 450.

The sorter and/or the controller 160 establish (e.g., measure) time t1, needed for the temperature at the third lumen sensor(s) 480 drop to new, stable lower than initially registered temperature at the start of FOpIL. The sorter and/or the controller 160 then resume stable, equal flows via all inflow lumens with the same temperatures as earlier registered at the start of FOpIL. The sorter and/or the controller 160 can stop any flow via first lumen 450. The sorter and/or the controller 160 infuse dialysate at the temperature: second lumen sensor(s) 475+/−delta Celsius (e.g., delta being in the range of +/−0.5-1.5 Celsius degrees) at a rate of F (e.g., delta being around 10-40 cc/second) via second lumen 455.

The sorter and/or the controller 160 measure time t2, needed for the temperature at the third lumen sensor(s) 480 drop to new, stable lower than initially registered temperature at the start of FOpIL. The sorter and/or the controller 160 then resume stable, equal flows via all inflow lumens with the same temperatures as earlier registered at the start of FOpIL

The sorter and/or the controller 160 achieve normal operations, but now with times t1 and t2 determined. From now on, the amount of flow directed to first lumen 450 (F1) and the amount of flow directed to second lumen 455 (F2) maintain the relationship: F1/F2=k*(t1/t2), where k can be established experimentally for different times t1 and t2 and desired level of clearances and UF. In some examples, k=1. The more time it takes (t1) for dialysate to travel from first lumen sensor(s) 470 to third lumen sensor(s) 480, the more likely time-surface contact is higher for the dialysate infused via first lumen sensor(s) 470. In some examples, it may be beneficial to infuse more dialysate via first lumen 450 and/or the second lumen 455. Desired total flow F1+F2 can depend on desired UF, and can be used to determine k?. So for a given desired UF, the controller 160 can use this relationship to calculate optimal clearance. Likewise, for a desired clearance, the controller 160 can use this relationship to calculate an optimal UF.

Maintaining F1/F2 as equal to or close to k*(t1/t2) helps to ensure efficient use of dialysate. For example, if the time needed by dialysate to travel from the first lumen 450 to the third lumen 460 is t1 which is twice as long as t2, then during continuous flow peritoneal dialysis (CFPD), the flow F1 can be twice as high as the flow F2 due to the time needed by dialysate to travel from second lumen 455 to third lumen 460. In other words, the flow F2 is maintained as twice as low as F1, so both dialysate from first lumen 450 dialysate from second lumen 455 maintain similar solute saturation.

As in the system in FIG. 4, in a peritoneal dialysis system 105 with more than two lumens, and/or in a peritoneal dialysis system 105 with more than 1 NMDLs (n NMDLs), FOPIL can be being performed, but can involve more steps, for instance measuring of times t1, t2, . . . tn and flows F1, F2, . . . . Fn per each of NMDL1, NMDL2, . . . . NMLDn, with the controller 160 maintaining the ratio of: k*t1: t2: . . . : tn or another ratio determined in trials before mass producing the device.

Further steps can be performed (e.g. by the controller 160) after FOpIL. In some examples, FOpIL can be applied to a multi-lumen peritoneal dialysis system 105, such as the peritoneal dialysis system 105 of any of FIGS. 1-5, to optimize UF or clearance or dialysate use. For instance, such optimization can be performed by comparing and finding optimal flow F for the given patient 195, versus (historical) prior calculated and measured clearances and/or flow times, from the past for the same patient 195 and/or other patients with similar dialysis system(s) (e.g., peritoneal dialysis system 105). The patient data from the other patients can be retrieved from a database of other patients with flow F and time t values, for instance using AI models, ML models (e.g., ML model(s) 1425), and/or statistics.

For peritoneal dialysis system 105 with two or more lumens (e.g., FIG. 1, FIGS. 2A-2B, and/or FIGS. 4-5)), an optimization of use of dialysate by the sorter and/or the controller 160 can be referred to as Total Flow Optimization (TFO). TFO involves finding the optimal total flow (TF) as sum of all inflow flows (F1 . . . . F2) for all inflow lumens, in longer time period (e.g., tens of minutes, hour, hours) such that the further increase of inflow flows for all inflow lumens would not increase efficiency of dialysis (measured by the clearance per that time unit).

The TFO can be performed periodically, for instance during the maintenance of the peritoneal dialysis system 105 (for example during the visits in-person, or virtual, with provider). To perform TFO, the sorter and/or the controller 160 gradually increase TF (e.g., by increasing all flows of inflow lumens), until further increase of TF does not increase the clearance by delta KT/V (e.g., delta KT/V being approximately 1-10%).

The TFO can be performed continuously, during the use of peritoneal dialysis system 105 for continuous flow peritoneal dialysis (CFPD). To find the TF, the sorter and/or the controller 160 gradually increase TF (e.g., by increasing all flows of inflow lumens), until further increase of TF does not increase the conductivity measured by sensors (e.g., sensor(s) S_{A} 230, the sensor(s) S_{B} 235), or the difference between these conductivity is less than given deltaCO (e.g., with deltaCO being approximately 1-10%).

In some examples, for various RPD (residual peritoneal dialysate) (usually higher the better) there are different TF values. In some examples, for most RPD, higher TF provides better clearance of waste products. On the other hand, higher TF can also produce higher amounts of rejected water in the filter(s) 120 (e.g., filter subsystem 605), and cause the patient to need to drink more water.

The sorter and/or the controller 160 can also perform an Initiation process, in which the controller 160 finds a minimal RPD necessary for CFPD in each patient. Like TFO, Initiation is applicable to any peritoneal dialysis system 105 with two or more lumens. During the Initiation, in some examples, the patient 195 stays as still as reasonably can. The sorter and/or the controller 160 transduces pressures from all lumens (e.g., from lumen sensors and/or other sensors of the peritoneal dialysis system 105). If at least one lumen has an average hydrostatic pressure P sampled in 1,2,3,4,5 timing periods and averaged over 1,2,3,4,5 timing periods higher than any other lumens, by the margin of P*M (e.g., where M is approximately 0.1-0.2) the sorter and/or the controller 160 finishes Initiation, and specifies which lumen becomes the MDL. If none of the lumens meet this criteria (e.g., associated with the margin of P*M), the sorter and/or the controller 160 infuse a volume V of dialysate (e.g., V being at least 50 cc) via all lumens (e.g., or at least all inflow lumens), with gradually increasing flows from 0 cc/min to a predetermined value flow (e.g., approximately 50 cc/min), changing flow rate at a predetermined period (e.g., every 5 seconds) by a predetermined amount (e.g., 5 cc/min). The sorter and/or the controller 160 stops all inflows and waits for a predetermined time (e.g., 1-3 minutes) for fluid to travel between pockets within the peritoneal cavity 190, and (hydrostatic) pressures to achieve relatively stable levels and then returns to the start of the Initiation to repeat the process as check, with infused fluid, if by now, it is possible to determine the MDL.

In some examples, the Initiation is repeated in various conditions. For instance, the Initiation can be performed and/or repeated when requested by the patient or operator (e.g., physician, RN), when a patient changes position and/or requests Initiation to occur in order to make dialysis efficient, when the outflow stops, when the pressures analyzed during the Pressures measurement process described above (FOpIL) does not allow sorter and/or the controller 160 to find the MDL.

Another function of sorter and/or the controller 160 is to provide redundancies. In case any of the lumens becomes clogged, the sorter can allows other lumens to compensate, allowing the peritoneal dialysis system 105 to function similarly to a peritoneal dialysis system 105 that has one less lumen.

In some examples, the peritoneal dialysis system 105 can operate as a single lumen peritoneal dialysis system 105, as illustrated in FIG. 3, for instance if the other lumens are clogged or if the peritoneal dialysis system 105 cannot determine which lumen is dependent. In some examples, in a single lumen peritoneal dialysis system 105 (as illustrated in FIG. 3), the peritoneal dialysis system 105 can have a fluid residual, and/or receive, pre-made dialysate to provide into the peritoneal cavity 190.

In some examples, if changing directions and/or designations of lumens as MDL (outflow) and NMDL (inflow), if done too frequently, can decrease the efficiency of dialysis by the peritoneal dialysis system 105. For instance, used dialysate that is saturated with waste can end up dwelling in outflow lumens (e.g., between the lumen tip and the valves of the sorter), before the dialysate changes direction and returns, causing the spend dialysate to be used as inflow dialysate (e.g., which won't clear out much further waste from the body of the patient 195). A delay or lag in timing periods for sampling and/or changing of lumen configurations can reduce or eliminate the possibility of such an issue occurring. In cases like this the system should switch to fail safe dialysis, providing . . . fail safe in comments/backup dialysis, contemporary ( . . . “In other examples, when one or more lumens clog, or become unusable for another reason, controller can provide dialysis using one lumen of the catheter only, repeating sequence of operations including: infusing dialysate set volume bolus during set time, dwelling dialysate bolus in PC for set time, and draining it whole or predetermined part of it during set time. To detect these circumstances, within its program, controller can compare sensor pressure, to pass threshold of high and very higher pressures, with normal pump operation. One lumen operation can also be triggered by the operator, or by the patient on demand.”

The peritoneal dialysis system 105 can involves a portable dialysate regeneration loop (PDRL) using the recycler 110. The PDRL benefits from use of a multi-lumen catheter (as in FIG. 1, FIGS. 2A-2B, and FIGS. 4-5) in a closed circuit with the recycler 110. In some examples, the peritoneal dialysis system 105 can provide continuous PD to the patient 195 for months without having to replace or perform maintenance, with only battery charging (e.g., via induction charging by the charger 175) and/or replacing of cartridges of the substances used by the mixer 130 (e.g., substance 715, substance 725, substance 735). The closed circuit design minimizes infections, allows for portability, and removes the need for storing and shipping expensive, heavy and space occupying dialysate.

FIG. 5 is a block diagram illustrating an example of a use case 500 of a peritoneal dialysis system 105 that includes two inflow lumens 555 and two outflow lumens 550. The inflow lumens 555 and the outflow lumens 550 are divided among two contact areas, referred to as a contact area 530 and a contact area 535. The two inflow lumens 555 are labeled lumen L_{A} and lumen L_{B}. The two outflow lumens 550 are labeled lumen L_{C} and lumen L_{D}. The peritoneal cavity 190 is divided horizontally (e.g., in the direction perpendicular to the direction of gravity) into he contact area 530 and the contact area 535. The lumen L_{B} (of the inflow lumens 555) and the lumen L_{C} (of the outflow lumens 550) are arranged in, and/or coupled to, the contact area 530, with the lumen L_{C} (of the outflow lumens 550) being dependent compared to the lumen L_{B} (of the inflow lumens 555). The lumen L_{A} (of the inflow lumens 555) and the lumen L_{D} (of the outflow lumens 550) are arranged in, and/or coupled to, the contact area 535, with the lumen L_{D} (of the outflow lumens 550) being dependent compared to the lumen L_{A} (of the inflow lumens 555). The peritoneal dialysis system 105 of FIG. 5 can effectively clear bodily waste products from both the contact area 530 and the contact area 535 based on the inflow lumens 555 providing dialysate that runs down the peritoneal cavity 190, absorbs waste along the way, and becomes retrieved by the outflow lumens 550 for further recycling. In some examples, dialysate provided by the lumen L_{B} (of the inflow lumens 555) runs down at least the contact area 530, absorbs waste along the way, and becomes retrieved by the lumen L_{C} (of the outflow lumens 550) as spent dialysate. Similarly, in some examples, dialysate provided by the lumen L_{A} (of the inflow lumens 555) runs down at least the contact area 535, absorbs waste along the way, and becomes retrieved by the lumen L_{D} (of the outflow lumens 550) as spent dialysate. In this way, the FIG. 5 can effectively clear bodily waste products from different portions of the peritoneal cavity 190 in parallel.

In some examples, the peritoneal dialysis system 105 of FIG. 5 includes independent pumps for controlling driving (e.g., conveyance) of fluids through the outflow lumens 550 and inflow lumens 555. The pumps of the peritoneal dialysis system 105 of FIG. 5 include pump(s) P_{A} 505 that pushes dialysate out to the contact area 535 through the lumen L_{A} (of the inflow lumens 555), pump(s) P_{B} 510 that pushes dialysate out to the contact area 530 through the lumen L_{B} (of the inflow lumens 555), pump(s) P_{C} 515 that sucks or pulls fluid 185 (e.g., spent dialysate) from the contact area 530 of the peritoneal cavity 190 through the lumen L_{C} (of the outflow lumens 550), and pump(s) PD 520 that sucks or pulls fluid 185 (e.g., spent dialysate) from the contact area 535 of the peritoneal cavity 190 through the lumen L_{D} (of the outflow lumens 550). In some examples, independent pumps per lumen can also help with reconfiguring lumens from inflow mode to outflow mode, or vice versa. In some examples, independent pumps per lumen can also help with reconfiguring lumens from divided inflow/outflow mode to multifunctional lumen mode, or vice versa.

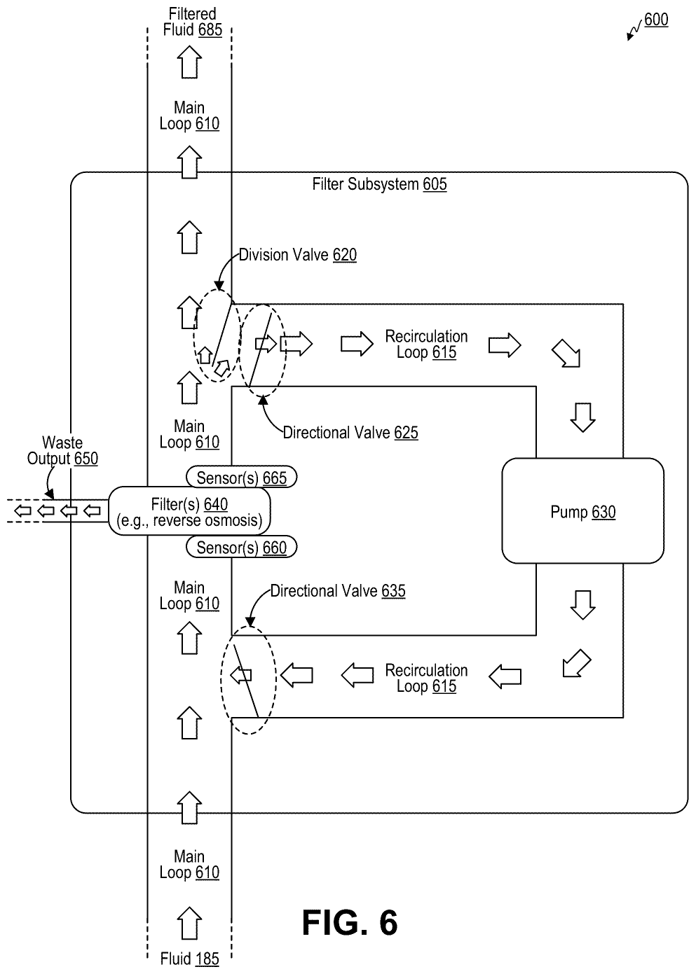

FIG. 6 is a block diagram illustrating an example of a use case 600 of a filter subsystem 605 (with a recirculation loop 615) that can be included in a peritoneal dialysis system. The filter subsystem 605 can be an example of the filter(s) 120, or vice versa. The recirculation loop 615 represents an additional loop that branches off from the main loop 610 of the recycler 110 based on the division valve 620 that divides from the main loop 610 off into he recirculation loop 615, directional valves (e.g., directional valve 625, directional valve 635), and/or a pump 640, that keep the fluid flowing clockwise in the recirculation loop 615 and eventually back to the main loop 610. The recirculation loop 615 in the filter subsystem 605 can pass the fluid through the filter(s) 640 (e.g., RO filters, carbon filters, and/or any other types of filters discussed herein) multiple times, providing improved clearance over a single pass through a filter. By adjusting the division valve 620 and/or the pump 630, the controller 160 can adjust, in real time, how much of the dialysate is being recirculated through the recirculation loop 615. In some examples, the directional valves can be omitted. In some examples, the controller can adjust the pump 630 and/or valves to direct less or more fluid through the recirculation loop 615, depending on needs to provide better clearance, depending on ultrafiltration rate, depending on availability of water, and electric power in the power supply 170. This call can be optimized for best patient outcomes, experience, and/or comfort. For example, if the battery is running low, or if the filter(s) 640 are worn out, or if the patient 195 is dehydrated, the controller 160 can turn the recirculation loop 615 off by turning off the pump 630 and/or adjusting the valves of the filter subsystem 605. If there is need to increase ultrafiltration, and/or the patient has too much fluid intake, the controller 160 can ramp up the recirculation loop 615 by adjusting the pump 630 and/or the valves of the filter subsystem 605 to provide patient with better clearance, and/or waste more fluid. In some examples, the filter subsystem 605 includes sensor(s) (e.g., sensor(s) 660 before the filter(s) 640 and/or sensor(s) 665 after the filter(s) 640)

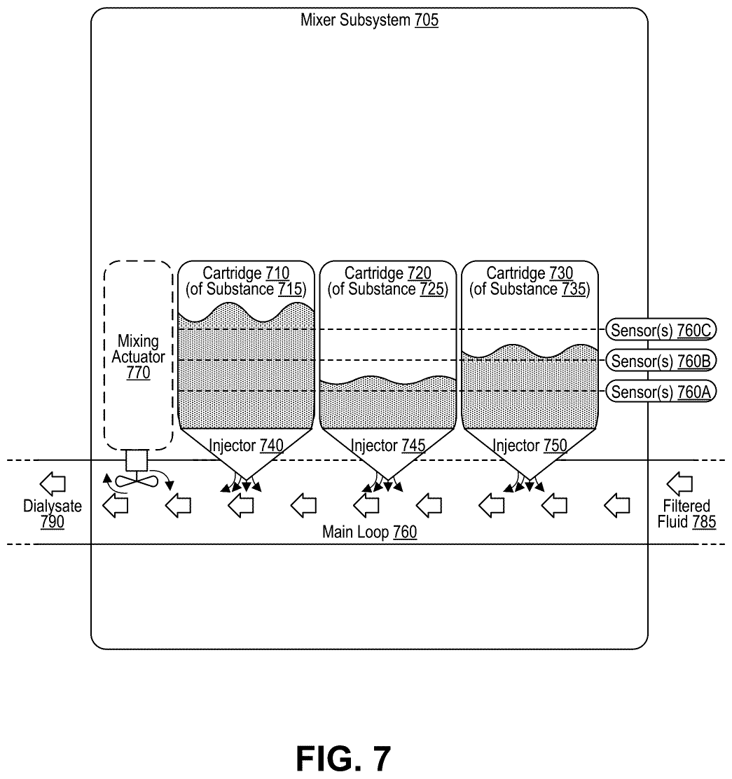

FIG. 7 is a block diagram illustrating an example of a use case 700 of a mixer subsystem 705 that can be included in a peritoneal dialysis system. The mixer subsystem 705 can be an example of the mixer 130, or vice versa. The mixer subsystem 705 receives the filtered fluid 785 (e.g., filtered fluid 685) (e.g., from the filter(s) 120 and/or the filter subsystem 605) as part of the main loop 760 of the recycler 110. As the filtered fluid 785 flows through the mixer subsystem 705 (from left to right as illustrated in FIGS. 1-5 and in FIG. 7, the mixer subsystem 705 adds predetermined portions of substances (e.g., substance 715, substance 725, substance 735) from reservoirs (e.g., cartridge 710, cartridge 720, cartridge 730) into the filtered fluid 785 using injectors (e.g., injector 740, injector 745, injector 750) to generate the dialysate 790. In particular, the substance 715 is injected from the cartridge 710 into the filtered fluid 785 using the injector 740, the substance 725 is injected from the cartridge 720 into the filtered fluid 785 using the injector 745, and the substance 735 is injected from the cartridge 730 into the filtered fluid 785 using the injector 750. In some examples, the mixer subsystem 705 may include a mixing actuator 770 that may rotate, vibrate, or otherwise introduce turbulence to help mix the substances (e.g., substance 715, substance 725, substance 735) into the filtered fluid 785 to generate the dialysate 790.

In some examples, the substances (e.g., substance 715, substance 725, substance 735) may include, for instance, water, sodium (Na⁺), potassium (K⁺), chloride (Cl⁻), bicarbonate, calcium (Ca²⁺), magnesium (Mg²⁺), phosphate, other electrolytes, glucose, dextrose, lactate, acetate, other non-electrolytes, salts (e.g., NaCl, KCl), other solutes mentioned herein, other substances mentioned herein, or a combination thereof. In some examples, these solutes may be, or may include, ions and/or anions, such as sodium ions (Na⁺), chloride anions (Cl⁻), calcium ions (Ca²⁺), magnesium ions (Mg²⁺), other ions, other anions, or a combination thereof. In some examples, these substances (e.g., substance 715, substance 725, substance 735) may be, or may include, molecules, such as bicarbonate, phosphate, glucose, dextrose, lactate, acetate, salts, water, other molecules, or a combination thereof. In some examples, these substances (e.g., substance 715, substance 725, substance 735) may be, or may include, fresh water, for instance to help water down the filtered fluid 785 further before adding the solutes (e.g., if the filtered fluid still retains more than a maximum threshold amount of an undesirable solute such as urea or another waste product).

In some examples, the mixer subsystem 705 may include sensors (e.g., sensors 760A-760C) that identify how much of each substance is in each reservoir. For instance, the sensors 760A-760C illustrated in FIG. 7 each correspond to different threshold amounts of each substance, with the sensor 760A monitoring whether there is at least a first (low) threshold of a given substance in a given reservoir, the sensor 760B monitoring whether there is at least a second (medium) threshold of the given substance in the given reservoir, and the sensor 760C monitoring whether there is at least a third (high) threshold of the given substance in the given reservoir. Sensor data from the sensors 760A-760C can be monitored to identify how much of each substance remains in each reservoir, for instance to alert the patient (or another user) to make an adjustment to refill a specific reservoir when that reservoir is low (e.g., at or below the first threshold or second threshold). In the example illustrated in FIG. 7, the cartridge 710 includes more than the third (high) threshold of the substance 715, the cartridge 720 includes more than the first (low) threshold but less than the second (medium) threshold of the substance 725, and the cartridge 730 includes more than second (medium) threshold but less than the third (high) threshold of the substance 735. In some examples, based on the readings from the sensors 760A-760C, the peritoneal dialysis system 105 can alert the patient (or another user) (e.g. via a user device 1350) to refill the cartridge 720 with more of the substance 725. In some examples, to reduce risk of infection, the entire mixer can be replaced, with all cartridges permanently hosted within it.

While the mixer subsystem 705 is illustrated with three reservoirs of three substances, it should be understood that the mixer subsystem 705 can add more than three substances to the filtered fluid 785 to generate the dialysate 790. Likewise, it should be understood that the mixer subsystem 705 can add less than three substances to the filtered fluid 785 to generate the dialysate 790. In some examples, the mixer subsystem 705 can add predetermined (or dynamic) amounts of each of the substances to the filtered fluid 785 to generate the dialysate 790. These amounts can be different for each substance. In an illustrative example, the mixer subsystem 705 can add, to the filtered fluid 785, 132-145 milliEquivalents/Liter (mEq/L) of sodium, 2.0-5.0 mEq/L of potassium, 90-108 millimoles/Liter (mmol/L) of chloride, 18-30 mmol/L of bicarbonate (e.g., carbon dioxide (CO₂)), 2.00-2.55 mmol/L of calcium, 0.50-0.95 mmol/L of magnesium, 0.5-1.3 mmol/L of phosphate, or a combination thereof. In some examples, the predetermined amounts of each of the substances to be added to the filtered fluid 785 by the mixer subsystem 705 to generate the dialysate 790 can be based on reference ranges of electrolytes in a healthy patient. In some examples, the predetermined amounts of each of the substances to be added to the filtered fluid 785 by the mixer subsystem 705 to generate the dialysate 790 can be lower than reference ranges of electrolytes in a healthy patient by amount(s) of the substances that usually remain in the filtered fluid 785. For instance, some sodium can remain in the filtered fluid 785 after filtering, meaning that the mixer subsystem 705 does not need to add the full reference range of sodium. In some examples, the predetermined amounts of each of the substances to be added to the filtered fluid 785 by the mixer subsystem 705 to generate the dialysate 790 can be lower than reference ranges of electrolytes in a healthy patient for substances that are desirable to reduce in dialysis patients, such as potassium.

As noted previously, in some cases, one of the substances (e.g., substance 715, substance 725, substance 735) may be, or may include, water. For instance, in some examples, the filters (e.g., filter(s) 120, filter subsystem 605, filter(s) 640, reverse osmosis filter(s)) may have limited efficiency. Some substances, such as certain waste products (e.g., urea) may not be cleared efficiently enough in some circumstances. To reduce the concentration of such substances in the dialysate 790, the mixer subsystem 705 may add water to the filtered fluid 785 as part of generating the dialysate 790, to water down the concentration of such substances in the dialysate 790. In some examples, the water (and/or other substances) may be injected by the mixer subsystem 705 after the filter (e.g., filter(s) 120, filter subsystem 605, filter(s) 640) as illustrated in the main loop of the recycler 110 of FIGS. 1-5. In some examples, the water and/or some of the other substances may be injected by the mixer subsystem 705 before the filter (e.g., filter(s) 120, filter subsystem 605, filter(s) 640) in the main loop of the recycler 110, or even or even as part of the filter subsystem 605 (e.g., in the recirculation loop 615 of the filter subsystem 605). In some examples, Adding and/or injecting even one or two liters of water per 24 hours can improve the effective clearance (e.g., reduction of urea, or other substance from between spent dialysate vs. in the fresh dialysate line) very significantly, providing better overall clearance to patients. Adding water and/or substances to the mixer subsystem 705 is less work, is less time-consuming, takes less space, and provides less infection risk than exchanging large volumes of dialysate (e.g., 12 L) every day, 3-4 times a day, thus improving over traditional peritoneal dialysis systems.

In some examples, the substances injected by the mixer subsystem 705 may include initial or complete therapies, empirically, including antibiotics such as gentamicin, vancomycin, other medications, and the like. In some examples, the mixer subsystem 705 can inject such substances before a final diagnosis is done, but after sample of the fluid is stored in part of waste container, before an evaporator, so that the antibiotics do not affect the results of culture.

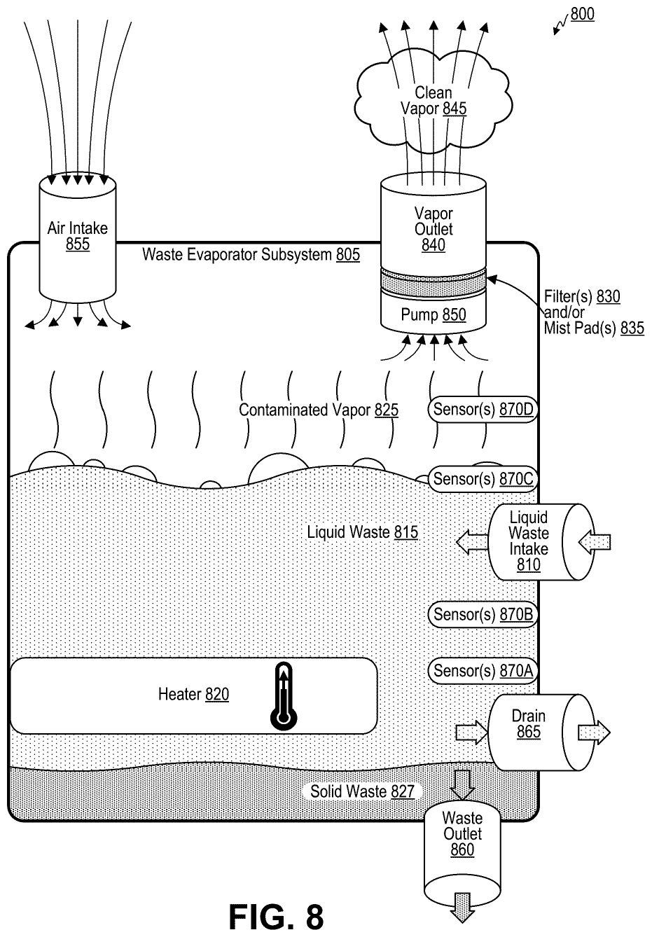

FIG. 8 is a block diagram illustrating an example of a use case 800 of a waste evaporator subsystem 805 that can be included in a peritoneal dialysis system. The waste evaporator subsystem 805 is an example of the waste container 125 with an evaporator, or vice versa. The waste evaporator subsystem 805 receives liquid waste 815 from the filter(s) (e.g., from the filter(s) 120, the filter subsystem 605, the filter(s) 640, and/or the waste output 650) via the liquid waste intake 810. A heater 820 (powered by the power supply 170) heats the liquid waste 815 to evaporate some of the liquid from the liquid waste 815 into contaminated vapor 825. As a result of the evaporation, some of the liquid waste 815 can condense (in volume) down into solid waste 827, which can fall to the bottom of the waste evaporator subsystem 805 to be eventually evacuated via the waste outlet 860 and/or the drain 865. The drain 865 can also be used to drain the liquid waste 815. The contaminated vapor 825 can be suctioned using negative pressure by a pump 850, passed through filter(s) 830 and/or mist pad(s) 835 to clean out contaminants from the contaminated vapor 825, and output as clean vapor 845 via the vapor outlet 840. The filter(s) 830 and/or mist pad(s) 835 and clean out the contaminants to reduce odors and/or toxicity from the contaminated vapor 825. In some examples, the waste evaporator subsystem 805 includes an air intake 855 that receives clean air from the environment to help move the contaminated vapor 825 toward the pump 850, the filter(s) 830, the mist pad(s) 835, and/or the vapor outlet 840.

In some examples, the waste evaporator subsystem 805 also includes sensors 870A-870D that can identify how much waste (e.g., liquid waste 815 and/or solid waste 827) is in the waste evaporator subsystem 805. The sensors 870A-870D are positioned at different levels within the waste evaporator subsystem 805.

In some examples the entire evaporator can be replaced from time to time, and it would not need then need a drain.

In some examples, there is a container for waste. In some examples, the waste evaporator subsystem 805 includes a one way valve and/or is replaceable.

In some examples, the waste container 125 and/or waste evaporator subsystem 805 includes a port for culture collection within waste. For instance, a waste compartment, before evaporation is used, can use a needle and/or culture syringe to collect cultures. The cultures can be collected in a detachable culture bottle, specimen bottle, or cartridge. The culture bottle, specimen bottle, or cartridge can be detached without breaking the closed. The culture can be analyzed using sensor(s) (e.g., onboard the peritoneal dialysis system 105 or external to the peritoneal dialysis system 105), with the sensor data from the sensor(s), and/or any other determinations or predictions based on the sensor(s) (e.g., symptoms or diagnoses determined or predicted based on characteristics of the cultures), based on the being considered as context data 1410 in some examples.

The waste evaporator subsystem 805 represents an improvement over a waste container 125 without an evaporator by reducing the volume of waste, and reducing or removing odor from the resulting steam. In some examples, the waste evaporator subsystem 805 can completely evaporate the liquid from the liquid waste 815, leaving only solid waste 827. In some examples, this can provide patients with more options for disposing of the waste. For instance, while liquid waste 815 might need to be disposed of in a toilet, solid waste 827 can, in some cases (e.g., depending on local laws) be disposed of in additional ways (in addition to a toilet), such as a biohazardous waste bin, a regular trash bin, or even a compost bin. The waste evaporator subsystem 805 would allow patients to need draining of the fluid (no need to go back home every 3-7 hours to drain the fluid), reducing chances of infection, reducing manipulation around the catheter, and reducing patient stress with planning.

Creation of traditional dialysate for traditional PD (de novo) is a slow and complicated process, involving purifying water by cleaning tap water from: bacteria (e.g., with resins, carbon, microfiltration, multimedia filter), endotoxins, chloramine, Cl (chlorine) (e.g., with activated carbon filters), heavy metals, (e.g., via desalinization), other electrolytes (e.g., K, Na, Cl, Mg), and adding specified substances (e.g., salts, glucose, electrolytes) to the filtered water.

However, unlike the traditional dialysate making process that uses municipal water to crate dialysate, the recycler 110 of the peritoneal dialysis system 105 makes clean dialysate from spent dialysate. In most cases spent dialysate does not have bacteria, chloramine, Cl (chlorine), endotoxins, heavy metals, or certain electrolytes, meaning that certain filters (e.g., carbon filter, resin filter, microfiltration, multimedia filter, desalinization) may be omitted from the filter(s) 120 and/or filter subsystem 605 in some examples of the peritoneal dialysis system 105, improving portability and battery life.

In some examples, spent dialysate may include cellular debris, proteins, organic and electrolytes. These can be filtered by carbon filter, multimedia filter and/or reverse osmosis filters. These filters can be replaced with the system, in a sterile way, periodically. Even so, the peritoneal dialysis system 105 can continue to operate in a close circuit even as individual components are replaced, in some cases pausing or rerouting flow temporarily. In some examples, the tubing and/or lines (e.g., catheter 165, catheter 1320, catheter 1340, and/or lumens) can have connectors that can be connected to and disconnected from the patient 195. They are connecting to the tubes. In some examples, the peritoneal dialysis system 105 can include ultraviolet (UV) light sources that are configured to shine on connectors and/or tubes to prevent infection in case the connectors and/or tubes need to be disconnected and/or reconnected.

In most examples, organic waste products (e.g., urea and/or peptides) do not need to be cleared out all in one pass. In continuous dialysis, rejecting even 5% of undesired substances (and for most electrolytes and other substances with molecular weight above 150 rejection is closer to 95%) with each pass of filtering (e.g., via the filter(s) 120 and/or filter subsystem 605), assuming the filtering runs continuously (e.g., for 24 hours), results in satisfactory total daily clearance and health for a patient 195.

In some examples, electrolytes (e.g., potassium, sodium, chloride) are rejected bu the filter(s) 120 and/or the filter subsystem 605 to waste with at least 90% rejection rates. In some examples, the peritoneal dialysis system 105 can include serial filtering (e.g., RO filtering) with some re-circulation (e.g., via the recirculation loop 615) or without re-circulation of concentrate after the first or second reverse osmosis membrane, to provide closer to 100% rejection rates (“two-pass RO”). This can improve filtering, but can in some cases increase energy consumption and/or complexity.

Calculations indicate that, the filter(s) 120 having a rejection rate of 90-100% for sodium, the mixer 130 can safely add predetermined amounts of electrolytes blindly to the filtered fluid 785 (without having to measure the concentration of those electrolytes in the filtered fluid 785 first) to generate dialysate 790. These calculations are laid out in table 900, table 1000, and table 1100 of FIGS. 9-11.

FIG. 9 is a table 900 illustrating changes to sodium level before and after use of a dialysate that is sodium-based in a patient who has a high initial level of sodium (170) and with a filter that has a low rejection rate of sodium (0.90).

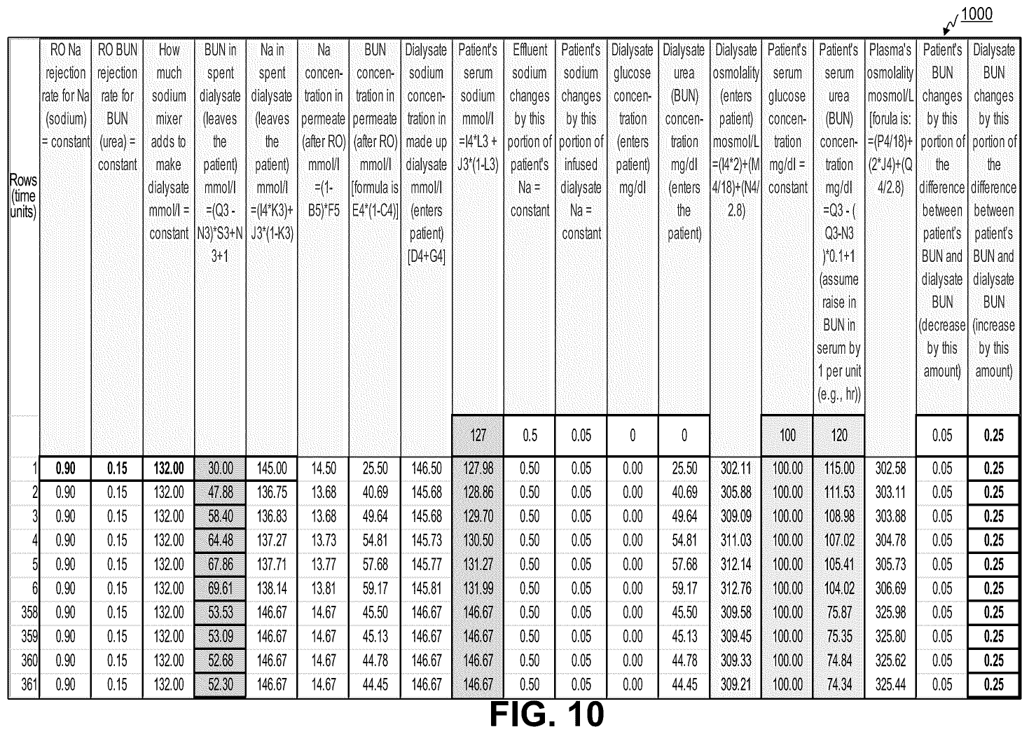

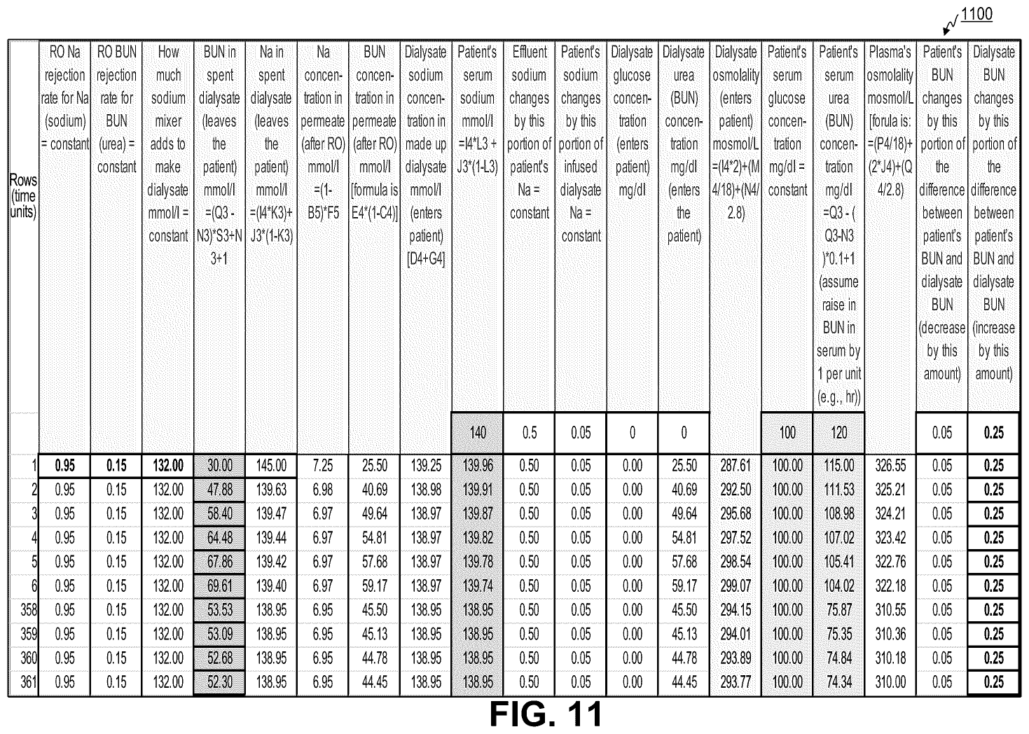

Table 900 of FIG. 9, table 1000 of FIG. 10, and table 1100 of FIG. 11 all have the same columns. They illustrate the clearance of estimations for sodium. Sodium has one of the highest concentrations (in mmols/l) of all electrolytes, so it is subject to highest absolute differences in concentration in the recycled/regenerated dialysate. It be even easier to maintain stable and predictable concentrations for all other substances without measuring their concentration. Consider: there is almost no difference for body whether the potassium is 4 or 4.4 (10% relative difference) but there is much more difference for the body whether sodium is 150 or 165 (10% difference).

Table 900, illustrates a case where patient's initial sodium is 170, which is very high.

The columns are named appropriately. Some of the formulas are simplified to show the methods and systems presented herein. Each row represents 1 unit of time (e.g., 1 hour). The dialysis gradually decreases the sodium level to 146.6 (almost normal) after 360 units of time. Very slowly, and safely, without knowing what the concentrations of electrolytes in dialysate are—which simplifies all. The osmolality of the serum and even without glucose, slowly but surely decreases. The urea of patient lowers, achieve some lower than initial (120) level (74), which means patient is receiving effective dialysis. Even with a poor rejection rate of 0.15 (which is very pessimistic) for urea, the dialysis is still working to clean waste from the patient. The peritoneal dialysis system 105 clears other waste products, like phosphorus, sodium, chloride, potassium, beta2—microglobulin or macroglobulin, or other proteins. The peritoneal dialysis system 105 performs this clearance of waste products without having to know the concentration of electrolytes in the filtered fluid 785, in some examples. Note that table 900 has its 5^{th} through 360^{th} rows hidden to preserve space on the table and allow a broader range of data to be shown.

In some examples the concentrations of various electrolytes and substances are measured (e.g., via sensor(s)) and known to the peritoneal dialysis system 105, and the peritoneal dialysis system 105 can provide even more tailored and personalized dialysis to the patient as a result.

In some examples, RO membranes have rejection rates for urea (and/or other non-charged small organic molecules) of about 0.15-0.33. In some examples, RO membranes are more efficient in rejecting urea and other such substances—the table 900 illustrating a conservative or pessimistic estimate for a patient who constantly generates urea at a rate that would raise the serum concentration of urea by 1 every unit of time if there was no clearance at all (e.g., patient with zero native kidney function and relatively slow flow and regeneration of dialysate: about 15 ml/min).

There are ways to improve the clearance of urea (and other small organic molecules), for instance including more dialysate flow, inserting various chemicals (e.g., changing the pH of the spent dialysis when urea is removed) temporarily, sending electric current via spent dialysate as it is passing through the RO membrane, shining UV light and/or radiation, or a combination thereof. Such operations can increase clearance of the urea (and/or other small organic molecules).

In the first unit of time (illustrated between the first and second rows) if the mixer 130 adds 132 mmol/L of sodium, blindly, without knowing that the sodium in permeate is, the newly made dialysate have sodium concentration of 132+17 mmol=149 mmol/l. Thus, the newly made dialysate have lower sodium concentration than the sodium concentration in the patient's body (which starts at 170 in the table 900). Gradually, due to fluid shifts, osmosis and thirst, the peritoneal dialysis system 105 reduces patient's sodium level and the peritoneal dialysis system 105 leads to Na of both dialysate and patient approach asymptote.

In some examples, it is safe to infuse dialysate with sodium concentration of 149 mmol/l (or initially 132 mmol/l, although the CFPD would be starting super slowly and gradually increase the rate) to a PC of a patient, who has serum sodium concentration of 170 mmol/l. For instance, it is safer to gradually infuse dialysate with sodium concentration of 149 mmol/l (132 mmol/l when running slower flows) with the CFPD at the rate of starting at 1 cc/min and gradually increasing it to 15-20 cc/min, than to infuse 2-3 L of traditional PD dialysate (within 20 minutes) with sodium concentration of 132 mmol/l, and to have the traditional PD dialysate dwell in peritoneum for 2-3 hours, as in traditional PD. This case illustrated in the table 900 is an extreme case, however. The patient in the table 900, having a sodium level of 170 mmol/l, would likely be in the intensive care unit (ICU), and would be dialyzed using more intensive and invasive procedures than even traditional PD.

On the other hand, 99% of patients receiving PD have serum sodium concentration between 128-156. For these patients, the peritoneal dialysis system 105 is very safe.

Other electrolytes, like K, Cl, have lower concentration that sodium (Na) so rejection of 90-100% or more (as delivered for these electrolytes via the filter(s) 120) is sufficient. With 90% rejection, interestingly, due to internal characteristics of the recycler 110, the sodium from spent dialysate is not fully removed by the filter(s) 120, allowing for mildly higher osmolality (vs. other sodium-based dialysates with sodium level of 132). This may remove any need to add any glucose, or at least minimize the amount of needed glucose to be added to dialysate. This would further decrease the need for diabetic medications, infectious complications, incident insulin resistance, obesity, and/or diabetes in CFPD patients using the peritoneal dialysis system 105.

One way to know how much glucose to add, if any, is to measure conductivity (for which sodium is the main driver) of the permeate (e.g., via the lumen sensor(s) and/or the sensor(s) of the sorter(s)). If the conductivity is high, the mixer 130 can add a predetermined (or dynamic) amount of glucose in that scenario, so the dialysate osmolality is mildly higher than patients' osmolality to allow for UF.

With CFPD using the peritoneal dialysis system 105, patients do not need to prepare traditional dialysate (e.g., 12 L of traditional dialysate) before every night, like is done for patients receiving traditional PD as they sleep (e.g., enough for 10 hours). The peritoneal dialysis system 105 can provide CFPD for 24 hours or more without interruption, so the peritoneal dialysis system 105 can provide effective PD on a more gradual timescale than traditional PD, as the peritoneal dialysis system 105 has more time to provide PD than a traditional PD. This more gradual timescale can also reduce trauma to the PM, helping the PM retain permeability for longer.

FIG. 10 is a table 1000 illustrating changes to sodium level before and after use of a dialysate that is sodium-based in a patient who has a low initial level of sodium (127) and with a filter that has a low rejection rate of sodium (0.90).

Table 1000 illustrates a case where patient's initial sodium is 127, which is very low. The dialysis gradually increases the sodium level to 146.6 (almost normal) after 360 units of time, gradually, and safely, without the peritoneal dialysis system 105 having to check what the concentrations of electrolytes in dialysate are, simplifying the operation of the peritoneal dialysis system 105.

The osmolality of the serum, even without glucose, slowly but surely decreases. The urea of patient lowers, achieving a lower level (74) than the initial level (120), which means the patient is receiving effective dialysis. Even with a filter with a poor rejection rate of 0.15 for urea, the peritoneal dialysis system 105 still works well. The peritoneal dialysis system 105 clears all other waste products, like phosphorus, sodium, chloride, potassium, all without having to know the concentration of electrolytes, and using the same equipment (e.g., the peritoneal dialysis system 105) as for table 900. The peritoneal dialysis system 105 clears other waste products, like beta2—microglobulin or macroglobulin, or other proteins.

In some examples the concentrations of various electrolytes and substances is detected via sensors and known to the peritoneal dialysis system 105, and even more tailored and personalized dialysis can be provided by the peritoneal dialysis system 105 based on these concentrations. Note that table 1000 has its 5th through 360th rows hidden to preserve space on the table and allow a broader range of data to be shown.

In some examples, RO membranes have rejection rates for urea (and/or other non-charged small organic molecules) of about 0.15-0.33. In some examples, RO membranes are more efficient in rejecting urea and other such substances—the table 1000 illustrating a conservative or pessimistic estimate for a patient who constantly generates urea at a rate that would raise the serum concentration of urea by 1 every unit of time if there was no clearance at all (e.g., patient with zero native kidney function and relatively slow flow and regeneration of dialysate: about 15 ml/min).

FIG. 11 is a table 1100 illustrating changes to sodium level before and after use of a dialysate that is sodium-based in a patient who has a low initial level of sodium (140) and with a filter that has a high rejection rate of sodium (0.95).

Table 1100 illustrates a case where patient's initial sodium is 140, which is very low. The columns are named appropriately. In table 1100, the dialysate is run at the same pace (flow rate of dialysate) as in Table 900. For instance, the patient now generate 1 unit of the BUN for every unit of time, as in the Table 900. Each row represents 1 unit of time, which in some examples can be 1 hour. The dialysis starts with patient's sodium level of 140 (normal) before it goes down to 139 (still normal) in next 360 units of time. This is a less pessimistic scenario (with assumed rejection rate of sodium at 0.95, which most of RO membranes can easily provide). The peritoneal dialysis system 105 is able to perform this dialysis gradually, and safely, without having to know what the concentrations of electrolytes in dialysate are, which allows the peritoneal dialysis system 105 to be portable and provide a simplified patient experience. Even with a poor rejection rate of 0.15 for urea, the peritoneal dialysis system 105 still performs effective dialysis. The peritoneal dialysis system 105 clears out all other waste products, like phosphorus, sodium, chloride, potassium, other waste products, like beta2—microglobulin or macroglobulin, or other proteins, again without having to know the concentration of electrolytes, and using the same equipment as in table 900 (e.g., the peritoneal dialysis system 105). Note that table 1100 has its 5th through 360th rows hidden to preserve space on the table and allow a broader range of data to be shown.

In some examples the concentrations of various electrolytes and substances is detected via sensors and known to the peritoneal dialysis system 105, and even more tailored and personalized dialysis can be provided by the peritoneal dialysis system 105 based on these concentrations. In some examples, RO membranes have rejection rates for urea (and/or other non-charged small organic molecules) of about 0.15-0.33. In some examples, RO membranes are more efficient in rejecting urea and other such substances—the table 1100 illustrating a conservative or pessimistic estimate for a patient who constantly generates enough urea at a rate that would raise the serum concentration of urea by 1 every unit of time if there was no clearance at all (e.g., patient with zero native kidney function and relatively slow flow and regeneration of dialysate: about 15 ml/min).

Regarding the mixer of the peritoneal dialysis system 105 (e.g., the mixer 130 and/or the mixer subsystem 705), the mixer can add to permeate (mostly water) sterile high concentrations mixtures from cartridges (at the given amounts and ratios of amounts to get desired concentration) of electrolytes that are in all dialysate like: sodium (Na⁺), potassium (K⁺), calcium (Ca²⁺), magnesium (Mg²⁺), chloride (Cl−), and bicarbonate and add glucose or dextrose at desired concentrations.

In some examples, it is feasible, as shown for sodium above, to add stable, constant concentration of all electrolytes and/or substances to provide effective PD via the peritoneal dialysis system 105. In some examples, the mixer can provide an adjustable amount of glucose and/or sodium to adjust a target UF. In some examples, the peritoneal dialysis system 105 can allow a patient or other user to adjust UF, or can adjust UF automatically (e.g., by adjusting the amount of sodium and or glucose), depending on blood pressure, symptoms, blood pressure, weight, fluid intake, urine production. UF can also be automatically adjusted using impedance, weight, and other edema sensor (stretchable band on shin, sensing the amount of stretch) and the controller 160 and/or machine learning system 1400.

In some cases, clearance of small sized (up to 150 Daltons) and uncharged substances (like urea) can be tackled in two ways. In some examples, due the nature of the peritoneal dialysis system 105, and the fact that dialysate is recirculated, and because the mixer does not add any of these substances into the permeation, and that organic uncharged substances can be gradually removed from PC, even a 0.1 (10%) rejection rate can provide satisfactory of such substances over time, since with each pass-through filtering (e.g., with a RO filter of the filter(s) 120), 0.1 (10%) of urea and substances alike be removed. The peritoneal dialysis system 105 only needs to remove these substances from the peritoneal cavity 190 to the waste container 125 faster than they are made in body, which the peritoneal dialysis system 105 can achieve. The peritoneal dialysis system 105 can maintain a stable, balanced concentration of these substances in the peritoneal cavity 190 that is lower than a toxic concentration. For example, patients with chronic kidney disease and with chronic urea serum concentration of 50 mg/dl or even 70 mg/dl are usually completely fine, and patients get “uremic” (sick from accumulation low molecule uncharged organic substances like urea) when their urea level reaches 90-120 mg/dl or so. Because the peritoneal dialysis system 105 can maintain levels of urea below 90 mg/dl, the peritoneal dialysis system 105 maintains patients at a good and stable level of health. In some examples, RO membranes that allow much higher rejection rates up to 99.9% (0.999) for some of these substances can be used.

The peritoneal dialysis system 105 can also remove almost all unwanted proteins from the peritoneal cavity 190, as proteins swiftly get rejected by RO membranes (e.g., Beta 2 micro-globulin) of the filter(s) 120. Traditional dialysis system struggle to remove adequate amounts of immunoglobulin, or Beta 2 micro-globulin. In fact, described herein CFPD using the peritoneal dialysis system 105 most likely does a better job removing beta-2 macroglobulin (and other undesirables proteins and similar substances) than traditional PD, allowing for better long-term outcomes for patients.

In some examples, the peritoneal dialysis system 105 can operate at 10-25 cc/min for 16-24 hours a day of total dialysate flow to match or deliver more dialysis than what the traditional PD modalities provide. At the same time, the peritoneal dialysis system 105 provides a number of other benefits that traditional peritoneal dialysis systems do not provide, such as CFPD, portability of the peritoneal dialysis system 105, no need for a (large, heavy, expensive) reservoir of fresh or used dialysate (since the flow is continuous and the recycler 110 generates new dialysate), closed circuit (reducing risk of infection and improving safety), charging through induction (no risk of infections), largely automatic operation with minimal or no need for input for the patient or other operators, no need to limit fluid intake (which is a problem for traditional PD, and actually increased water intake may be desired to supplement more water loss which is rejected by RO, and having more PO intake, and being able to reject more water by RO would allow for better clearance associated with more flow through RO) less risk of diabetes (e.g., due to reliance on sodium rather than glucose for the osmotic gradient), less risk of infectious complications, less need for replacement of components (e.g., only need to replace cartridges after multiple days, weeks, or months), less expensive over time than traditional dialysis, and other improvements and benefits discussed herein.

In some examples, there is rejected water (with rejected waste products) (e.g., fluid 185) going into the waste based on operation of the filter(s) 120 and/or the filter subsystem 605. To replace this water, in some examples, patients may need to drink an amount of water equivalent to about 15-20% of dialysate used (as this is waste). So, for 15 cc/min of dialysate flow, there may be about 15*60*24*0.2=about 2.8 L of daily waste. This is how much water a patient should drink to offset the system wasting this much water daily (mostly water rejected by RO). This would provide 14400 cc or 14.4 L of dialysate, which would provide adequate clearance.

The sensors discussed herein can be used to detect infection (e.g., based on changes in temperature, pH, and/or conductivity) by the controller 160 and/or machine learning system 1400. The controller 160 and/or machine learning system 1400 can detecting infections by comparing current sensor data (or recent sensor data) from sensors with historical sensor data for the patient, and/or to other patients' data recorded from similar sensors of other peritoneal dialysis systems performing peritoneal dialysis in other patients.

In some examples, the hydrostatic pressure sensor of lumen with most dependent tip (MDL) can be used to maintain appropriate residual peritoneal dialysate (RPD). During a process called RPD determination and set up, the peritoneal dialysis system 105 can stop outflow via MDL(s), and only allow inflow via NMDL(s). The peritoneal dialysis system 105 can receive pressure measured by hydrostatic pressure sensor(s) associated with the NMDL. This pressure may be presented to the controller 160, the machine learning system 1400, and/or an operator (e.g., physician or RN). The operator, based on patient's body habitus (PC volume when height of fluid in PC is known depends mostly on the cross-sectional area of the PC in the plane parallel to the ground, or sea) be able to set desired initial RPD with, based on hydrostatic pressure, which is (not linearly, but still) proportional to RPD.

Additionally, as the infusion of dialysate via NMDL(s) continues, each infused volume correlates to (hydrostatic) pressure measured at the sensor connected to MDL, these both are registered in the memory of the controller 160. The controller 160 can later, based on recorded correlations, establish a current (or recent) volume of dialysate in the peritoneal cavity 190, based on hydrostatic pressure measured by the sensor.

The system let the fluid 185 out of peritoneal cavity 190, via MDL (e.g., outflow lumen(s)), when hydrostatic pressure reaches at least the pressure correlating with desired by operator RPD. This is possible because earlier, each RPD amount (with inflows opened and closed outflow lumens) was correlated with various hydrostatic pressures registered by hydrostatic pressure sensor of the MDL.

In some examples, the optimal RPD for the peritoneal dialysis system 105 is the highest RPD where the patient is still comfortable (having more dialysate in the peritoneal cavity 190 is less comfortable). Generally, the higher the RPD, the more saturation of dialysate happens, and more efficient dialysis is.

If the patient or operator desires further optimization of RPD for patient's comfort and/or maximal efficiency dialysis, RPD may be found during the during the maintenance of the invention, for instance during the patient's visits with a provider. To find and/or perform optimal RPD, an operator can gradually increase RPD until KT/V does not improve by more than delta KT/V (e.g., where delta KT/V is approximately 1-10%) and compare RPDs with historical data from the patient and/or or other patients, for instance using the controller 160 and/or the machine learning system 1400.

In some examples, the peritoneal dialysis system 105 can adjust the temperature of in-flowing dialysate to allow for better mixing of newly in-flowing dialysate (e.g., by the mixer 130 and/or the mixer subsystem 705) and RPD. For example, infusing dialysate at a lower temperature than one of RPD probably make that new portion “go down” (as being heavier) and mix better with dialysate in the peritoneal cavity 190, based on convection.

In some examples, a patient, operator, or the peritoneal dialysis system 105 itself may adjust the desired UF by adjusting various sodium or glucose concentrations in the dialysate (e.g., added via the mixer 130 and/or mixer subsystem 705) and/or by adjusting or applied pressure on the filter(s) 120 (e.g., on the RO membranes). Such adjustments can be determined by the controller 160 and/or the machine learning system 1400, for instance, based on the patient's symptoms, history, impedance, conductivity, measured cardiac output, weight changes, blood pressure, heart rate, using past historic data of patients or other patients, and/or other context data 1410. The peritoneal dialysis system 105 can automatically adjust glucose, sodium, rejection rates of RO complex to adjust osmolality of dialysate (e.g. as adjustment(s) 1435), which leads to adjustment of UF.

In some embodiments, if a patient 195 feels they need more fluid removed (e.g., more UF), the patient 195 can boost UF by providing an input (e.g., pressing a button) (e.g., on the peritoneal dialysis system 105 or the user device 1350) to increase glucose, or sodium, or other osmotically active substances, in the inflow dialysate to increase UF. In some examples, the mixer 130 can add other substances to the dialysate, such as medications (e.g., drugs, antibiotics), for instance to automatically treat an infection based on and in response to detection of signs of infection. For instance, a patient 195 can request more fluid removed (e.g., more UF) when the patient knows they are heavier than usual, drinking more water, and/or having a larger salt intake.

FIG. 12 is a graph diagram 1200 illustrating changes to volume and flow over time based on use of a dialysis system. To provide better mixing of newly inflow dialysate with RPD (“residual volume” on FIG. 2) various inflow patterns may be used. For instance, the graph diagram 1200 includes a volume graph 1205 and a flow graph 1210, both with horizontal time axes 1215 with marks at times t_{A}, t_{B}, t_{C}, and t_{D}. The flow graph shows inflow 1250 in solid lines, with periodic increases from flow value f_{A} to flow value f_{B} (along a vertical flow axis 1225) when the residual volume 1230 (along the vertical volume axis 1220) is rising (toward volume v₂) and decreases from flow value f_{B} to flow value f_{A} to when the residual volume 1230 is falling (toward and below volume v₁). The flow graph shows outflow 1255 in dashed lines, with periodic increases in the absolute value of the negative flow (decreases in the value of the flow) from flow value f_{D} to flow value f_{E} when the residual volume 1230 (along the vertical volume axis 1220) is rising (toward volume v₂) and decreases in the absolute value of the negative flow (increases in the value of the flow) from flow value f_{E} to flow value f_{D} to when the residual volume 1230 is falling (toward and below volume v₁). In some embodiments RPD will go to zero and in some it will stay stable, and the inflows and outflows will adjust accordingly to maintain any desired RPD.

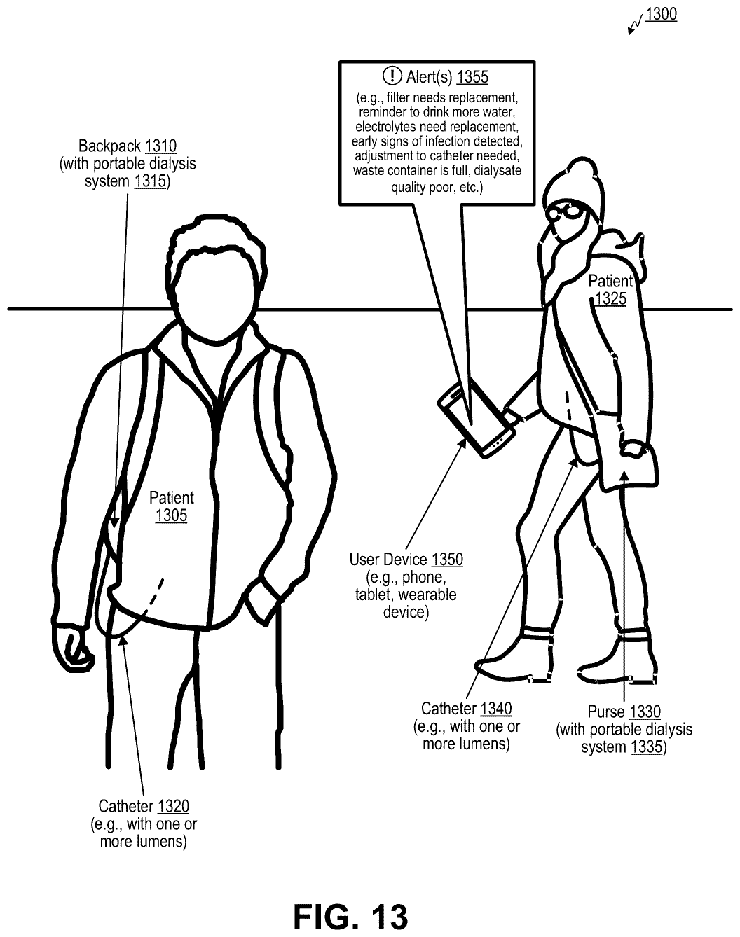

FIG. 13 is a conceptual diagram illustrating a scene with a patient 1305 wearing a backpack 1310 with a portable dialysis system 1315 and a patient 1325 wearing a purse 1330 with a portable dialysis system 1335. The portable dialysis system 1315 and/or the portable dialysis system 1335 can be examples of the peritoneal dialysis system 105 of any of FIGS. 1-5, or vice versa. A catheter 1320 (e.g., with one or more lumens) runs from the portable dialysis system 1315 in the backpack 1310 to the peritoneal cavity (e.g., peritoneal cavity 190) of the patient 1305. In some examples, inside the peritoneal cavity (e.g., peritoneal cavity 190) of the patient 1305, the lumens of the catheter 1320 can branch out in different directions so that at least one lumen is dependent relative to the other lumen(s), with the dependent lumen(s) being designated (e.g., by the controller 160) as the outflow lumen (e.g., outflow lumen 150) and the non-dependent lumen(s) being designated (e.g., by the controller 160) as the inflow lumen (e.g., inflow lumen 155).

A catheter 1340 (e.g., with one or more lumens) runs from the portable dialysis system 1335 in the purse 1330 to the peritoneal cavity (e.g., peritoneal cavity 190) of the patient 1325. In some examples, inside the peritoneal cavity (e.g., peritoneal cavity 190) of the patient 1325, the lumens of the catheter 1340 can branch out in different directions so that at least one lumen is dependent relative to the other lumen(s), with the dependent lumen(s) being designated (e.g., by the controller 160) as the outflow lumen (e.g., outflow lumen 150) and the non-dependent lumen(s) being designated (e.g., by the controller 160) as the inflow lumen (e.g., inflow lumen 155).

The patient 1325 is also illustrated holding a user device 1350. The user device 1350 is illustrated as a phone or other mobile handset, but can be a phone, a mobile handset, a tablet, a laptop, a watch, a headset, a head-mounted device (HMD), an augmented reality (AR) device, an virtual reality (VR) device, a mixed reality (MR) device, an extended reality (XR) device, a vehicle computer, a video game console, a music player, a movie player, a television, a monitor, a speaker, a pair of headphones, a computing system 1600, any other type of computer or device discussed herein, or a combination thereof. In some examples, the portable dialysis system 1335 can send communication(s) to the user device 1350 via respective communication interfaces (e.g., communications interface 1640) of the portable dialysis system 1335 and the user device 1350, for instance wirelessly or over one or more wires (that couple the portable dialysis system 1335 to the user device 1350). The sending of the communication(s) from the portable dialysis system 1335 to the user device 1350 can cause the user device 1350 to output one or more alert(s) 1355, for instance displaying the alert(s) 1355 via a display of the user device 1350, playing the alert(s) 1355 via a speaker of the user device 1350, actuating a haptic feedback actuator to vibrate the user device 1350 to indicate the output of the alert(s) 1355, or a combination thereof. In some examples, the communication(s) sent from the portable dialysis system 1335 to the user device 1350 include the alert(s) 1355. In some examples, the user device 1350 can be a part of the portable dialysis system 1335. In some examples, the user device 1350 can be a distinct device from the portable dialysis system 1335.

In some examples, the alert(s) 1355 can indicate adjustment(s) (e.g., adjustment(s) 1435) that the patient 1325 should make to the portable dialysis system 1335 (e.g., an instruction or request or command to the patient 1325 to make the adjustment(s)), adjustment(s) (e.g., adjustment(s) 1435) that the patient 1325 should make to their lifestyle (e.g., an instruction or request or command to the patient 1325 to make the adjustment(s)), adjustment(s) (e.g., adjustment(s) 1435) that the portable dialysis system 1335 has automatically made or will automatically make (e.g., based on confirmation from the patient 1325 or in some cases even without confirmation from the patient 1325), determinations or predictions (e.g., alert(s) 1440) that the portable dialysis system 1335 has made (e.g., based on sensor data and/or other context data 1410), or a combination thereof.

For instance, examples of the alert(s) 1355 can include an alert that the filter (e.g., filter(s) 120) needs to be replaced (e.g., instructing the patient 1325 or another user to make this adjustment), an alert reminding the patient 1325 to drink more water (e.g., instructing the patient 1325 make this lifestyle adjustment based on sensor data indicating insufficient water in the peritoneal cavity 190), an alert that a supply of one or more electrolytes or other substances (e.g., used by the mixer 130) need to be replaced (e.g., instructing the patient 1325 or another user to make this adjustment), an alert warning the user that one or more early signs of infection were detected or the probability of having a peritonitis within given amount of time will be presented (e.g., based on the sensor data and/or other context data 1410), an alert that a position and/or orientation of the catheter (e.g., catheter 165, catheter 1320, catheter 1340) needs to be adjusted (e.g., instructing the patient 1325 or another user to make this adjustment), an alert that the waste container is full and should be drained and/or otherwise evacuated (e.g., instructing the patient 1325 or another user to make this adjustment), an alert that settings of the portable dialysis system 1335 should be changed (e.g., instructing the patient 1325 or another user to make this adjustment), an alert that settings of the portable dialysis system 1335 have automatically been changed or will automatically be changed (e.g., informing the 1325 or another user that the change has been made or asking for permission and/or confirmation to make this change), an alert that the dialysate quality is poor (e.g., instructing the patient 1325 or another user to check the portable dialysis system 1335 for problems and/or take the portable dialysis system 1335 in for maintenance and/or repairs and/or other adjustments), power level, or a combination thereof. In some examples, the alert(s) 1355 can also include any other alerts, notifications, and/or warnings of any other information, determinations and/or predictions (e.g., alert(s) 1440), adjustment(s) (e.g., adjustment(s) 1435), or a combination thereof.

FIG. 14 is a block diagram illustrating an example of a machine learning system 1400 for training and use of one or more machine learning model(s) 1425 in a peritoneal dialysis system. The machine learning system 1400 a machine learning (ML) engine 1420 that generates, trains, uses, and/or updates one or more ML model(s) 1425. In some examples, the machine learning system 1400, or at least one component thereof, can be part of the controller 160, can be run on the controller 160, can be coupled to the controller 160, can be networked with the controller 160, can be in communication (e.g., through a wired or wireless communication interface) with the controller 160, or a combination thereof.

The ML model(s) 1425 can include, for instance, one or more neural network (NN(s)), convolutional NN(s) (CNN(s)), trained time delay NN(s) (TDNN(s)), deep network(s), autoencoder(s) (AE(s)), variational AE(s) (VAE(s)), deep belief net(s) (DBN(s)), recurrent NN(s) (RNN(s)), generative adversarial network(s) (GAN(s)), conditional GAN(s) (cGAN(s)), support vector machine(s) (SVM(s)), random forest(s) (RF(s)), decision tree(s), NN(s) with fully connected (FC) layer(s), NN(s) with convolutional layer(s), computer vision (CV) system(s), decp learning (DL) system(s), classifier(s), transformer(s), clustering algorithm(s), gradient boosting model(s), sequence-to-sequence (Seq2Seq) model(s), autoregressive (AR) model(s), large language model(s) (LLMs), model(s) trained using genetic algorithm(s) (GA(s)), model(s) trained using evolutionary algorithm(s) (EA(s)), model(s) trained using neuroevolution of augmenting topologies (NEAT) algorithm(s), model(s) trained using deep Q learning (DQN) algorithm(s), model(s) trained using advantage actor-critic (A2C) algorithm(s), model(s) trained using proximal policy optimization (PPO) algorithm(s), model(s) trained using reinforcement learning (RL) algorithm(s), model(s) trained using supervised learning (SL) algorithm(s), model(s) trained using unsupervised learning (UL) algorithm(s), or combinations thereof. Examples of LLMs that can be used can include, for instance, Generative Pre-Trained Transformer (GPT) (e.g., GPT-2, GPT-3, GPT-3.5, GPT-4, ChatGPT, and/or other GPT variant(s)), DaVinci, LLMs using Massachusetts Institute of Technology (MIT) langchain, Google® Bard®, Google® Gemini®, Large Language Model Meta AI (LLaMA), LLAMA 2, LLAMA 3, LLAMA 4, Megalodon, or combinations thereof.

Within FIG. 14, a graphic representing the ML model(s) 1425 illustrates a set of circles connected to one another. Each of the circles can represent a node, a neuron, a perceptron, a layer, a portion thereof, or a combination thereof. The circles are arranged in columns. The leftmost column of white circles represent an input layer. The rightmost column of white circles represent an output layer. Two columns of shaded circled between the leftmost column of white circles and the rightmost column of white circles each represent hidden layers. An ML model can include more or fewer hidden layers than the two illustrated, but includes at least one hidden layer. In some examples, the layers and/or nodes represent interconnected filters, and information associated with the filters is shared among the different layers with each layer retaining information as the information is processed. The lines between nodes can represent node-to-node interconnections along which information is shared. The lines between nodes can also represent weights (e.g., numeric weights) between nodes, which can be tuned, updated, added, and/or removed as the ML model(s) 1425 are trained and/or updated. In some cases, certain nodes (e.g., nodes of a hidden layer) can transform the information of each input node by applying activation functions (e.g., filters) to this information, for instance applying convolutional functions, downscaling, upscaling, data transformation, and/or any other suitable functions.

In some examples, the ML model(s) 1425 can include a feed-forward network, in which case there are no feedback connections where outputs of the network are fed back into itself. In some cases, the ML model(s) 1425 can include a recurrent neural network, which can have loops that allow information to be carried across nodes while reading in input. In some cases, the network can include a convolutional neural network, which may not link every node in one layer to every other node in the next layer.

One or more input(s) 1405 can be provided to the ML model(s) 1425. The ML model(s) 1425 can be trained by the ML engine 1420 (e.g., based on training data 1460) to generate one or more output(s) 1430.

In some examples, the input(s) 1405 include context data 1410. The context data 1410 can include information about a peritoneal dialysis system (e.g., peritoneal dialysis system 105, portable dialysis system 1315, portable dialysis system 1335, the peritoneal dialysis system that performs the process 1500), such as sensor data from any of the sensors of the peritoneal dialysis system, such as the sensor(s) 135, the sensor(s) S_{A} 230, the sensor(s) S_{B} 235, the first lumen sensor(s) 270, the second lumen sensor(s) 275, the sensor(s) S_{A} 330, the sensor(s) S_{B} 335, the lumen sensor 370, the sensor(s) S_{A} 405, the sensor(s) S_{B} 410, the sensor(s) S_{C} 415, the first lumen sensor(s) 470, the second lumen sensor(s) 475, the third lumen sensor(s) 480, sensor(s) in the sorter 540, lumen sensor(s) coupled to the tip(s) of the outflow lumens 550, lumen sensor(s) coupled to the tip(s) of the inflow lumens 555, the sensor(s) 660, the sensor(s) 665, the sensor(s) 760A-760C, the sensor(s) 870A-870D, any sensor(s) associated with any of the sensor data from the table 900, any sensor(s) associated with any of the sensor data from the table 1000, any sensor(s) associated with any of the sensor data from the table 1100, one or more sensors that capture volume measurement data (as in the volume graph 905), one or more sensors that capture flow measurement data (as in the flow graph 910), any sensor(s) of the portable dialysis system 1315, any sensor(s) of the portable dialysis system 1335, any sensor(s) (e.g., input device 1645) of the computing system 1600, or a combination thereof. In some cases, the sensor data from the sensor(s) of the peritoneal dialysis system can include conductivity data from conductivity sensor(s), pH data from pH sensor(s), chemical concentration data from chemical concentration sensor(s), particulate concentration data from particulate sensor(s), flow data from flow sensor(s), volume data from volume sensor(s), pressure data (e.g., hydrostatic pressure data) from pressure sensors (e.g., hydrostatic pressure sensors), temperature data from temperature sensors (e.g., thermometers, thermistors, temperature transducers), image data from camera(s), audio data from microphone(s), a pose (e.g., location and/or orientation) of the peritoneal dialysis system (e.g., as determined using pose sensor(s) of the peritoneal dialysis system), or a combination thereof.

Within the context data 1410, the information about a peritoneal dialysis system can also include settings of the peritoneal dialysis system, such as filtration settings (e.g., of the filter(s) 120 and/or the filter subsystem 605, for instance indicating how many times the fluid 185 should pass through the second sensor usability indicator 640 and/or the recirculation loop 615), valve settings (e.g., open, closed, or partially open/partially closed) for one or more valves (e.g., valve V_{A} 205, valve V_{B} 210, valve V_{C} 215, valve V_{D} 220, valve V_{A} 305, valve V_{B} 310, division valve 620, directional valve 625, directional valve 635), pump settings (e.g., on, off, pump strength) for one or more pumps (e.g., outflow pump(s) Pₒ 260, inflow pump(s) Pᵢ 265, pump(s) P_{A} 505, pump(s) P_{B} 510, pump(s) P_{C} 515, pump(s) PD 520, the pump 630, the pump 850, a pump that conveys waste from the filter(s) 120 to the waste container 125 and/or waste evaporator subsystem 805), mixer settings (e.g., which substances to add to the filtered fluid to generate the dialysate, an amount of each of the substances to add to the filtered fluid to generate the dialysate), rules for approval of dialysate (e.g., one or more dialysate characteristic thresholds for comparing one or more characteristics of the dialysate measured by the sensor(s) 135 to), which lumen(s) are dependent relative to which other lumen(s), which lumen(s) are designated (e.g., by the controller 160) as outflow lumen(s), which lumen(s) are designated (e.g., by the controller 160) as inflow lumen(s), other settings discussed herein, or a combination thereof. Within the context data 1410, the information about a peritoneal dialysis system can also include when the peritoneal dialysis system was first used with the patient, when the peritoneal dialysis system was last serviced, when settings of the peritoneal dialysis system were last adjusted, when components and/or consumables of the peritoneal dialysis system were last replaced (e.g., filters of the filter(s) 120, substances that the mixer 130 adds to the filtered fluid to generate the dialysate, etc.), when the peritoneal dialysis system was first hooked up to the patient, when the closed circuit of the peritoneal dialysis system was last broken (e.g., to disconnect the peritoneal dialysis system from the patient and/or reconnect the peritoneal dialysis system to the patient), and/or record(s) of other events associated with the peritoneal dialysis system.

The context data 1410 can include information about a patient, such as a sex of the patient, a gender of the patient, an age of the patient, a race of the patient, an ethnic background of the patient, a weight of the patient, a height of the patient, genetic data associated with the patient, any diseases that the patient has, any dysfunction(s) that the patient has, the patient's water intake, the patient's food intake, the patient's medication intake, the patient's fitness level, the patient's fitness activity, any medication(s) that the patient is taking or has taken, any predisposition(s) (e.g., to diseases, disorders, or dysfunctions) that the patient has (e.g., genetic predisposition(s), family history), family history of the patient, patient demographic data, patient medical records, patient laboratory data, clinical notes, clinical findings, imaging reports, physician examination data (e.g., temperature, weight, diagnoses, notes), patient history, patient symptoms, diagnoses, patient biopsy results, impedance, any other patient data discussed herein, data recording any infection(s) suffered by the patient (e.g., infections associated with the peritoneal dialysis system or otherwise), information about the schedule of the patient (e.g., scheduled maintenance of the peritoneal dialysis system and/or scheduled doctor's appointment(s)), a pose (e.g., location and/or orientation) of the patient (e.g., as determined using pose sensor(s) of the peritoneal dialysis system and/or a user device), other patient data discussed herein, or a combination thereof. The context data 1410 can include user interface inputs that are input into, and received by, a user interface of the peritoneal dialysis system (or another system such as the user device 1350 or a physician system of a physician) by the patient, a physician, a doctor, a registered nurse (RN), a nurse practitioner (NP), a family member of the patient, a technician, a maintenance worker, an operator of the peritoneal dialysis system 105, or another user. The context data 1410 can also include other contextual information, such as a current time and/or a current date.

In some examples, the input(s) 1405 can include previous output(s) 1415, such as previous iterations of the output(s) 1430 generated by the ML model(s) 1425 (e.g., previous iterations of the adjustment(s) 1435 and/or the alert(s) 1440). In some examples, the input(s) 1405 can include partially-processed data that is to be processed further, such as various features, weights, intermediate data, layer data from specific layer(s) of the ML model(s) 1425, or a combinations thereof.

The output(s) 1430 generated by the ML model(s) 1425 in response to input of the input(s) 1405 (e.g., in response to the context data 1410 and/or the previous output(s) 1415) into the ML model(s) 1425 can include adjustment(s) 1435 to make to the peritoneal dialysis system to improve operation of the peritoneal dialysis system. The adjustment(s) 1435 can include adjustment(s) that the patient or another user should make to the portable dialysis system (e.g., an instruction or request or command to the patient or the other user to make the adjustment(s)), adjustment(s) that the patient should make to their lifestyle (e.g., an instruction or request or command to the patient to make the adjustment(s)), adjustment(s) that the portable dialysis system has automatically made or will automatically make (e.g., after receiving permission and/or confirmation from the patient, or in some cases even without permission or confirmation from the patient), or a combination thereof.

In some examples, the adjustment(s) 1435 can include an adjustment to the filter(s) 120 and/or the filter subsystem 605, for instance to replace a filter (e.g., instructing the patient or another user to make this adjustment). In some examples, the adjustment(s) 1435 can include an adjustment to the add more to the reserves of substances (stored in the peritoneal dialysis system 105 or in storage containers coupled to the peritoneal dialysis system 105) (e.g., cartridge 710 of substance 715, cartridge 720 of substance 725, cartridge 730 of substance 735) that the mixer 130 (e.g., mixer subsystem 705) uses to add to the filtered fluid to generate the dialysate, for instance because the reserves are running low (e.g., as determined based on sensor(s) 760A-760C monitoring an amounts of the reserves) (e.g., instructing the patient or another user to make this adjustment). For instance, the adjustment(s) 1435 can include an adjustment to the add more sodium (Na⁺), potassium (K⁺), chloride (Cl⁻), bicarbonate, calcium (Ca²⁺), magnesium (Mg²⁺), phosphate, other electrolytes, glucose, dextrose, lactate, acetate, other non-electrolytes, salts (e.g., NaCl, KCl), other solutes mentioned herein, other substances mentioned herein, or a combination thereof. In some examples, the adjustment(s) 1435 can include an adjustment to certain settings and/or modes of the peritoneal dialysis system, for instance to set or change the settings and/or modes (e.g., instructing the patient or another user to make this adjustment).

In some examples, the adjustment(s) 1435 can include an adjustment to the catheter (e.g., catheter 165, catheter 1320, catheter 1340) and/or lumen(s), such as an adjustment to the position and/or orientation of the catheter (e.g., instructing the patient or another user to make this adjustment, or actuating an actuator to automatically make the adjustment). In some examples, the adjustment(s) 1435 can include an adjustment to the waste container 125 and/or waste evaporator subsystem 805, for instance to drain or otherwise evacuate the waste container 125 and/or waste evaporator subsystem 805 because the waste container 125 and/or waste evaporator subsystem 805 is storing over a threshold amount of waste (e.g., is full or almost full) (e.g., instructing the patient or another user to make this adjustment). In some examples, the adjustment(s) 1435 can include repairs and/or maintenance actions (e.g., instructing the patient or another user to take the peritoneal dialysis system 105 in for maintenance or repairs, and/or instructing a repairs or maintenance on which repairs or maintenance actions to perform, to make the adjustment).

In some examples, the adjustment(s) 1435 can include adjustment(s) to any of the setting(s) and/or mode(s) (e.g., the divided inflow/outflow mode, the multifunctional lumen mode) of the peritoneal dialysis system (e.g., instructing the patient or another user to make this adjustment, or automatically making the adjustment). The settings to be adjusted according to the adjustment(s) 1435 can include, for instance, filtration settings (e.g., of the filter(s) 120 and/or the filter subsystem 605, for instance indicating how many times the fluid 185 should pass through the second sensor usability indicator 640 and/or the recirculation loop 615), valve settings (e.g., open, closed, or partially open/partially closed) for one or more valves (e.g., valve V_{A} 205, valve V_{B} 210, valve V_{C} 215, valve V_{D} 220, valve V_{A} 305, valve V_{B} 310, division valve 620, directional valve 625, directional valve 635), pump settings (e.g., on, off, pump strength) for one or more pumps (e.g., outflow pump(s) Pₒ 260, inflow pump(s) Pᵢ 265, pump(s) P_{A} 505, pump(s) P_{B} 510, pump(s) P_{C} 515, pump(s) PD 520, the pump 630, the pump 850, a pump that conveys waste from the filter(s) 120 to the waste container 125 and/or waste evaporator subsystem 805), mixer settings (e.g., which substances to add to the filtered fluid to generate the dialysate, an amount of each of the substances to add to the filtered fluid to generate the dialysate), rules for approval of dialysate (e.g., one or more dialysate characteristic thresholds for comparing one or more characteristics of the dialysate measured by the sensor(s) 135 to), which lumen(s) are dependent relative to which other lumen(s), which lumen(s) are designated (e.g., by the controller 160) as outflow lumen(s), which lumen(s) are designated (e.g., by the controller 160) as inflow lumen(s), other settings discussed herein, or a combination thereof.

In some examples, the adjustment(s) 1435 can include an adjustment to the lifestyle of the patient, for instance an adjustment to the patient's water intake (e.g., reminding the patient to drink more water or drink less water to make this adjustment), the patient's food intake (e.g., reminding the patient to eat more food or cat less food to make this adjustment), the patient's medicine intake (e.g., reminding the patient to take more of a medicine or take less of the medicine to make this adjustment), and/or the patient's fitness (e.g., reminding the patient to exercise more or less to make this adjustment),

The output(s) 1430 generated by the ML model(s) 1425 in response to input of the input(s) 1405 (e.g., in response to the context data 1410 and/or the previous output(s) 1415) into the ML model(s) 1425 can include alert(s) 1440. The term alert(s) 1440, as used in the context of FIG. 14, can refer to various determinations, estimates, indications, and/or predictions that the ML model(s) 1425 have made based on the input(s) 1405. For instance, in some examples, the alert(s) 1440 can include an indication that one or more signs of infection (e.g., drop in pH, rise in pH, temperature, and/or conductivity in the peritoneal cavity 190, and/or increase in concentration of chemical(s) associated with infection in the peritoneal cavity 190) were detected (e.g., by the lumen sensor(s) and/or sensor(s) that monitor the fluid 185 from the peritoneal cavity 190 such as the sensor(s) S_{B} 235 in FIG. 2A, the sensor(s) S_{A} 230 in FIG. 2B, the sensor(s) S_{B} 335, or the sensor(s) S_{C} 415).

In some examples, the alert(s) 1440 can include alerts notifying that the adjustment(s) 1435 are to be implemented by the patient or another user, alerts notifying that the adjustment(s) 1435 have been implemented automatically by the peritoneal dialysis system, and/or alerts asking the patient or another user for permission and/or confirmation to implement the adjustment(s) 1435. For instance, the alert(s) 1440 can inform the patient or other user that changing a specific setting of the peritoneal dialysis system would improve operation of the peritoneal dialysis system, can instruct the patient or other user on how to change the specific setting, can confirm that the patient or other user has correctly changed the specific setting, can ask the patient or other user for permission or confirmation to automatically change the specific setting, can inform the patient or other user that the peritoneal dialysis system has automatically changed the specific setting, or a combination thereof.

In some examples, the alert(s) 1440 can include alerts about the conditions leading to the suggestion of, or implementation of, any of the adjustment(s) 1435 discussed herein. For instance, the alert(s) 1440 can identify that, according to the sensor data from the sensor(s) of the peritoneal dialysis system (e.g., sensor(s) 135), a quality of the dialysate is low (e.g., includes a concentration of waste products that is above a maximum threshold, includes a concentration of electrolytes or other desirable substances that is below a minimum threshold, and the like), with this being a condition corresponding to suggested adjustment(s) (e.g., adjustment(s) 1435) to replace the filter(s) (e.g., of the filter(s) 120 and/or the filter subsystem 605), to refill reservoirs of the substances used by the mixer 130, to change setting(s) of the peritoneal dialysis system, and/or to bring the peritoneal dialysis system in for maintenance and/or repairs. The alert(s) 1440 can identify that issues with the patient's lifestyle have been detected, such as issues with the patient's water intake (e.g., below a minimum threshold or above a maximum threshold), issues with the patient's food intake (e.g., below a minimum threshold or above a maximum threshold), issues with the patient's medicine intake (e.g., below a minimum threshold or above a maximum threshold), issues with the patient's fitness activity (e.g., below a minimum threshold or above a maximum threshold), or a combination thereof.

In some examples, the ML system that includes the ML engine 1420 and/or ML model(s) 1425 adds the output(s) 1430 to a data store (e.g., the data store(s) 280). Data can be drawn from these data store(s) to use as input(s) 1405 for the ML model(s) 1425 for generating future output(s) 1430 (e.g., as the previous output(s) 1415).

In some examples, the ML system repeats the process illustrated in FIG. 14 multiple times to generate the output(s) 1430 in multiple passes, using some of the output(s) 1430 from earlier passes as some of the input(s) 1405 in later passes (e.g., as the previous output(s) 1415). For instance, in an illustrative example, in a first pass, the ML model(s) 1425 can process the context data 1410 to generate adjustment(s) 1435 and/or alert(s) 1440. In a second pass, the ML model(s) 1425 can add (e.g., append) the adjustment(s) and/or alert(s) from the first pass to the input(s) 1405 (e.g., as the previous output(s) 1415), and can generate further adjustment(s) 1435 and/or alert(s) 1440. In some examples, the further adjustment(s) 1435 and/or alert(s) 1440 (generated in the second pass) can be influenced by the adjustment(s) and/or alert(s) (generated in the first pass), for instance with implementation of the adjustment(s) in the first pass potentially causing further adjustment(s) to be recommended in the second pass, and/or causing other actions to occur that provoke the alert(s) 1440 in the second pass. In some cases, the further adjustment(s) 1435 and/or alert(s) 1440 (generated in the second pass) can also be based on additional context data 1410, such as sensor data (or other types of context data 1410) received after the adjustment(s) (from the first pass) are implemented and/or after the alert(s) (from the first pass) are output at the user device.

In some examples, the context data 1410 continues to be updated dynamically and in real-time, for instance with sensor data continuing to be received over time from the various sensors of the peritoneal dialysis system. In some examples, the ML model(s) 1425 can update the output(s) 1430 dynamically and in real-time based on updates to the context data 1410 as the updates to the context data 1410 continue to be received.

In some examples, the ML system includes one or more feedback engine(s) 1445 that generate and/or provide feedback 1450 about the output(s) 1430. In some examples, the feedback 1450 indicates how well the output(s) 1430 align to corresponding expected output(s), how well the output(s) 1430 serve their intended purpose, or a combination thereof. In some examples, the feedback engine(s) 1445 include loss function(s), reward model(s) (e.g., other ML model(s) that are used to score the output(s) 1430), discriminator(s), error function(s) (e.g., in back-propagation), user interface feedback received via a user interface from a user, or a combination thereof. In some examples, the feedback 1450 can include one or more alignment score(s) that score a level of alignment between the output(s) 1430 and the expected output(s) and/or intended purpose.

The ML engine 1420 of the ML system can update (further train and/or fine-tune) the ML model(s) 1425 based on the feedback 1450 to perform an update 1455 (e.g., further training) of the ML model(s) 1425 based on the feedback 1450. In some examples, the feedback 1450 includes positive feedback, for instance indicating that the output(s) 1430 closely align with expected output(s) and/or that the output(s) 1430 serve their intended purpose. In some examples, the feedback 1450 includes negative feedback, for instance indicating a mismatch between the output(s) 1430 and the expected output(s), and/or that the output(s) 1430 do not serve their intended purpose. For instance, high amounts of loss and/or error (e.g., exceeding a threshold) can be interpreted as negative feedback, while low amounts of loss and/or error (e.g., less than a threshold) can be interpreted as positive feedback. Similarly, high amounts of alignment (e.g., exceeding a threshold) can be interpreted as positive feedback, while low amounts of alignment (e.g., less than a threshold) can be interpreted as negative feedback. In response to positive feedback in the feedback 1450, the ML engine 1420 can perform the update 1455 to update the ML model(s) 1425 to strengthen and/or reinforce weights associated with generation of the output(s) 1430 to encourage the ML engine 1420 to generate similar output(s) 1430 given similar input(s) 1405, thereby improving accuracy of future output(s) 1430 generated by the ML model(s) 1425. In response to negative feedback in the feedback 1450, the ML engine 1420 can perform the update 1455 to update the ML model(s) 1425 to weaken and/or remove weights associated with generation of the output(s) 1430 to discourage the ML engine 1420 from generating similar output(s) 1430 given similar input(s) 1405, thereby improving accuracy of future output(s) 1430 generated by the ML model(s) 1425.

In some examples, the update 1455 can set and/or modify certain hyperparameters, settings, options, and/or parameters of the ML model(s) 1425, such as temperature (e.g., level of creativity) for the output(s) 1430, a top P value (e.g., influencing creativity level and/or randomness level) for the output(s) 1430, a frequency penalty (e.g., to prevent output(s)) for the output(s) 1430, a presence penalty (e.g., to encourage the personalized ML model(s) 1425 to introduce new output(s)) for the output(s) 1430, other hyperparameters discussed herein, other settings discussed herein, other options discussed herein, other parameters discussed herein, or a combination thereof. In some examples, the update 1455 can set and/or modify certain hyperparameters, settings, options, and/or parameters of the ML model(s) 1425 based on the feedback 1450, for instance if the feedback 1450 indicates whether the output(s) 1430 in one pass match, or are different from, the output(s) 1430 in another pass (e.g., indicative of randomness and/or frequency penalty and/or presence penalty).

In an illustrative example, if the ML model(s) 1425 generates adjustment(s) 1435 based on the context data 1410, and the feedback 1450 indicates that the performance of the peritoneal dialysis system ultimately did improve after the adjustment(s) 1435 were implemented and/or the alert(s) 1440 were output (e.g., the quality of the dialysate improved, the health of the patient improved, or the like), then the feedback 1450 can be interpreted as positive feedback, strengthening the weights of the ML model(s) 1425 that were responsible for generating the output(s) 1430 to encourage generation of similar output(s) 1430 given similar input(s) 1405 to improve the quality of further output(s) 1430 generated by the ML model(s) 1425.

On the other hand, if the feedback 1450 indicates that the performance of the peritoneal dialysis system ultimately did not improve after the adjustment(s) 1435 were implemented and/or the alert(s) 1440 were output (e.g., the quality of the dialysate did not improve, the health of the patient did not improve, or the like), or that the performance of the peritoneal dialysis system ultimately got worse after the adjustment(s) 1435 were implemented and/or the alert(s) 1440 were output (e.g., the quality of the dialysate got worse, the health of the patient got worse, or the like), then the feedback 1450 can be interpreted as negative feedback, weakening or removing the weights of the ML model(s) 1425 that were responsible for generating the adjustment(s) 1435 to discourage generation of similar output(s) 1430 given similar input(s) 1405 to improve the quality of further output(s) 1430 generated by the ML model(s) 1425.

In some examples, the ML engine 1420 can also perform an initial training of the ML model(s) 1425 before the ML model(s) 1425 are used to generate the output(s) 1430 based on the input(s) 1405. During the initial training, the ML engine 1420 can train the ML model(s) 1425 based on training data 1460. In some examples, the training data 1460 includes examples of input(s) (of any input types discussed with respect to the input(s) 1405), output(s) (of any output types discussed with respect to the output(s) 1430), and/or feedback (of any feedback types discussed with respect to the feedback 1450). In an illustrative example, the training data 1460 can include examples of context data (e.g., as in context data 1410), previous output(s) (e.g., as in previous output(s) 1415), adjustment(s) (e.g., as in the adjustment(s) 1435), alert(s) (e.g., as in the alert(s) 1440), and/or feedback (e.g., as in the feedback 1450). In some cases, positive feedback in the training data 1460 can be used to perform positive training, to encourage the ML model(s) 1425 to generate output(s) similar to the output(s) in the training data given input of the corresponding input(s) in the training data to improve the ability of the ML model(s) 1425 to generate further output(s) well. In some cases, negative feedback in the training data 1460 can be used to perform negative training, to discourage the ML model(s) 1425 from generate output(s) similar to the output(s) in the training data given input of the corresponding input(s) in the training data to improve the ability of the ML model(s) 1425 to generate further output(s) well. In some examples, training (e.g., initial training with training data 1460 and/or the update(s) 1455) can be performed in a cloud computing environment. In some examples, the ML model(s) 1425 can be trained to be specific to a specific patient, for instance using, as training data 1460, patient data, sensor data and/or other context data 1410 that is specific to that patient (and/or their peritoneal dialysis system 105). In some examples, the ML model(s) 1425 can be trained on training data corresponding to multiple patients, for instance using, as training data 1460, patient data, sensor data and/or other context data 1410 from across multiple patients and/or their respective peritoneal dialysis systems. In some examples, the multiple patients can be selected based on similarity to one another, and/or to a specific patient (e.g., sharing a disease, a symptom, a sex, an age, a genetic disposition, a type of dialysis device, or other attribute(s)). In some examples, some or all of the patient data can be synthetic, for instance based on synthetic patient data that is generated using the ML model(s) 1425 and/or other ML model(s) based on distributions of possible values. In some examples, the ML engine 1420 can train the ML model(s) 1425 on a specific patient, a patient's history, and/or multiple patients using similar or different dialysis modalities, and/or synthetic patient data, or a combination thereof.

In some examples, the ML model(s) 1425 can generate the output(s) 1430 dynamically and in real-time as the input(s) 1405 continue to be received by the ML model(s) 1425. This can ensure that the output(s) 1430 are generated based on up-to-date input(s) 1405.

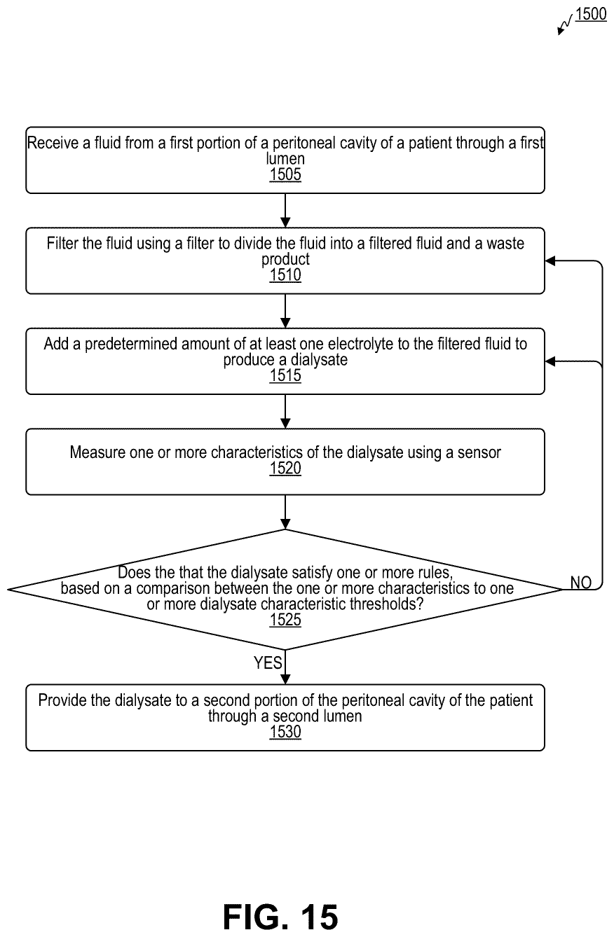

FIG. 15 is a flow diagram illustrating a process 1500 for peritoneal dialysis. The process 1500 can be performed by, and/or using, a peritoneal dialysis system. The peritoneal dialysis system can include, for instance, the peritoneal dialysis system 105 of any of FIGS. 1-5, the filter subsystem 605, the waste evaporator subsystem 805, the portable dialysis system 1315, the portable dialysis system 1335, the user device 1350, the machine learning engine 1420, the ML model(s) 1425, the feedback engine(s) 1445, the computing system 1600, a system, an apparatus, a point of sale (POS) system or terminal, a medical device, a dialysis system, a processor that performs instructions stored in a non-transitory computer-readable storage medium, any subsystems or components of any of the above-listed systems, any other computing systems discussed herein, or a combination thereof.

At operation 1505, the peritoneal dialysis system (or a subsystem thereof) is configured to, and can, receive a fluid (e.g., fluid 185) from a first portion of a peritoneal cavity (e.g., the peritoneal cavity 190) of a patient (e.g., patient 195, patient 1305, patient 1325) through a first lumen. Examples of the first lumen include the outflow lumen 150 of FIG. 1, the second lumen 255 of FIG. 2A, the first lumen 250 of FIG. 2B, the lumen 350, the third lumen 460, the outflow lumens 550, or a combination thereof. In some examples, the first portion of the peritoneal cavity is an area at or near (e.g., within a radius of) a tip of the first lumen.

In some examples, at least one pump (e.g., outflow pump(s) Pₒ 260, inflow pump(s) Pᵢ 265, pump(s) P_{A} 505, pump(s) P_{B} 510, pump(s) P_{C} 515, pump(s) PD 520, the pump 630, the pump 850) drives (e.g., pulls, suctions, conveys, retrieves) the fluid (e.g., fluid 185) from the first portion of the peritoneal cavity (e.g., the peritoneal cavity 190) through the first lumen and toward other component(s) of the peritoneal dialysis system (e.g., recycler 110, sorter 115, sorter 240, sorter 340, sorter 440, sorter 540).

At operation 1510, the peritoneal dialysis system (or a subsystem thereof) is configured to, and can, filter the fluid using a filter (e.g., filter(s) 120, filter subsystem 605, filter(s) 640) to divide the fluid into a filtered fluid (e.g., filtered fluid 685, filtered fluid 785) and a waste product (e.g., waste output 650).

In some examples, the filter of operation 1510 includes a reverse osmosis (RO) filter. In some examples, the peritoneal dialysis system includes a recirculation loop (e.g., recirculation loop 615) that passes at least a subset of the fluid through the filter (e.g., filter(s) 640) at least twice.

At operation 1515, the peritoneal dialysis system (or a subsystem thereof) is configured to, and can, add (e.g., using the mixer 130 and/or the mixer subsystem 705) a predetermined amount of at least one electrolyte (e.g., substance 715, substance 725, and/or substance 735) to the filtered fluid (e.g., filtered fluid 685, filtered fluid 785) to produce a dialysate (e.g., dialysate 790).

In some examples, the processor (e.g., controller 160) of the peritoneal dialysis system determines the predetermined amount (of operation 1515) based on sensor data from at last one of the sensor or a second sensor (e.g., any of the other examples of the sensor discussed herein, and/or any other context data 1410 discussed herein). For instance, in some examples, the predetermined amount can be determined by the ML model(s) 1425 based on the input(s) 1405.

At operation 1520, the peritoneal dialysis system (or a subsystem thereof) is configured to, and can, measure one or more characteristics of the dialysate using a sensor.

In some examples, the sensor of operation 1520 is one of the sensor(s) 135. In some examples, the sensor of operation 1520 is any one of the sensor(s) 135, the sensor(s) S_{A} 230, the sensor(s) S_{B} 235, the first lumen sensor(s) 270, the second lumen sensor(s) 275, the sensor(s) S_{A} 330, the sensor(s) S_{B} 335, the lumen sensor 370, the sensor(s) S_{A} 405, the sensor(s) S_{B} 410, the sensor(s) S_{C} 415, the first lumen sensor(s) 470, the second lumen sensor(s) 475, the third lumen sensor(s) 480, sensor(s) in the sorter 540, lumen sensor(s) coupled to the tip(s) of the outflow lumens 550, lumen sensor(s) coupled to the tip(s) of the inflow lumens 555, the sensor(s) 660, the sensor(s) 665, the sensor(s) 760A-760C, the sensor(s) 870A-870D, any sensor(s) associated with any of the sensor data from the table 900, any sensor(s) associated with any of the sensor data from the table 1000, any sensor(s) associated with any of the sensor data from the table 1100, one or more sensors that capture volume measurement data (as in the volume graph 905), one or more sensors that capture flow measurement data (as in the flow graph 910), any sensor(s) of the portable dialysis system 1315, any sensor(s) of the portable dialysis system 1335, any sensor(s) (e.g., input device 1645) of the computing system 1600, or a combination thereof.

In some examples, the at least one electrolyte (of operation 1515) includes sodium (e.g., Na⁺). In some examples, the predetermined amount (of operation 1515) of the at least one electrolyte falls within a range of 130 to 150 milliEquivalents/Liter (mEq/L) of sodium, which can create an osmotic gradient and clear out waste from a patient regardless of the patient's sodium level. In some examples, the at least one electrolyte includes at least one of sodium (Na⁺), potassium (K⁺), chloride (Cl⁻), bicarbonate, calcium (Ca²⁺), magnesium (Mg²⁺), phosphate, other electrolytes, glucose, dextrose, lactate, acetate, other non-electrolytes, salts (e.g., NaCl, KCl), other solutes mentioned herein, other substances mentioned herein, or a combination thereof. In some examples, the at least one electrolyte can be used to treat sodium and/or electrolyte disturbances, dehydration, and/or fluid overload states (e.g., heart failure), not only for renal failure.

In some examples, the one or more characteristics of the dialysate measured using the sensor in operation 1520 can include conductivity data from conductivity sensor(s), pH data from pH sensor(s), chemical concentration data from chemical concentration sensor(s), particulate concentration data from particulate sensor(s), flow data from flow sensor(s), volume data from volume sensor(s), pressure data (e.g., hydrostatic pressure data) from pressure sensors (e.g., hydrostatic pressure sensors), temperature data from temperature sensors (e.g., thermometers, thermistors, temperature transducers), image data from camera(s), audio data from microphone(s), or a combination thereof.

At operation 1525, the peritoneal dialysis system (or a subsystem thereof) is configured to, and can, compare the one or more characteristics to one or more dialysate characteristic thresholds to verify whether the dialysate satisfies one or more rules. If the dialysate satisfies the one or more rules (e.g., the one or more characteristics compare favorably to one or more dialysate characteristic thresholds), the peritoneal dialysis system (or a subsystem thereof) is configured to, and can, proceed to operation 1530. If the dialysate does not satisfy the one or more rules (e.g., the one or more characteristics does not compare favorably to one or more dialysate characteristic thresholds), the peritoneal dialysis system (or a subsystem thereof) is configured to, and can, return to operation 1510 (e.g., to perform further filtering of the dialysate), return to operation 1515 (e.g., to add more of the electrolyte and/or other substances such as water using the mixer 130), or a combination thereof.

In some examples, the one or more rules are satisfied when the one or more characteristics are greater than or equal to (meet or exceed) the one or more dialysate characteristic thresholds. For instance, one rule may be satisfied when a concentration of the at least one electrolyte (as one of the one or more characteristics) is greater than or equal to a concentration threshold (as one of the one or more dialysate characteristic thresholds). In some examples, the one or more rules are satisfied when the one or more characteristics are less than or equal to (meet or fall below) the one or more dialysate characteristic thresholds. For instance, another rule may be satisfied when a concentration of the at least one waste product (e.g., urea) (as one of the one or more characteristics) is less than or equal to a concentration threshold (as one of the one or more dialysate characteristic thresholds). In some examples, the one or more rules are satisfied when the one or more characteristics are within a dialysate characteristic range, where the range is between two dialysate characteristic thresholds. In some examples, the one or more rules are satisfied when the one or more characteristics are outside of a dialysate characteristic range, where the range is between two dialysate characteristic thresholds.

In some examples, verifying that the dialysate satisfies the one or more rules (of operation 1525) includes: determining that the one or more characteristics of the dialysate are greater than or equal to the one or more dialysate characteristic thresholds. In some examples, verifying that the dialysate satisfies the one or more rules (of operation 1525) includes: determining that the one or more characteristics of the dialysate are less than or equal to the one or more dialysate characteristic thresholds. In some examples, the one or more characteristics and/or the one or more dialysate characteristic thresholds are associated with at least one of conductivity, potential of hydrogen (pH), temperature, pressure, concentration of the at least one electrolyte in the dialysate, or concentration of at least one other substance in the dialysate.

At operation 1530, the peritoneal dialysis system (or a subsystem thereof) is configured to, and can, provide the dialysate (e.g., dialysate 790) to a second portion of the peritoneal cavity of the patient through a second lumen. Examples of the second lumen include the inflow lumen 155 of FIG. 1, the first lumen 250 of FIG. 2A, the second lumen 255 of FIG. 2B, the lumen 350, the first lumen 450, the second lumen 455, the inflow lumens 555, or a combination thereof. In some examples, the second portion of the peritoneal cavity is an area at or near (e.g., within a radius of) a tip of the second lumen.

In some examples, at least one pump (e.g., outflow pump(s) Pₒ 260, inflow pump(s) Pᵢ 265, pump(s) P_{A} 505, pump(s) P_{B} 510, pump(s) P_{C} 515, pump(s) PD 520, the pump 630, the pump 850) drives (e.g., pushes, expels, evacuates, conveys) the dialysate (e.g., dialysate 790) from portion(s) of the peritoneal dialysis system (e.g., recycler 110, mixer 130, sensor(s) 135, sorter 115, sorter 240, sorter 340, sorter 440, sorter 540) toward (and to) the second portion of the peritoneal cavity (e.g., the peritoneal cavity 190) through the second lumen.

In some examples, the peritoneal dialysis system includes a sorter (e.g., sorter 115, sorter 240, sorter 340, sorter 440, sorter 540) coupled to the first lumen and the second lumen. The sorter includes a plurality of valves (e.g., valve V_{A} 205, valve V_{B} 210, valve V_{C} 215, valve V_{D} 220, valve V_{A} 305, valve V_{B} 310, division valve 620, directional valve 625, directional valve 635). The processor (e.g., controller 160) sets respective states of each of the plurality of valves (e.g., open, closed, partially open, partially closed, passing fluid in a first direction, passing fluid in a second direction, dividing flow) to route the fluid (e.g., fluid 185) from the first lumen to a recycler (e.g., recycler 110) and to route the dialysate (e.g., dialysate 790) from the recycler (e.g., recycler 110) to the second lumen. The recycler includes the filter (e.g., filter(s) 120, filter subsystem 605), the mixer (e.g., mixer 130, mixer subsystem 705), and the sensor (e.g., sensor(s) 135).

In some examples, the first portion of the peritoneal cavity is further along a direction of gravity than the second portion of the peritoneal cavity. In other words, the first portion of the peritoneal cavity is lower down (in the direction of gravity) in the peritoneal cavity than the second portion of the peritoneal cavity. Thus, the first lumen is a dependent lumen compared to the second lumen.

In some examples, the second portion of the peritoneal cavity is further along a direction of gravity than the first portion of the peritoneal cavity. In other words, the second portion of the peritoneal cavity is lower down (in the direction of gravity) in the peritoneal cavity than the first portion of the peritoneal cavity. Thus, the second lumen is a dependent lumen compared to the first lumen.

In some examples, the peritoneal dialysis system includes a first lumen sensor coupled to the first lumen (e.g., at a tip or base) and a second lumen sensor coupled to the second lumen (e.g., at a tip or base). Examples of the first lumen sensor and/or the second lumen sensor include the sensor(s) S_{A} 230, the sensor(s) S_{B} 235, the first lumen sensor(s) 270, the second lumen sensor(s) 275, the sensor(s) S_{A} 330, the sensor(s) S_{B} 335, the lumen sensor(s) 370, the sensor(s) S_{A} 405, the sensor(s) S_{B} 410, the sensor(s) S_{C} 415, the first lumen sensor(s) 470, the second lumen sensor(s) 475, the third lumen sensor(s) 480, the sensor(s) in the sorter 540, the lumen sensors at the tips of the lumens of FIG. 5, or a combination thereof. In some examples, execution of the instructions by the processor (e.g., controller 160) causes the processor (e.g., controller 160) to determine (e.g., before operation 1505), based on lumen sensor data (received from the first lumen sensor and/or the second lumen sensor), that the tip of the first lumen is further along a direction of gravity than the tip of the second lumen (e.g., the first lumen tip is dependent relative to the second lumen's tip). In examples where the lumen sensors are on the tips of the lumens, this can be a determination that the first lumen sensor is further along a direction of gravity than the second lumen sensor. In some examples, the processor (e.g., controller 160) configures a sorter (e.g., sorter 115, sorter 240, sorter 340, sorter 440, sorter 540), based on the determination that the tip of the first lumen is further along the direction of gravity than the tip of the second lumen, to receive the fluid from the peritoneal cavity through the first lumen (as in operation 1505) and to provide the dialysate to the peritoneal cavity through the second lumen (as in operation 1530). In some examples, the determination that the tip of the first lumen is further along the direction of gravity than the tip of the second lumen is based on a first pressure measured by (a pressure sensor of) the first lumen sensor being higher than a second pressure measured by (a pressure sensor of) the second lumen sensor.

In some examples, the first lumen sensor is coupled to a base of the first lumen, and/or the second lumen sensor is coupled to a base of the second lumen. For instance, examples of the first lumen and/or second lumen in this arrangement can include the sensor(s) S_{A} 230, the sensor(s) S_{B} 235, the sensor(s) S_{A} 330, the sensor(s) S_{B} 335, the sensor(s) S_{A} 405, the sensor(s) S_{B} 410, the sensor(s) S_{C} 415, the sensor(s) in the sorter 540, or a combination thereof.

In some examples, the first lumen sensor is coupled to the tip of the first lumen, and/or the second lumen sensor is coupled to the tip of the second lumen. For instance, examples of the first lumen and/or second lumen in this arrangement can include the first lumen sensor(s) 270, the second lumen sensor(s) 275, the lumen sensor(s) 370, the first lumen sensor(s) 470, the second lumen sensor(s) 475, the third lumen sensor(s) 480, the lumen sensors at the tips of the lumens of FIG. 5, or a combination thereof. In some examples, the peritoneal dialysis system includes an inductive charger (e.g., charger 175) that provides a wireless power signal to the first lumen sensor and the second lumen sensor (e.g., while the first lumen sensor and the second lumen sensor are in the peritoneal cavity) to power the capture of the lumen sensor data by the first lumen sensor and the second lumen sensor. In some examples, wires run along the lumen(s) to provide power from the power supply 170 of the peritoneal dialysis system to the first lumen sensor and the second lumen sensor. In some examples, the peritoneal dialysis system includes a communication interface (e.g., wired communication interface and/or a wireless communication interface) that receives the lumen sensor data from the first lumen sensor and the second lumen sensor (e.g., as a wireless signal, as a wired signal from wire(s) that run along the lumen(s), or a combination thereof).

In some examples, based on additional sensor data received from the first lumen sensor and the second lumen sensor (e.g., after operation 1530), the processor (e.g., controller 160) determines the tip of the second lumen is further along the direction of gravity than the tip of the first lumen (e.g., reversing the previous arrangement of the first lumen sensor and the second lumen sensor). The processor (e.g., controller 160) can reconfigure the sorter, based on this determination, to receive the fluid from the peritoneal cavity through the second lumen and to provide the dialysate to the peritoneal cavity through the first lumen (e.g., the reverse lumen arrangement compared to operation 1505 and operation 1530). For instance, this reversal can be caused by movement of the lumens and/or of the patient, so that which lumen tip is dependent changes.

In some examples, based on additional sensor data received from the first lumen sensor and the second lumen sensor (e.g., after operation 1530), the processor (e.g., controller 160) determines that the tip of the first lumen and the tip of the second lumen are within a threshold distance of one another along the direction of gravity (e.g., are both a similar distance from ground level or sea level). A plurality of lumens includes the first lumen and the second lumen. The processor (e.g., controller 160) can reconfigure a sorter, based on this determination, to receive the fluid from the peritoneal cavity through at least one of the plurality of lumens (e.g., the first lumen and/or the second lumen) and to provide the dialysate to the peritoneal cavity through at least the one of the one of the plurality of lumens (e.g., the first lumen and/or the second lumen). For instance, this change can represent a change in mode from the divided inflow/outflow mode to the multifunctional lumen mode, and can occur when the peritoneal dialysis system cannot determine with confidence which lumen is dependent.

In some examples, the peritoneal dialysis system includes a waste container (e.g., waste container 125) that receives the waste product from the filter (e.g., filter(s) 120, filter subsystem 605, filter(s) 640). In some examples, the peritoneal dialysis system includes an evaporator (e.g., waste evaporator subsystem 805) that evaporates a liquid (e.g., liquid waste 815 evaporating into contaminated vapor 825) from the waste product to reduce a volume of the waste product (e.g., to solid waste 827) that is stored in the waste container.

In some examples, the peritoneal dialysis system includes a power supply (e.g., power supply 170) that provides power to at least the processor (e.g., controller 160), the memory, the sensor, the mixer, and at least one pump. The at least one pump drives the fluid from the peritoneal cavity through the first lumen. The at least one pump drives the dialysate to the peritoneal cavity through the second lumen. Examples of the at least one pump include the outflow pump(s) Pₒ 260, inflow pump(s) Pᵢ 265, pump(s) P_{A} 505, pump(s) P_{B} 510, pump(s) P_{C} 515, pump(s) PD 520, the pump 630, the pump 850, a pump that conveys waste from the filter(s) 120 to the waste container 125 and/or waste evaporator subsystem 805.

In some examples, the processor (e.g., controller 160) of the peritoneal dialysis system processes context data (e.g., context data 1410) using a trained machine learning model (e.g., ML model(s) 1425) to identify an adjustment (e.g., adjustment(s) 1435) to make to at least one component of the peritoneal dialysis system to improve a function (e.g., waste clearance, filtering, dialysate cleanliness, clearance speed, etc.) of the peritoneal dialysis system. In some examples, the processor (e.g., controller 160) of the peritoneal dialysis system automatically performs an action to make the adjustment (e.g., changes settings of the peritoneal dialysis system). In some examples, the processor (e.g., controller 160) of the peritoneal dialysis system outputs an alert (e.g., alert(s) 1355, alert(s) 1440) identifying the adjustment, for instance to instruct the patient or another user to perform the adjustment, or the inform the patient or other user that the adjustment is being automatically performed by the peritoneal dialysis system, or to ask permission from the patient or other user to perform the adjustment. In some examples, the processor (e.g., controller 160) of the peritoneal dialysis system automatically confirms that the adjustment has been made, and further trains (e.g., update 1455) the trained machine learning model based on further context data (e.g., feedback 1450) to update the trained machine learning model to improve an accuracy of the trained machine learning model for further adjustment identification. In some examples, the further context data indicates an effect of the adjustment on the function.

In some examples, the processor (e.g., controller 160) of the peritoneal dialysis system processes context data (e.g., context data 1410) using a trained machine learning model (e.g., ML model(s) 1425) to identify a sign of an infection (e.g., rise in temperature, pH, conductivity, and/or concentration of substances associated with infection). In some examples, the processor (e.g., controller 160) of the peritoneal dialysis system outputs an alert (e.g., alert(s) 1355, alert(s) 1440) indicative of the infection. In some examples, the processor (e.g., controller 160) of the peritoneal dialysis system automatically confirms that the adjustment has been made, and further trains (e.g., update 1455) the trained machine learning model based on further context data (e.g., feedback 1450) to update the trained machine learning model to improve an accuracy of the trained machine learning model for further infection sign identification. In some examples, the further context data is associated with at least one of confirmation of the infection or treatment of the infection.

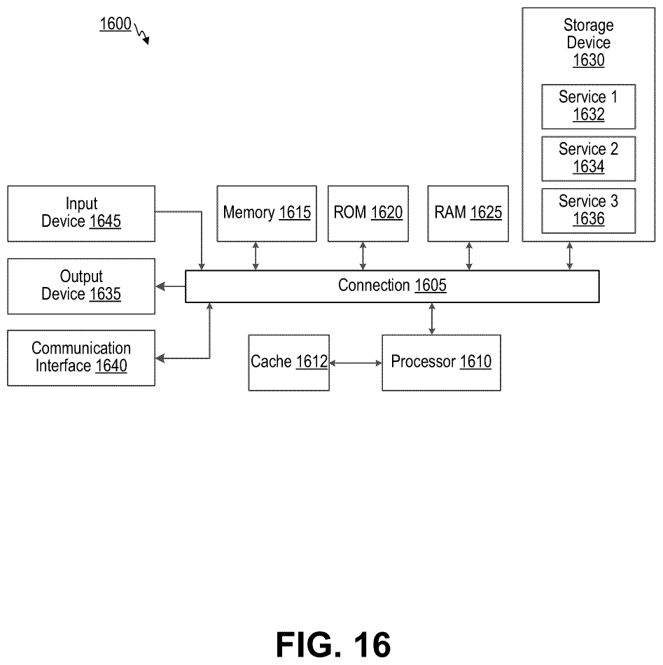

FIG. 16 is a block diagram illustrating an example of a computing system 1600 that can implement the various techniques described herein. In some examples, the computing device can include a mobile device, a wearable device, an extended reality device (e.g., a virtual reality (VR) device, an augmented reality (AR) device, or a mixed reality (MR) device), a personal computer, a laptop computer, a video server, a vehicle (or computing device of a vehicle), or other device. For example, the computing system 1600 may include, implement, or be included in any or all of the controller 160 of any of FIGS. 1-5, a controller (e.g., the controller 160) that controls the filter subsystem 605, a controller (e.g., controller 160) that controls the waste evaporator subsystem 805, the user device 1350, the machine learning engine 1420, the ML model(s) 1425, the feedback engine(s) 1445, the peritoneal dialysis system that performs the process 1500, or a combination thereof. Additionally or alternatively, the computing system 1600 may be configured to perform process 1500 of FIG. 15, and/or any other process described herein.

In particular, FIG. 16 illustrates an example of computing system 1600, which can be for example any computing device making up internal computing system, a remote computing system, a camera, or any component thereof in which the components of the system are in communication with each other using connection 1605. Connection 1605 can be a physical connection using a bus, or a direct connection into processor 1610, such as in a chipset architecture. Connection 1605 can also be a virtual connection, networked connection, or logical connection.

In some aspects, computing system 1600 is a distributed system in which the functions described in this disclosure can be distributed within a datacenter, multiple data centers, a peer network, etc. In some aspects, one or more of the described system components represents many such components each performing some or all of the function for which the component is described. In some aspects, the components can be physical or virtual devices.

Example system 1600 includes at least one processing unit (CPU or processor) 1610 and connection 1605 that communicatively couples various system components including system memory (e.g., memory unit 1615), such as read-only memory (ROM) 1620 and random access memory (RAM) 1625 to processor 1610. Computing system 1600 can include a cache 1612 of high-speed memory connected directly with, in close proximity to, or integrated as part of processor 1610.

Processor 1610 can include any general purpose processor and a hardware service or software service, such as services 1632, 1634, and 1636 stored in storage device 1630, configured to control processor 1610 as well as a special-purpose processor where software instructions are incorporated into the actual processor design. Processor 1610 may essentially be a completely self-contained computing system, containing multiple cores or processors, a bus, memory controller, cache, etc. A multi-core processor may be symmetric or asymmetric.

To enable user interaction, computing system 1600 includes an input device 1645, which can represent any number of input mechanisms, such as a microphone for speech, a touch-sensitive screen for gesture or graphical input, keyboard, mouse, motion input, speech, etc. Computing system 1600 can also include output device 1635, which can be one or more of a number of output mechanisms. In some instances, multimodal systems can enable a user to provide multiple types of input/output to communicate with computing system 1600.

Computing system 1600 can include communications interface 1640, which can generally govern and manage the user input and system output. The communication interface may perform or facilitate receipt and/or transmission wired or wireless communications using wired and/or wireless transceivers, including those making use of an audio jack/plug, a microphone jack/plug, a universal serial bus (USB) port/plug, an Apple™ Lightning™ port/plug, an Ethernet port/plug, a fiber optic port/plug, a proprietary wired port/plug, 3G, 4G, 5G and/or other cellular data network wireless signal transfer, a Bluetooth™ wireless signal transfer, a Bluetooth™ low energy (BLE) wireless signal transfer, an IBEACON™ wireless signal transfer, a radio-frequency identification (RFID) wireless signal transfer, near-field communications (NFC) wireless signal transfer, dedicated short range communication (DSRC) wireless signal transfer, 902.11 Wi-Fi wireless signal transfer, wireless local area network (WLAN) signal transfer, Visible Light Communication (VLC), Worldwide Interoperability for Microwave Access (WiMAX), Infrared (IR) communication wireless signal transfer, Public Switched Telephone Network (PSTN) signal transfer, Integrated Services Digital Network (ISDN) signal transfer, ad-hoc network signal transfer, radio wave signal transfer, microwave signal transfer, infrared signal transfer, visible light signal transfer, ultraviolet light signal transfer, wireless signal transfer along the electromagnetic spectrum, or some combination thereof.

The communications interface 1640 may also include one or more range sensors (e.g., LIDAR sensors, laser range finders, RF radars, ultrasonic sensors, and infrared (IR) sensors) configured to collect data and provide measurements to processor 1610, whereby processor 1610 can be configured to perform determinations and calculations needed to obtain various measurements for the one or more range sensors. In some examples, the measurements can include time of flight, wavelengths, azimuth angle, elevation angle, range, linear velocity and/or angular velocity, or any combination thereof. The communications interface 1640 may also include one or more Global Navigation Satellite System (GNSS) receivers or transceivers that are used to determine a location of the computing system 1600 based on receipt of one or more signals from one or more satellites associated with one or more GNSS systems. GNSS systems include, but are not limited to, the US-based GPS, the Russia-based Global Navigation Satellite System (GLONASS), the China-based BeiDou Navigation Satellite System (BDS), and the Europe-based Galileo GNSS. There is no restriction on operating on any particular hardware arrangement, and therefore the basic features here may easily be substituted for improved hardware or firmware arrangements as they are developed.

Storage device 1630 can be a non-volatile and/or non-transitory and/or computer-readable memory device and can be a hard disk or other types of computer readable media which can store data that are accessible by a computer, such as magnetic cassettes, flash memory cards, solid state memory devices, digital versatile disks, cartridges, a floppy disk, a flexible disk, a hard disk, magnetic tape, a magnetic strip/stripe, any other magnetic storage medium, flash memory, memristor memory, any other solid-state memory, a compact disc read only memory (CD-ROM) optical disc, a rewritable compact disc (CD) optical disc, digital video disk (DVD) optical disc, a blu-ray disc (BDD) optical disc, a holographic optical disk, another optical medium, a secure digital (SD) card, a micro secure digital (microSD) card, a Memory Stick® card, a smartcard chip, a EMV chip, a subscriber identity module (SIM) card, a mini/micro/nano/pico SIM card, another integrated circuit (IC) chip/card, random access memory (RAM), static RAM (SRAM), dynamic RAM (DRAM), read-only memory (ROM), programmable read-only memory (PROM), erasable programmable read-only memory (EPROM), electrically crasable programmable read-only memory (EEPROM), flash EPROM (FLASHEPROM), cache memory (e.g., Level 1 (L1) cache, Level 2 (L2) cache, Level 3 (L3) cache, Level 4 (L4) cache, Level 5 (L5) cache, or other (L #) cache), resistive random-access memory (RRAM/ReRAM), phase change memory (PCM), spin transfer torque RAM (STT-RAM), another memory chip or cartridge, and/or a combination thereof.

The storage device 1630 can include software services, servers, services, etc., that when the code that defines such software is executed by the processor 1610, it causes the system to perform a function. In some aspects, a hardware service that performs a particular function can include the software component stored in a computer-readable medium in connection with the necessary hardware components, such as processor 1610, connection 1605, output device 1635, etc., to carry out the function. The term “computer-readable medium” includes, but is not limited to, portable or non-portable storage devices, optical storage devices, and various other mediums capable of storing, containing, or carrying instruction(s) and/or data. A computer-readable medium may include a non-transitory medium in which data can be stored and that does not include carrier waves and/or transitory electronic signals propagating wirelessly or over wired connections. Examples of a non-transitory medium may include, but are not limited to, a magnetic disk or tape, optical storage media such as compact disk (CD) or digital versatile disk (DVD), flash memory, memory or memory devices. A computer-readable medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like.

Specific details are provided in the description above to provide a thorough understanding of the aspects and examples provided herein, but those skilled in the art will recognize that the application is not limited thereto. Thus, while illustrative aspects of the application have been described in detail herein, it is to be understood that the inventive concepts may be otherwise variously embodied and employed, and that the appended claims are intended to be construed to include such variations, except as limited by the prior art. Various features and aspects of the above-described application may be used individually or jointly. Further, aspects can be utilized in any number of environments and applications beyond those described herein without departing from the broader scope of the specification. The specification and drawings are, accordingly, to be regarded as illustrative rather than restrictive. For the purposes of illustration, methods were described in a particular order. It should be appreciated that in alternate aspects, the methods may be performed in a different order than that described.

For clarity of explanation, in some instances the present technology may be presented as including individual functional blocks comprising devices, device components, steps or routines in a method embodied in software, or combinations of hardware and software. Additional components may be used other than those shown in the figures and/or described herein. For example, circuits, systems, networks, processes, and other components may be shown as components in block diagram form in order not to obscure the aspects in unnecessary detail. In other instances, well-known circuits, processes, algorithms, structures, and techniques may be shown without unnecessary detail in order to avoid obscuring the aspects.

Further, those of skill in the art will appreciate that the various illustrative logical blocks, modules, circuits, and algorithm steps described in connection with the aspects disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

Individual aspects may be described above as a process or method which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination can correspond to a return of the function to the calling function or the main function.

Processes and methods according to the above-described examples can be implemented using computer-executable instructions that are stored or otherwise available from computer-readable media. Such instructions can include, for example, instructions and data which cause or otherwise configure a general purpose computer, special purpose computer, or a processing device to perform a certain function or group of functions. Portions of computer resources used can be accessible over a network. The computer executable instructions may be, for example, binaries, intermediate format instructions such as assembly language, firmware, source code. Examples of computer-readable media that may be used to store instructions, information used, and/or information created during methods according to described examples include magnetic or optical disks, flash memory, USB devices provided with non-volatile memory, networked storage devices, and so on.

In some aspects the computer-readable storage devices, mediums, and memories can include a cable or wireless signal containing a bitstream and the like. However, when mentioned, non-transitory computer-readable storage media expressly exclude media such as energy, carrier signals, electromagnetic waves, and signals per se.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof, in some cases depending in part on the particular application, in part on the desired design, in part on the corresponding technology, etc.

The various illustrative logical blocks, modules, and circuits described in connection with the aspects disclosed herein may be implemented or performed using hardware, software, firmware, middleware, microcode, hardware description languages, or any combination thereof, and can take any of a variety of form factors. When implemented in software, firmware, middleware, or microcode, the program code or code segments to perform the necessary tasks (e.g., a computer-program product) may be stored in a computer-readable or machine-readable medium. A processor(s) may perform the necessary tasks. Examples of form factors include laptops, smart phones, mobile phones, tablet devices or other small form factor personal computers, personal digital assistants, rackmount devices, standalone devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are example means for providing the functions described in the disclosure.

The techniques described herein may also be implemented in electronic hardware, computer software, firmware, or any combination thereof. Such techniques may be implemented in any of a variety of devices such as general purposes computers, wireless communication device handsets, or integrated circuit devices having multiple uses including application in wireless communication device handsets and other devices. Any features described as modules or components may be implemented together in an integrated logic device or separately as discrete but interoperable logic devices. If implemented in software, the techniques may be realized at least in part by a computer-readable data storage medium comprising program code including instructions that, when executed, performs one or more of the methods, algorithms, and/or operations described above. The computer-readable data storage medium may form part of a computer program product, which may include packaging materials. The computer-readable medium may comprise memory or data storage media, such as random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The techniques additionally, or alternatively, may be realized at least in part by a computer-readable communication medium that carries or communicates program code in the form of instructions or data structures and that can be accessed, read, and/or executed by a computer, such as propagated signals or waves.

The program code may be executed by a processor, which may include one or more processors, such as one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A general-purpose processor may be a microprocessor; but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Accordingly, the term “processor,” as used herein may refer to any of the foregoing structure, any combination of the foregoing structure, or any other structure or apparatus suitable for implementation of the techniques described herein.

One of ordinary skill will appreciate that the less than (“<”) and greater than (“>”) symbols or terminology used herein can be replaced with less than or equal to (“_”) and greater than or equal to (“>”) symbols, respectively, without departing from the scope of this description.

Where components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

The phrase “coupled to” or “communicatively coupled to” refers to any component that is physically connected to another component either directly or indirectly, and/or any component that is in communication with another component (e.g., connected to the other component over a wired or wireless connection, and/or other suitable communication interface) either directly or indirectly.

Claim language or other language reciting “at least one of” a set and/or “one or more” of a set indicates that one member of the set or multiple members of the set (in any combination) satisfy the claim. For example, claim language reciting “at least one of A and B” or “at least one of A or B” means A, B, or A and B. In another example, claim language reciting “at least one of A, B, and C” or “at least one of A, B, or C” means A, B, C, or A and B, or A and C, or B and C, A and B and C, or any duplicate information or data (e.g., A and A, B and B, C and C, A and A and B, and so on), or any other ordering, duplication, or combination of A, B, and C. The language “at least one of” a set and/or “one or more” of a set does not limit the set to the items listed in the set. For example, claim language reciting “at least one of A and B” or “at least one of A or B” may mean A, B, or A and B, and may additionally include items not listed in the set of A and B. The phrases “at least one” and “one or more” are used interchangeably herein.

Claim language or other language reciting “at least one processor configured to,” “at least one processor being configured to,” “one or more processors configured to,” “one or more processors being configured to,” or the like indicates that one processor or multiple processors (in any combination) can perform the associated operation(s). For example, claim language reciting “at least one processor configured to: X, Y, and Z” means a single processor can be used to perform operations X, Y, and Z; or that multiple processors are each tasked with a certain subset of operations X, Y, and Z such that together the multiple processors perform X, Y, and Z; or that a group of multiple processors work together to perform operations X, Y, and Z. In another example, claim language reciting “at least one processor configured to: X, Y, and Z” can mean that any single processor may only perform at least a subset of operations X, Y, and Z.

Where reference is made to one or more elements performing functions (e.g., steps of a method), one element may perform all functions, or more than one element may collectively perform the functions. When more than one element collectively performs the functions, each function need not be performed by each of those elements (e.g., different functions may be performed by different elements) and/or each function need not be performed in whole by only one element (e.g., different elements may perform different sub-functions of a function). Similarly, where reference is made to one or more elements configured to cause another element (e.g., an apparatus) to perform functions, one element may be configured to cause the other element to perform all functions, or more than one element may collectively be configured to cause the other element to perform the functions.

Where reference is made to an entity (e.g., any entity or device described herein) performing functions or being configured to perform functions (e.g., steps of a method), the entity may be configured to cause one or more elements (individually or collectively) to perform the functions. The one or more components of the entity may include at least one memory, at least one processor, at least one communication interface, another component configured to perform one or more (or all) of the functions, and/or any combination thereof. Where reference to the entity performing functions, the entity may be configured to cause one component to perform all functions, or to cause more than one component to collectively perform the functions. When the entity is configured to cause more than one component to collectively perform the functions, each function need not be performed by each of those components (e.g., different functions may be performed by different components) and/or each function need not be performed in whole by only one component (e.g., different components may perform different sub-functions of a function).

The various illustrative logical blocks, modules, engines, circuits, and algorithm steps described in connection with the embodiments disclosed herein may be implemented as electronic hardware, computer software, firmware, or combinations thereof. To clearly illustrate this interchangeability of hardware and software, various illustrative components, blocks, engines, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present application.

The techniques described herein may also be implemented in electronic hardware, computer software, firmware, or any combination thereof. Such techniques may be implemented in any of a variety of devices such as general purposes computers, wireless communication device handsets, or integrated circuit devices having multiple uses including application in wireless communication device handsets and other devices. Any features described as engines, modules, or components may be implemented together in an integrated logic device or separately as discrete but interoperable logic devices. If implemented in software, the techniques may be realized at least in part by a computer-readable data storage medium comprising program code including instructions that, when executed, performs one or more of the methods described above. The computer-readable data storage medium may form part of a computer program product, which may include packaging materials. The computer-readable medium may comprise memory or data storage media, such as random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The techniques additionally, or alternatively, may be realized at least in part by a computer-readable communication medium that carries or communicates program code in the form of instructions or data structures and that can be accessed, read, and/or executed by a computer, such as propagated signals or waves.

The program code may be executed by a processor, which may include one or more processors, such as one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A general purpose processor may be a microprocessor; but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Accordingly, the term “processor,” as used herein may refer to any of the foregoing structure, any combination of the foregoing structure, or any other structure or apparatus suitable for implementation of the techniques described herein. In addition, in some aspects, the functionality described herein may be provided within dedicated software modules or hardware modules configured for encoding and decoding, or incorporated in a combined video encoder-decoder (CODEC).

Illustrative aspects of the disclosure include:

Aspect 1. An apparatus for peritoneal dialysis, the apparatus comprising: a first lumen that receives a fluid from a first portion of a peritoneal cavity of a patient; a filter that filters the fluid to divide the fluid into a filtered fluid and a waste product; a mixer that adds a predetermined amount of at least one electrolyte to the filtered fluid to produce a dialysate; a sensor that measures one or more characteristics of the dialysate; a processor that executes instructions stored in a memory, wherein execution of the instructions by the processor causes the processor to compare the one or more characteristics to one or more dialysate characteristic thresholds to verify that the dialysate satisfies one or more rules; and a second lumen that provides the dialysate to a second portion of the peritoneal cavity of the patient.

Aspect 2. The apparatus of aspect 1, further comprising: a sorter coupled to the first lumen and the second lumen, wherein the sorter includes a plurality of valves, and wherein the execution of the instructions by the processor causes the sorter to set respective states of each of the plurality of valves to route the fluid from the first lumen to a recycler and to route the dialysate from the recycler to the second lumen, wherein the recycler includes the filter, the mixer, and the sensor.

Aspect 3. The apparatus of aspect 1, wherein the first portion of the peritoneal cavity is further along a direction of gravity than the second portion of the peritoneal cavity.

Aspect 4. The apparatus of aspect 1, wherein the second portion of the peritoneal cavity is further along a direction of gravity than the first portion of the peritoneal cavity.

Aspect 5. The apparatus of aspect 1, further comprising: a first lumen sensor coupled to the first lumen; and a second lumen sensor coupled to the second lumen, wherein the execution of the instructions by the processor causes the processor to determine, based on lumen sensor data from the first lumen sensor and the second lumen sensor, that a tip of the first lumen is further along a direction of gravity than a tip of the second lumen.

Aspect 6. The apparatus of aspect 5, wherein the execution of the instructions by the processor causes the processor to configure a sorter, based on the determination that the tip of the first lumen is further along the direction of gravity than the tip of the second lumen, to receive the fluid from the peritoneal cavity through the first lumen and to provide the dialysate to the peritoneal cavity through the second lumen.

Aspect 7. The apparatus of aspect 5, wherein the execution of the instructions by the processor causes the processor to determine that the tip of the first lumen is further along the direction of gravity than the tip of the second lumen based on a first pressure measured by the first lumen sensor being higher than a second pressure measured by the second lumen sensor.

Aspect 8. The apparatus of aspect 5, wherein the execution of the instructions by the processor causes the processor to: determine, based on additional sensor data received from the first lumen sensor and the second lumen sensor, that the tip of the second lumen is further along the direction of gravity than the tip of the first lumen; and reconfigure a sorter, based on the determination that the tip of the second lumen is further along the direction of gravity than the tip of the first lumen, to receive the fluid from the peritoneal cavity through the second lumen and to provide the dialysate to the peritoneal cavity through the first lumen.

Aspect 9. The apparatus of aspect 5, wherein the execution of the instructions by the processor causes the processor to: determine, based on additional sensor data received from the first lumen sensor and the second lumen sensor, that the tip of the first lumen and the tip of the second lumen are within a threshold distance of one another along the direction of gravity, wherein a plurality of lumens includes the first lumen and the second lumen; and reconfigure a sorter, based on the determination that the tip of the first lumen and the tip of the second lumen are within the threshold distance of one another along the direction of gravity, to receive the fluid from the peritoneal cavity through at least one of the plurality of lumens and to provide the dialysate to the peritoneal cavity through at least the one of the plurality of lumens.

Aspect 10. The apparatus of aspect 5, wherein the first lumen sensor is coupled to a base of the first lumen, wherein the second lumen sensor is coupled to a base of the second lumen.

Aspect 11. The apparatus of aspect 5, wherein the first lumen sensor is coupled to the tip of the first lumen, wherein the second lumen sensor is coupled to the tip of the second lumen.

Aspect 12. The apparatus of aspect 7, further comprising: an inductive charger that provides a wireless power signal to the first lumen sensor and the second lumen sensor to power the capture of the lumen sensor data by the first lumen sensor and the second lumen sensor; and a communication interface that receives the lumen sensor data from the first lumen sensor and the second lumen sensor as a wireless signal.

Aspect 13. The apparatus of aspect 1, further comprising: a waste container that receives the waste product from the filter.

Aspect 14. The apparatus of aspect 13, further comprising: an evaporator that evaporates a liquid from the waste product to reduce a volume of the waste product that is stored in the waste container.

Aspect 15. The apparatus of aspect 1, further comprising: a power supply that provides power to at least the processor, the memory, the sensor, the mixer, and at least one pump, wherein the at least one pump drives the fluid from the peritoneal cavity through the first lumen, wherein the at least one pump drives the dialysate to the peritoneal cavity through the second lumen.

Aspect 16. The apparatus of aspect 1, wherein the execution of the instructions by the processor causes the processor to: process context data using a trained machine learning model to identify an adjustment to make to at least one component of the apparatus to improve a function of the apparatus.

Aspect 17. The apparatus of aspect 16, wherein the execution of the instructions by the processor causes the processor to: automatically perform an action to make the adjustment.

Aspect 18. The apparatus of aspect 16, wherein the execution of the instructions by the processor causes the processor to: output an alert identifying the adjustment.

Aspect 19. The apparatus of aspect 16, wherein the execution of the instructions by the processor causes the processor to: confirm that the adjustment has been made; and further train the trained machine learning model based on further context data to update the trained machine learning model to improve an accuracy of the trained machine learning model for further adjustment identification, wherein the further context data indicates an effect of the adjustment on the function.

Aspect 20. The apparatus of aspect 1, wherein the execution of the instructions by the processor causes the processor to: process context data using a trained machine learning model to identify a sign of an infection.

Aspect 21. The apparatus of aspect 20, wherein the execution of the instructions by the processor causes the processor to: output an alert indicative of the infection.

Aspect 22. The apparatus of aspect 20, wherein the execution of the instructions by the processor causes the processor to: further train the trained machine learning model based on further context data to update the trained machine learning model to improve an accuracy of the trained machine learning model for further infection sign identification, wherein the further context data is associated with at least one of confirmation of the infection or treatment of the infection.

Aspect 23. The apparatus of aspect 1, wherein the at least one electrolyte includes sodium.

Aspect 24. The apparatus of aspect 23, wherein the predetermined amount of at least one electrolyte falls within a range of 130 to 150 milliEquivalents/Liter (mEq/L) of sodium.

Aspect 25. The apparatus of aspect 1, wherein the at least one electrolyte includes at least one of sodium, potassium, chloride, bicarbonate, calcium, or magnesium.

Aspect 26. The apparatus of aspect 1, wherein the filter includes a reverse osmosis (RO) filter.

Aspect 27. The apparatus of aspect 1, further comprising: a recirculation loop that passes at least a subset of the fluid through the filter at least twice.

Aspect 28. The apparatus of aspect 1, wherein the execution of the instructions by the processor causes the processor to: determine the predetermined amount based on sensor data from at last one of the sensor or a second sensor.

Aspect 29. The apparatus of aspect 1, wherein, to verify that the dialysate satisfies one or more rules, the execution of the instructions by the processor causes the processor to: determine that the one or more characteristics of the dialysate are greater than or equal to the one or more dialysate characteristic thresholds, wherein the one or more characteristics and the one or more dialysate characteristic thresholds are associated with at least one of conductivity, potential of hydrogen (pH), temperature, pressure, concentration of the at least one electrolyte in the dialysate, or concentration of at least one other substance in the dialysate.

Aspect 30. The apparatus of aspect 1, wherein, to verify that the dialysate satisfies one or more rules, the execution of the instructions by the processor causes the processor to: determine that the one or more characteristics of the dialysate are less than or equal to the one or more dialysate characteristic thresholds, wherein the one or more characteristics and the one or more dialysate characteristic thresholds are associated with at least one of conductivity, potential of hydrogen (pH), temperature, pressure, concentration of the at least one electrolyte in the dialysate, or concentration of at least one other substance in the dialysate.

Aspect 31. A method of peritoneal dialysis, the method comprising: receiving a fluid from a first portion of a peritoneal cavity of a patient through a first lumen; filtering the fluid using a filter to divide the fluid into a filtered fluid and a waste product; adding a predetermined amount of at least one electrolyte to the filtered fluid to produce a dialysate; measuring one or more characteristics of the dialysate using a sensor; comparing the one or more characteristics to one or more dialysate characteristic thresholds to verify that the dialysate satisfies one or more rules; and providing the dialysate to a second portion of the peritoneal cavity of the patient through a second lumen.

Aspect 32. The method of aspect 31, wherein a sorter is coupled to the first lumen and the second lumen, wherein the sorter includes a plurality of valves, further comprising: setting respective states of each of the plurality of valves to route the fluid from the first lumen to a recycler and to route the dialysate from the recycler to the second lumen, wherein the recycler includes the filter, the mixer, and the sensor.

Aspect 33. The method of aspect 31, wherein the first portion of the peritoneal cavity is further along a direction of gravity than the second portion of the peritoneal cavity.

Aspect 34. The method of aspect 31, wherein the second portion of the peritoneal cavity is further along a direction of gravity than the first portion of the peritoneal cavity.

Aspect 35. The method of aspect 31, further comprising: receiving lumen sensor data from a first lumen sensor and a second lumen sensor, wherein the first lumen sensor is coupled to the first lumen, and wherein the second lumen sensor is coupled to the second lumen; and determining, based on lumen sensor data from the first lumen sensor and the second lumen sensor, that a tip of the first lumen is further along a direction of gravity than a tip of the second lumen.

Aspect 36. The method of aspect 35, further comprising: configuring a sorter, based on the determination that the tip of the first lumen is further along the direction of gravity than the tip of the second lumen, to receive the fluid from the peritoneal cavity through the first lumen and to provide the dialysate to the peritoneal cavity through the second lumen.

Aspect 37. The method of aspect 35, further comprising: determining that the tip of the first lumen is further along the direction of gravity than the tip of the second lumen based on a first pressure measured by the first lumen sensor being higher than a second pressure measured by the second lumen sensor.

Aspect 38. The method of aspect 35, further comprising: determining, based on additional sensor data received from the first lumen sensor and the second lumen sensor, that the tip of the second lumen is further along the direction of gravity than the tip of the first lumen; and reconfiguring a sorter, based on the determination that the tip of the second lumen is further along the direction of gravity than the tip of the first lumen, to receive the fluid from the peritoneal cavity through the second lumen and to provide the dialysate to the peritoneal cavity through the first lumen.

Aspect 39. The method of aspect 35, further comprising: determining, based on additional sensor data received from the first lumen sensor and the second lumen sensor, that the tip of the first lumen and the tip of the second lumen are within a threshold distance of one another along the direction of gravity, wherein a plurality of lumens includes the first lumen and the second lumen; and reconfiguring a sorter, based on the determination that the tip of the first lumen and the tip of the second lumen are within the threshold distance of one another along the direction of gravity, to receive the fluid from the peritoneal cavity through at least one of the plurality of lumens and to provide the dialysate to the peritoneal cavity through at least the one of the plurality of lumens.

Aspect 40. The method of aspect 35, wherein the first lumen sensor is coupled to a base of the first lumen, wherein the second lumen sensor is coupled to a base of the second lumen.

Aspect 41. The method of aspect 35, wherein the first lumen sensor is coupled to the tip of the first lumen, wherein the second lumen sensor is coupled to the tip of the second lumen.

Aspect 42. The method of aspect 37, further comprising: providing, via an inductive charger, a wireless power signal to the first lumen sensor and the second lumen sensor to power the capture of the lumen sensor data by the first lumen sensor and the second lumen sensor; and receiving, via a communication interface, the lumen sensor data from the first lumen sensor and the second lumen sensor as a wireless signal.

Aspect 43. The method of aspect 31, further comprising: receiving, into a waste container, the waste product from the filter.

Aspect 44. The method of aspect 43, further comprising: evaporating, using an evaporator, a liquid from the waste product to reduce a volume of the waste product that is stored in the waste container.

Aspect 45. The method of aspect 31, further comprising: providing, via a power supply, power to at least the processor, the memory, the sensor, the mixer, and at least one pump, wherein the at least one pump drives the fluid from the peritoneal cavity through the first lumen, wherein the at least one pump drives the dialysate to the peritoneal cavity through the second lumen.

Aspect 46. The method of aspect 31, further comprising: processing context data using a trained machine learning model to identify an adjustment to make to at least one component of the method to improve a function of the method.

Aspect 47. The method of aspect 46, further comprising: automatically performing an action to make the adjustment.

Aspect 48. The method of aspect 46, further comprising: outputting an alert identifying the adjustment.

Aspect 49. The method of aspect 46, further comprising: confirming that the adjustment has been made; and further training the trained machine learning model based on further context data to update the trained machine learning model to improve an accuracy of the trained machine learning model for further adjustment identification, wherein the further context data indicates an effect of the adjustment on the function.

Aspect 50. The method of aspect 31, further comprising: processing context data using a trained machine learning model to identify a sign of an infection.

Aspect 51. The method of aspect 50, further comprising: outputting an alert indicative of the infection.

Aspect 52. The method of aspect 50, further comprising: further training the trained machine learning model based on further context data to update the trained machine learning model to improve an accuracy of the trained machine learning model for further infection sign identification, wherein the further context data is associated with at least one of confirmation of the infection or treatment of the infection.

Aspect 53. The method of aspect 31, wherein the at least one electrolyte includes sodium.

Aspect 54. The method of aspect 53, wherein the predetermined amount of at least one electrolyte falls within a range of 130 to 150 milliEquivalents/Liter (mEq/L) of sodium.

Aspect 55. The method of aspect 31, wherein the at least one electrolyte includes at least one of sodium, potassium, chloride, bicarbonate, calcium, or magnesium.

Aspect 56. The method of aspect 31, wherein the filter includes a reverse osmosis (RO) filter.

Aspect 57. The method of aspect 31, further comprising: conveying at least a subset of the fluid through the filter at least twice using a recirculation loop.

Aspect 58. The method of aspect 31, further comprising: determine the predetermined amount based on sensor data from at last one of the sensor or a second sensor.

Aspect 59. The method of aspect 31, wherein verifying that the dialysate satisfies the one or more rules includes: determining that the one or more characteristics of the dialysate are greater than or equal to the one or more dialysate characteristic thresholds, wherein the one or more characteristics and the one or more dialysate characteristic thresholds are associated with at least one of conductivity, potential of hydrogen (pH), temperature, pressure, concentration of the at least one electrolyte in the dialysate, or concentration of at least one other substance in the dialysate.

Aspect 60. The method of aspect 31, wherein verifying that the dialysate satisfies the one or more rules includes: determining that the one or more characteristics of the dialysate are less than or equal to the one or more dialysate characteristic thresholds, wherein the one or more characteristics and the one or more dialysate characteristic thresholds are associated with at least one of conductivity, potential of hydrogen (pH), temperature, pressure, concentration of the at least one electrolyte in the dialysate, or concentration of at least one other substance in the dialysate.

Aspect 61. A non-transitory computer-readable medium having stored thereon instructions that, when executed by one or more processors, cause the one or more processors to perform operations according to any of aspects 1 to 60.

Aspect 62. An apparatus comprising one or more means for performing operations according to any of aspects 1 to 60.

What is claimed is:

1. An apparatus for peritoneal dialysis, the apparatus comprising:
a first lumen that receives a fluid from a first portion of a peritoneal cavity of a patient;
a filter that filters the fluid to divide the fluid into a filtered fluid and a waste product;
a mixer that adds a predetermined amount of at least one electrolyte to the filtered fluid to produce a dialysate;
a sensor that measures one or more characteristics of the dialysate;
a processor that executes instructions stored in a memory, wherein execution of the instructions by the processor causes the processor to compare the one or more characteristics to one or more dialysate characteristic thresholds to verify that the dialysate satisfies one or more rules; and
a second lumen that provides the dialysate to a second portion of the peritoneal cavity of the patient.

2. The apparatus of claim 1, further comprising:
a sorter coupled to the first lumen and the second lumen, wherein the sorter includes a plurality of valves, and wherein the execution of the instructions by the processor causes the sorter to set respective states of each of the plurality of valves to route the fluid from the first lumen to a recycler and to route the dialysate from the recycler to the second lumen, wherein the recycler includes the filter, the mixer, and the sensor.

3. The apparatus of claim 1, wherein the first portion of the peritoneal cavity is further along a direction of gravity than the second portion of the peritoneal cavity.

4. The apparatus of claim 1, wherein the second portion of the peritoneal cavity is further along a direction of gravity than the first portion of the peritoneal cavity.

5. The apparatus of claim 1, further comprising:
a first lumen sensor coupled to the first lumen; and
a second lumen sensor coupled to the second lumen, wherein the execution of the instructions by the processor causes the processor to determine, based on lumen sensor data from the first lumen sensor and the second lumen sensor, that a tip of the first lumen is further along a direction of gravity than a tip of the second lumen.

6. The apparatus of claim 5, wherein the execution of the instructions by the processor causes the processor to configure a sorter, based on the determination that the tip of the first lumen is further along the direction of gravity than the tip of the second lumen, to receive the fluid from the peritoneal cavity through the first lumen and to provide the dialysate to the peritoneal cavity through the second lumen.

7. The apparatus of claim 5, wherein the execution of the instructions by the processor causes the processor to determine that the tip of the first lumen is further along the direction of gravity than the tip of the second lumen based on a first pressure measured by the first lumen sensor being higher than a second pressure measured by the second lumen sensor.

8. The apparatus of claim 5, wherein the execution of the instructions by the processor causes the processor to:
determine, based on additional sensor data received from the first lumen sensor and the second lumen sensor, that the tip of the second lumen is further along the direction of gravity than the tip of the first lumen; and
reconfigure a sorter, based on the determination that the tip of the second lumen is further along the direction of gravity than the tip of the first lumen, to receive the fluid from the peritoneal cavity through the second lumen and to provide the dialysate to the peritoneal cavity through the first lumen.

9. The apparatus of claim 5, wherein the execution of the instructions by the processor causes the processor to:
determine, based on additional sensor data received from the first lumen sensor and the second lumen sensor, that the tip of the first lumen and the tip of the second lumen are within a threshold distance of one another along the direction of gravity, wherein a plurality of lumens includes the first lumen and the second lumen; and
reconfigure a sorter, based on the determination that the tip of the first lumen and the tip of the second lumen are within the threshold distance of one another along the direction of gravity, to receive the fluid from the peritoneal cavity through at least one of the plurality of lumens and to provide the dialysate to the peritoneal cavity through at least the one of the plurality of lumens.

10. The apparatus of claim 5, wherein the first lumen sensor is coupled to a base of the first lumen, wherein the second lumen sensor is coupled to a base of the second lumen.

11. The apparatus of claim 5, wherein the first lumen sensor is coupled to the tip of the first lumen, wherein the second lumen sensor is coupled to the tip of the second lumen.

12. The apparatus of claim 7, further comprising:
an inductive charger that provides a wireless power signal to the first lumen sensor and the second lumen sensor to power the capture of the lumen sensor data by the first lumen sensor and the second lumen sensor; and
a communication interface that receives the lumen sensor data from the first lumen sensor and the second lumen sensor as a wireless signal.

13. The apparatus of claim 1, further comprising:
a waste container that receives the waste product from the filter.

14. The apparatus of claim 13, further comprising:
an evaporator that evaporates a liquid from the waste product to reduce a volume of the waste product that is stored in the waste container.

15. The apparatus of claim 1, further comprising:
a power supply that provides power to at least the processor, the memory, the sensor, the mixer, and at least one pump, wherein the at least one pump drives the fluid from the peritoneal cavity through the first lumen, wherein the at least one pump drives the dialysate to the peritoneal cavity through the second lumen.

16. The apparatus of claim 1, wherein the execution of the instructions by the processor causes the processor to:
process context data using a trained machine learning model to identify an adjustment to make to at least one component of the apparatus to improve a function of the apparatus.

17. The apparatus of claim 16, wherein the execution of the instructions by the processor causes the processor to:
automatically perform an action to make the adjustment.

18. The apparatus of claim 16, wherein the execution of the instructions by the processor causes the processor to:
output an alert identifying the adjustment.

19. The apparatus of claim 16, wherein the execution of the instructions by the processor causes the processor to:
confirm that the adjustment has been made; and
further train the trained machine learning model based on further context data to update the trained machine learning model to improve an accuracy of the trained machine learning model for further adjustment identification, wherein the further context data indicates an effect of the adjustment on the function.

20. The apparatus of claim 1, wherein the execution of the instructions by the processor causes the processor to:
process context data using a trained machine learning model to identify a sign of an infection.

21. The apparatus of claim 20, wherein the execution of the instructions by the processor causes the processor to:
output an alert indicative of the infection.

22. The apparatus of claim 20, wherein the execution of the instructions by the processor causes the processor to:
further train the trained machine learning model based on further context data to update the trained machine learning model to improve an accuracy of the trained machine learning model for further infection sign identification, wherein the further context data is associated with at least one of confirmation of the infection or treatment of the infection.

23. The apparatus of claim 1, wherein the at least one electrolyte includes sodium.

24. The apparatus of claim 23, wherein the predetermined amount of at least one electrolyte falls within a range of 130 to 150 milliEquivalents/Liter (mEq/L) of sodium.

25. The apparatus of claim 1, wherein the at least one electrolyte includes at least one of sodium, potassium, chloride, bicarbonate, calcium, or magnesium.

26. The apparatus of claim 1, wherein the filter includes a reverse osmosis (RO) filter.

27. The apparatus of claim 1, further comprising:
a recirculation loop that passes at least a subset of the fluid through the filter at least twice.

28. The apparatus of claim 1, wherein the execution of the instructions by the processor causes the processor to:
determine the predetermined amount based on sensor data from at last one of the sensor or a second sensor.

29. The apparatus of claim 1, wherein, to verify that the dialysate satisfies one or more rules, the execution of the instructions by the processor causes the processor to:
determine that the one or more characteristics of the dialysate are greater than or equal to the one or more dialysate characteristic thresholds, wherein the one or more characteristics and the one or more dialysate characteristic thresholds are associated with at least one of conductivity, potential of hydrogen (pH), temperature, pressure, concentration of the at least one electrolyte in the dialysate, or concentration of at least one other substance in the dialysate.

30. The apparatus of claim 1, wherein, to verify that the dialysate satisfies one or more rules, the execution of the instructions by the processor causes the processor to:
determine that the one or more characteristics of the dialysate are less than or equal to the one or more dialysate characteristic thresholds, wherein the one or more characteristics and the one or more dialysate characteristic thresholds are associated with at least one of conductivity, potential of hydrogen (pH), temperature, pressure, concentration of the at least one electrolyte in the dialysate, or concentration of at least one other substance in the dialysate.

31. A method of peritoneal dialysis, the method comprising:
receiving a fluid from a first portion of a peritoneal cavity of a patient through a first lumen;
filtering the fluid using a filter to divide the fluid into a filtered fluid and a waste product;
adding a predetermined amount of at least one electrolyte to the filtered fluid to produce a dialysate;
measuring one or more characteristics of the dialysate using a sensor;
comparing the one or more characteristics to one or more dialysate characteristic thresholds to verify that the dialysate satisfies one or more rules; and
providing the dialysate to a second portion of the peritoneal cavity of the patient through a second lumen.
